(19) United States
(12) Patent Application Publication
(10) Pub. No.: US 2008/0176826 A1
(43) Pub. Date: Jul. 24, 2008

(54) Pyridine Compounds For Combating Arthropod Pests and Nematodes

(76) Inventors: Michael Puhl, Lampertheim (DE); Michael Rack, Eppelheim (DE); Thomas Schmidt, Neustadt (DE); Michael Hofmann, Ludwigshafen (DE); Ernst Baumann, Dudenhofen (DE); Markus Kordes, Frankenthal (DE); Wolfgang Von Deyn, Neustadt (DE); David G. Kuhn, Apex, NC (US); Deborah L. Culbertson, Fuquay Varina, NC (US); Douglas D. Anspaugh, Apex, NC (US); Henry Van Tuyl Cotter, Raleigh, NC (US); Hassan Oloumi-Sadeghi, Raleigh, NC (US); Tony Bucci, Fuquay Varina, NC (US); Henricus Maria Martinus Bastiaans, Bartholomaeus-Arnoldi-Str. (NL)

Correspondence Address: HUTCHISON LAW GROUP PLLC, PO BOX 31686, RALEIGH, NC, 27612, US

(21) Appl. No.: 11/914,996

(22) PCT Filed: May 24, 2006
(86) PCT No.: PCT/EP2006/004992

§ 371 Date: Nov. 20, 2007

Related U.S. Application Data
(60) Provisional application No. 60/684,614, filed on May 25, 2005.

(51) Int. Cl. A01N 43/44; C07D 401/12; A01P 7/00; A01N 43/40

(52) U.S. Cl. 514/210.2; 546/278.4; 514/343

(57) ABSTRACT

The present invention relates to new pyridine compounds which are useful for combating animal pests, in particular insects, arachnids and nematodes. The invention also relates to a method for combating insects, nematodes and arachnids.

The pyridine compounds have the formula I as defined below. Likewise, compounds of the formula II are suitable for combating pests.

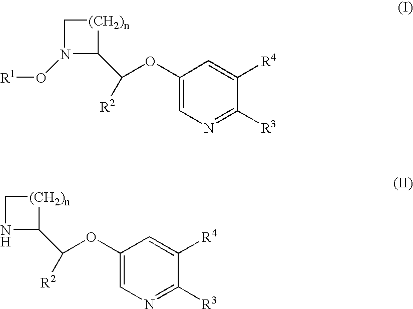

In formulae I and II n is 1 or 2, and R¹, R², R³, R⁴ and R⁵ are as defined in the claims and in the specification.

The present invention relates to new pyridine compounds which are useful for combating animal pests, in particular insects, arachnids and nematodes. The invention also relates to a method for combating arthropod pests, in particular insects, nematodes and arachnids.

In spite of commercial pesticides available today, damage to crops, both growing and harvested, the damage of non-living material, in particular cellulose based materials such as wood or paper, caused by arthropod and nematode pests still occur. Therefore, there is continuing need to provide compounds which are useful for combating such pests.

WO 01/80844, WO 02/87288 and US 2003/0069272 inter alia mention compounds of the general formula A

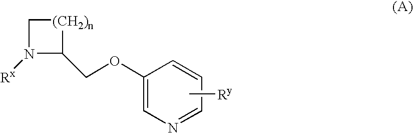

wherein n is 1, 2 or 3, R^{x} is H, C₁-C₆-alkyl, C₂-C₆-alkenyl, C₂-C₆-alkynyl, C₃-C₇-cycloalkyl, C₄-C₇-cycloalkenyl, C₁-C₆-alkoxy or amino and R^{y} is H, C₁-C₆-alkyl, C₂-C₆-alkenyl, C₂-C₆-alkynyl, C₃-C₇-cycloalkyl, C₄-C₇-cycloalkenyl, C₁-C₆-alkoxy, Cl, Br, I, or amino. The compounds are taught to be useful for treating dry eye disease, vaginal dryness or for altering the amount or composition of synovials fluids secreted from joints. However, these documents do not mention how to obtain these compounds.

Similar compounds, wherein R^{x} is H or alkyl are e.g. known from WO 94/08992, WO 96/40682, WO 98/25920 and U.S. Pat. No. 5,629,325. These compounds are mentioned to be useful for preparing pharmaceutical compositions for selectively controlling synaptic transmissions in mammals, or to be useful as analgetics, as well as neuronal cell death preventros and anti-inflammatories.

It is an object of the present invention to provide compounds that have a good activity against harmful arthropod pests, such as insects andarachnids, or nematodes, and which thus are useful for combating said pests.

The inventors of the present application surprisingly found that this object is achieved by compounds of formulae I and II as defined below and by the salts thereof:

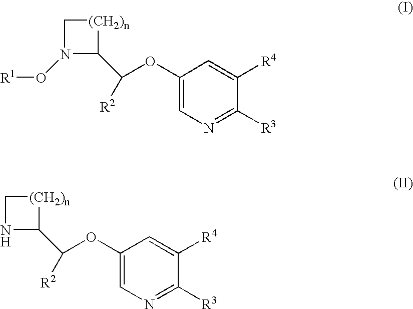

In formulae I and II n is 1 or 2,
R¹ is selected from the group consisting of hydrogen, C₁-C₆-alkyl, C₂-C₆-alkenyl, C₂-C₆-alkynyl, C₃-C₆-cycloalkyl, C₃-C₆-cycloalkyl-C₁-C₄-alkyl, phenyl-C₁-C₄-alkyl, wherein C₁-C₆-alkyl, C₂-C₆-alkenyl, C₂-C₆-alkynyl, C₃-C₆-cycloalkyl, C₃-C₆-cycloalkyl-C₁-C₄-alkyl may be unsubstituted or may be partially or fully halogenated or carry a radical R^{a} selected from OH, CN, C₁-C₄-alkoxy, C₁-C₄-haloalkoxy, —S—C₁-C₄-alkyl, —S(O)—C₁-C₄-alkyl, —S(O)₂—C₁-C₄-alkyl, —S—C₁-C₄-haloalkyl, —S(O)—C₁-C₄-haloalkyl, —S(O)₂—C₁-C₄-haloalkyl, NH₂, NH—C₁-C₄-alkyl, N(C₁-C₄-alkyl)₂, —C(O)—C₁-C₄-alkyl, —C(O)—C₁-C₄-haloalkyl, —C(O)—O—C₁-C₄-alkyl, C(O)NH₂, —C(O)—NH—C₁-C₄-alkyl and —C(O)—N—(C₁-C₄-alkyl)₂ and wherein phenyl in phenyl-C₁-C₄-alkyl may carry 1, 2, 3, 4 or 5 radicals selected from halogen, C₁-C₄-haloalkyl, NO₂ and radicals R^{a},
   or R¹ is phenyl or which may carry 1, 2, 3, 4 or 5 radicals selected from halogen, C₁-C₄-haloalkyl, NO₂ and radicals R^{a},
   or R¹ is a radical of the formula C(O)—R⁵;
R² is hydrogen or C₁-C₄-alkyl;
R³, R⁴ are selected independently of one another from H, halogen, CN, C₁-C₄-alkyl, C₁-C₄-haloalkyl, C₁-C₄-alkoxy, —S—C₁-C₄-alkyl, —S(O)—C₁-C₄-alkyl, S(O)₂—C₁-C₄-alkyl, —S—C₁-C₄-haloalkyl, —S(O)—C₁-C₄-haloalkyl, S(O)₂—C₁-C₄-haloalkyl; and
R⁵ is selected from hydrogen, C₁-C₆-alkyl, C₂-C₆-alkenyl, C₂-C₆-alkynyl, C₁-C₄-haloalkyl, C₂-C₄-haloalkenyl and C₂-C₄-haloalkynyl, wherein C₁-C₆-alkyl, C₂-C₆-alkenyl, C₂-C₆-alkynyl and C₁-C₆-alkoxy, may be unsubstituted or may carry a radical R^{b} which is selected from selected from OH, CN, C₁-C₄-alkoxy, C₁-C₄-haloalkoxy, —S—C₁-C₄-alkyl, —S(O)—C₁-C₄-alkyl, —S(O)₂—C₁-C₄-alkyl, —S—C₁-C₄-haloalkyl, —S(O)—C₁-C₄-haloalkyl, —S(O)₂—C₁-C₄-haloalkyl, NH₂, NH—C₁-C₄-alkyl, N(C₁-C₄-alkyl)₂, —C(O)—C₁-C₄-alkyl, —C(O)—C₁-C₄-haloalkyl, —C(O)—O—C₁-C₄-alkyl, C(O)NH₂, —C(O)—NH—C₁-C₄-alkyl and —C(O)—N—(C₁-C₄-alkyl)₂,
   R⁵ may also be O—R⁶, NR⁷R⁸, C₃-C₆-cycloalkyl, aryl, hetaryl, C₃-C₆-cycloalkyl-C₁-C₄-alkyl, aryl-C₁-C₄-alkyl and hetaryl-C₁-C₄-alkyl, wherein cycloalkyl, aryl and hetaryl in the six last mentioned radicals may be unsubstituted or may carry 1, 2, 3, 4 or 5 radicals R^{c}, selected from halogen, OH, CN, NO₂, C₁-C₄-alkyl, C₁-C₄-haloalkyl, O—R⁹, S(O)ₖR¹⁰, NR¹¹—C(O)—R¹², NR¹³—SO₂—R¹⁴, —C(O)—R¹⁵, —C(O)—O—R¹⁶, —NR¹⁷R¹³, —C(O)—NR¹⁹R²⁰ and —S(O)₂—NR²¹R²², it being possible for two radicals R^{c} which are bound to adjacent carbon atoms to form a C₃-C₆ alkylen moiety or a O—(C₁-C₄-alkylene)-O moiety, wherein the hydrogen atoms of alkylene may be replaced by halogen atoms;
   k is 0, 1 or 2;
   R⁶, R⁹R¹⁰, R¹⁴ independently of each other are C₁-C₄-alkyl, C₁-C₄-haloalkyl, C₃-C₄-alkenyl, C₃-C₄-alkynyl, C₃-C₆-cycloalkyl, C₃-C₆-cycloalkyl-C₁-C₄-alkyl, phenyl and phenyl-C₁-C₄-alkyl, wherein phenyl in the last two mentioned radicals may be unsubstituted or may carry 1, 2, 3, 4 or 5 radicals R^{d}, where R^{d} is selected from halogen, OH, CN, NO₂, C₁-C₄-alkyl, C₁-C₄-haloalkyl, C₁-C₄-alkoxy and C₁-C₄-haloalkoxy;
   R⁷, R⁸, R¹¹, R¹³, R¹⁷, R¹⁸, R¹⁹, R²⁰, R²¹, R²² independently of each other are selected from hydrogen and C₁-C₄-alkyl, C₃-C₄-alkenyl and C₃-C₄-alkynyl;
   R¹², R¹⁵, R¹⁶ independently of each other are selected from hydrogen, C₁-C₄-alkyl, C₁-C₄-haloalkyl, C₃-C₄-alkenyl, C₃-C₄-alkynyl, C₃-C₆-cycloalkyl, C₃-C₆-cycloalkyl-C₁-C₄-alkyl, phenyl and phenyl-C₁-C₄-alkyl, wherein phenyl in the last two mentioned radicals may be unsubstituted or may carry 1, 2, 3, 4 or 5 radicals R^{e}, where R^{e} is selected from halogen, OH, CN, NO₂, C₁-C₄-alkyl, C₁-C₄-haloalkyl, C₁-C₄-alkoxy and C₁-C₄-haloalkoxy;

This object is also achieved by the pyridine N-oxides of compounds of formulae I and II as defined herein and by the salts thereof:

Compounds of the formula I as defined herein and their pyridine N-oxides and the salts thereof have not yet been described in the art. Therefore, the present invention relates to pyridine compound of the general formula I as herein defined, to the pyridine N-oxides and to the salts thereof.

Likewise, compounds of the formula IIa (which correspond to compounds of the formula II, wherein n is 1 and R² is C₁-C₄-alkyl), their salts and their pyridine N-oxides have not yet been described in the art.

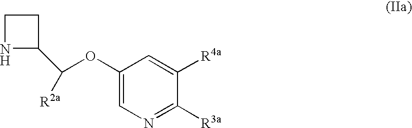

Therefore, the present invention also relates to pyridine compound of the general formula IIa as herein defined, to the pyridine N-oxides of IIa and to the salts thereof.

Due to their excellent activity, the pyridine compounds of the general formulae I and II, their salts and their pyridine N-oxides can be used for controlling arthropod pests and also nematode pests. The term arthropod pest as used herein, include any pests belonging to the tribe of arthropods, in particular pests selected from the orders of insects and arachnids. The compounds of the formulae I and II, their salts and their pyridine N-oxides are in particular useful from combating insects.

Therefore the invention also relates to a method for combating arthropod pests, in particular arthropod pests selected from insects and arachnids. The present invention also relates to a method for combating nematodes. These methods comprise contacting said pests, their habitat, breeding ground, food supply, plant, seed, soil, area, material or environment in which the animal pests are growing or may grow, or the materials, plants, seeds, soils, surfaces or spaces to be protected from an attack of or infestation by said pest, with a pesticidally effective amount of a pyridine compound of the general formulae I or II as defined herein, or with a pyridine N-oxide of I or II or with a salt thereof.

Accordingly, the invention further provides compositions for combating such pests, preferably in the form of directly sprayable solutions, emulsions, pastes, oil dispersions, powders, materials for scattering, dusts or in the form of granules, which comprises a pesticidally effective amount of at least one compound of the general formulae I or II or at least a pyridine N-oxide thereof or at least a salt thereof and at least one carrier which may be liquid and/or solid and which is preferably agriculturally acceptable, and/or at least one surfactant.

The invention provides in particular a method for protecting crops, including seeds, from attack or infestation by arthropod pests, in particular harmful insects and/or arachnids, said method comprises contacting a crop with a pesticidally effective amount of at least one compound of formulae I or II as defined herein or with at least a pyridine N-oxide thereof or with a salt thereof. The invention also provides a method for protecting crops, including seeds, from attack or infestation by nematodes, said method comprises contacting a crop with a pesticidally effective amount of at least one compound of formulae I or II as defined herein or with at least a pyridine N-oxide thereof or with a salt thereof.

The invention also provides a method for protecting non-living materials from attack or infestation by the aforementioned pests, which method comprises contacting the non-living material with a pesticidally effective amount of at least one compound of formulae I or II as defined herein or with at least a pyridine N-oxide thereof or with a salt thereof.

Suitable compounds of the general formulae I and II (and likewise the pyridine N-oxides) encompass all possible stereoisomers (cis/trans isomers, enantiomers) which may occur and mixtures thereof. The present invention provides both the pure enantiomers or diastereomers or mixtures thereof, the pure cis- and trans-isomers and the mixtures thereof.

The invention comprises in particular the enantiomer I-R having R-configuration at the indicated carbon atom and the enantiomer I-S having S-configuration at the indicated carbon atom as well as mixtures thereof.

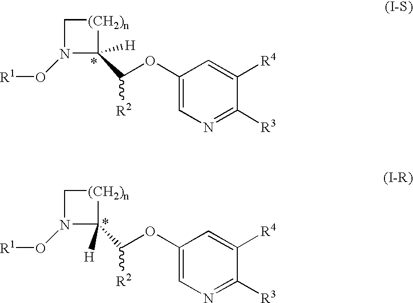

The invention likewise comprises in particular the use of enantiomer II-R having R-configuration at the indicated carbon atom and of the enantiomer II-S having S-configuration at the indicated carbon atom as well as the use of mixtures thereof.

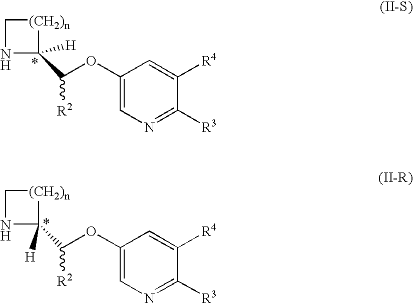

Salts of the compounds of the formulae I and II (and likewise of the pyridine N-oxides of I or II) are preferably agriculturally acceptable salts. They can be formed in a customary method, e.g. by reacting the compound with an acid of the anion in question if the compound of formulae I or II or the pyridine N-oxide thereof has a basic functionality or by reacting an acidic compound of formulae I or II with a suitable base.

Suitable agriculturally useful salts are especially the salts of those cations or the acid addition salts of those acids whose cations and anions, respectively, do not have any adverse effect on the action of the compounds according to the present invention. Suitable cations are in particular the ions of the alkali metals, preferably lithium, sodium and potassium, of the alkaline earth metals, preferably calcium, magnesium and barium, and of the transition metals, preferably manganese, copper, zinc and iron, and also ammonium (NH₄⁺) and substituted ammonium in which one to four of the hydrogen atoms are replaced by C₁-C₄-alkyl, C₁-C₄-hydroxyalkyl, C₁-C₄-alkoxy, C₁-C₄-alkoxy-C₁-C₄-alkyl, hydroxy-C₁-C₄-alkoxy-C₁-C₄-alkyl, phenyl or benzyl. Examples of substituted ammonium ions comprise methylammonium, isopropylammonium, dimethylammonium, diisopropylammonium, trimethylammonium, tetramethylammonium, tetraethylammonium, tetrabutylammonium, 2-hydroxyethylammonium, 2-(2-hydroxyethoxy)ethylammonium, bis(2-hydroxyethyl)ammonium, benzyltrimethylammonium and benzyltriethylammonium, furthermore phosphonium ions, sulfonium ions, preferably tri(C₁-C₄-alkyl)sulfonium, and sulfoxonium ions, preferably tri(C₁-C₄-alkyl)sulfoxonium.

Anions of useful acid addition salts are primarily chloride, bromide, fluoride, hydrogen sulphate, sulphate, dihydrogen phosphate, hydrogen phosphate, phosphate, nitrate, hydrogen carbonate, carbonate, hexafluorosilicate, hexafluorophosphate, benzoate, and the anions of C₁-C₄-alkanoic acids, preferably formate, acetate, propionate and butyrate. They can be formed by reacting the compounds of the formulae Ia and Ib with an acid of the corresponding anion, preferably of hydrochloric acid, hydrobromic acid, sulfuric acid, phosphoric acid or nitric acid.

The organic moieties mentioned in the above definitions of the variables are—like the term halogen—collective terms for individual listings of the individual group members. The prefix Cₙ-Cₘ indicates in each case the possible number of carbon atoms in the group.

“Halogen” will be taken to mean fluoro, chloro, bromo and iodo.

The term “C₁-C₆-alkyl” as used herein refers to a branched or non-branched (linear) saturated hydrocarbon group having 1 to 6 and in particular 1 to 4 carbon atoms, for example methyl, ethyl, propyl, 1-methylethyl, butyl, 1-methylpropyl, 2-methylpropyl, 1,1-dimethylethyl, pentyl, 1-methylbutyl, 2-methylbutyl, 3-methylbutyl, 2,2-dimethylpropyl, 1-ethylpropyl, hexyl, 1,1-dimethylpropyl, 1,2-dimethylpropyl, 1-methylpentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl, 1-ethyl-2-methylpropyl. C₁-C₄-alkyl means for example methyl, ethyl, propyl, 1-methylethyl, butyl, 1-methylpropyl, 2-methylpropyl or 1,1-dimethylethyl.

The term “C₁-C₄-haloalkyl” as used herein refers to a straight-chain or branched alkyl group having 1 to 4 carbon atoms (as mentioned above), where some or all of the hydrogen atoms in these groups may be replaced by halogen atoms as mentioned above, for example chloromethyl, bromomethyl, dichloromethyl, trichloromethyl, fluoromethyl, difluoromethyl, trifluoromethyl, chlorofluoromethyl, dichlorofluoromethyl, chlorodifluoromethyl, 1-chloroethyl, 1-bromoethyl, 1-fluoroethyl, 2-fluoroethyl, 2,2-difluoroethyl, 2,2,2-trifluoroethyl, 2-chloro-2-fluoroethyl, 2-chloro-2,2-difluoroethyl, 2,2-dichloro-2-fluoroethyl, 2,2,2-trichloroethyl, pentafluoroethyl and the like. The term C₁-C₄-haloalkyl in particular comprises C₁-C₂-fluoroalkyl, which is synonym with methyl or ethyl, wherein 1, 2, 3, 4 or 5 hydrogen atoms are substituted by fluorine atoms, such as fluoromethyl, difluoromethyl trifluoromethyl, 1-fluoroethyl, 2-fluoroethyl, 2,2-difluoroethyl, 2,2,2-trifluoroethyl and pentafluoroethyl.

Similarly, “C₁-C₆-alkoxy” refer to straight-chain or branched alkyl groups having 1 to 6 carbon atoms (as mentioned above) bonded through oxygen or sulfur linkages, respectively, at any bond in the alkyl group. Examples include C₁-C₄-alkoxy such as methoxy, ethoxy, propoxy, isopropoxy, butoxy, sec-butoxy, isobutoxy and tert-butoxy.

The term “C₂-C₆-alkenyl” as used herein intends a branched or non-branched unsaturated hydrocarbon group having 2 to 6 carbon atoms and a double bond in any position, such as ethenyl, 1-propenyl, 2-propenyl, 1-methyl-ethenyl, 1-butenyl, 2-butenyl, 3-butenyl, 1-methyl-1-propenyl, 2-methyl-1-propenyl, 1-methyl-2-propenyl, 2-methyl-2-propenyl, 1-pentenyl, 2-pentenyl, 3-pentenyl, 4-pentenyl, 1-methyl-1-butenyl, 2-methyl-1-butenyl, 3-methyl-1-butenyl, 1-methyl-2-butenyl, 2-methyl-2-butenyl, 3-methyl-2-butenyl, 1-methyl-3-butenyl, 2-methyl-3-butenyl, 3-methyl-3-butenyl, 1,1-dimethyl-2-propenyl, 1,2-dimethyl-1-propenyl, 1,2-dimethyl-2-propenyl, 1-ethyl-1-propenyl, 1-ethyl-2-propenyl, 1-hexenyl, 2-hexenyl, 3-hexenyl, 4-hexenyl, 5-hexenyl, 1-methyl-1-pentenyl, 2-methyl-1-pentenyl, 3-methyl-1-pentenyl, 4-methyl-1-pentenyl, 1-methyl-2-pentenyl, 2-methyl-2-pentenyl, 3-methyl-2-pentenyl, 4-methyl-2-pentenyl, 1-methyl-3-pentenyl, 2-methyl-3-pentenyl, 3-methyl-3-pentenyl, 4-methyl-3-pentenyl, 1-methyl-4-pentenyl, 2-methyl-4-pentenyl, 3-methyl-4-pentenyl, 4-methyl-4-pentenyl, 1,1-dimethyl-2-butenyl, 1,1-dimethyl-3-butenyl, 1,2-dimethyl-1-butenyl, 1,2-dimethyl-2-butenyl, 1,2-dimethyl-3-butenyl, 1,3-dimethyl-1-butenyl, 1,3-dimethyl-2-butenyl, 1,3-dimethyl-3-butenyl, 2,2-dimethyl-3-butenyl, 2,3-dimethyl-1-butenyl, 2,3-dimethyl-2-butenyl, 2,3-dimethyl-3-butenyl, 3,3-dimethyl-1-butenyl, 3,3-dimethyl-2-butenyl, 1-ethyl-1-butenyl, 1-ethyl-2-butenyl, 1-ethyl-3-butenyl, 2-ethyl-1-butenyl, 2-ethyl-2-butenyl, 2-ethyl-3-butenyl, 1,1,2-trimethyl-2-propenyl, 1-ethyl-1-methyl-2-propenyl, 1-ethyl-2-methyl-1-propenyl and 1-ethyl-2-methyl-2-propenyl.

The term “C₂-C₆-haloalkenyl” as used herein intends a branched or non-branched unsaturated hydrocarbon group having 2 to 10 carbon atoms and a double bond in any position, where some or all of the hydrogen atoms in these groups may be replaced by halogen atoms as mentioned above.

The term “C₂-C₆-alkynyl” as used herein refers to a branched or non-branched unsaturated hydrocarbon group having 2 to 6 carbon atoms and containing at least one triple bond, such as ethynyl, propynyl, 1-butynyl, 2-butynyl, and the like.

The term “C₂-C₆-haloalkynyl” as used herein refers to a branched or non-branched unsaturated hydrocarbon group having 2 to 6 carbon atoms and containing at least one triple bond, where some or all of the hydrogen atoms in these groups may be replaced by halogen atoms as mentioned above.

The term “C₃-C₆-cycloalkyl” as used herein refers to a monocyclic 3- to 6-membered saturated carbon atom ring, e.g. cyclopropyl, cyclobutyl, cyclopentyl and cyclohexyl.

The term “C₃-C₆-cycloalkyl-C₁-C₄-alkyl” as used herein refers to a monocyclic 3- to 6-membered saturated carbon atom ring which is attached to the remainder of the molecule by an C₁-C₄-alkylene group, e.g. cyclopropylmethyl, 1-cyclopropylethan-1-yl, cyclobutylmethyl, cyclopentylmethyl, cyclohexylmethyl, cycloheptylmethyl, cyclooctylmethyl and cyclodecylmethyl.

The term “C₁-C₄-alkylene” as used herein refers to a saturated bivalent hydrocarbon radical having 1 to 4 carbon atoms such as methylen, ethane-1,2-diyl, ethane-1,1-diyl, propane-1,1-diyl, propane-1,2-diyl, propane-1,3-diyl, propane-2,2-diyl, 2-methylpropan-1,2-diyl, 2-methylpropane-1,3-diyl, butane-1,1-diyl, butane-1,2-diyl, butane-2,2-diyl, butane-2,3-diyl, butane-1,3-diyl and butane-1,4-diyl.

The term “aryl” as used herein refers to an aromatic carbocyclic radical such as phenyl or naphthyl, in particular to phenyl.

The term “aryl-C₁-C₄-alkyl” as used herein refers to an aromatic radical which is attached to the remainder of the molecule by an C₁-C₄-alkylene group, e.g. benzyl, 1-phenylethan-1-yl, 2-phenylethan-1-yl, 2-methyl-2-phenylethan-1-yl and the like.

The term “hetaryl” as used herein refers to an monocyclic aromatic heterocyclic radical and to a fused aromatic heterocyclic radical, in particular to monocyclic 5- or 6-membered aromatic heterocycle, which may comprise a fused benzene ring, wherein the heterocyclic radical, in particular the 5- or 6-membered monocyclic aromatic heterocycle comprises 1, 2, 3 or 4 heteroatoms which are selected, independently of one another, from O, N and S as ring members. Examples for heteroaryl include triazinyl, pyrazinyl, pyrimidinyl, pyridazinyl, pyridyl, thienyl, furyl, pyrrolyl, pyrazolyl, imidazolyl, triazolyl, tetrazolyl, thiazolyl, oxazolyl, 1,2,3-thiadiazolyl, 1,2,4-thiadiazolyl, 1,3,4-thiadiazolyl, 1,2,3-oxadiazolyl, 1,2,4-oxadiazolyl, 1,3,4-oxadiazolyl, isothiazolyl, isoxazolyl, 1,2,3-triazolyl, 1,3,4-triazolyl, indolyl, benzofuranyl, benzoxazolyl, benzpyrazolyl, quinolinyl, isoquinolinyl and the like. Preferred hetaryl comprises pyridinyl, thienyl, furyl, pyrazolyl, oxazolyl, isoxazolyl, thiazolyl, 1,2,3-oxdiazolyl and 1,3,4-oxdiazolyl.

The term “heteroaryl-C₁-C₄-alkyl” as used herein refers to an optionally substituted heteroaryl group which is attached to the remainder of the molecule by an C₁-C₄-alkylene group, examples including triazinylmethyl, pyrazinylmethyl, pyrimidinylmethyl, pyridazinylmethyl, pyridylmethyl, thienylmethyl, furylmethyl, pyrrolylmethyl, pyrazolylmethyl, imidazolyl, triazolylmethyl, tetrazolylmethyl, thiazolylmethyl, oxazolylmethyl, thiadiazolylmethyl, oxadiazolylmethyl, isothiazolylmethyl and isoxazolylmethyl.

In a first preferred embodiment of the invention R¹ in formula I is a radical of the formula C(O)—R⁵ wherein R⁵ is as defined herein. Compounds of this embodiment are referred to as compounds Ia (or I-Ra or I-Sa, respectively, depending of the absolute configuration at the indicated carbon atom *).

R⁵ in formula Ia is preferably a radical selected from C₁-C₆-alkyl, C₂-C₆-alkenyl, C₂-C₆-alkynyl, C₁-C₄-haloalkyl, C₂-C₄-haloalkenyl, C₂-C₄-haloalkynyl, wherein C₁-C₆-alkyl, C₂-C₆-alkenyl, C₂-C₆-alkynyl may be unsubstituted or may carry a radical R^{b} as defined herein.

Likewise preferred are compounds Ia, wherein R⁵ is selected from C₁-C₆-alkoxy, which may be unsubstituted or may carry a radical R^{b} as defined herein.

Likewise preferred are compounds Ia, wherein R⁵ is selected from NH₂, NH—C₁-C₄-alkyl and N(C₁-C₄-alkyl)₂,

Likewise preferred are compounds Ia, wherein R⁵ is selected from C₃-C₆-cycloalkyl and C₃-C₆-cycloalkyl-C₁-C₄-alkyl, wherein cycloalkyl in the two last mentioned radicals may be unsubstituted or may carry 1, 2, 3, 4 or 5 radicals R^{c} as defined herein.

Likewise preferred are compounds Ia, wherein R⁵ is selected from phenyl, naphthyl, phenyl-C₁-C₄-alkyl, hetaryl and hetaryl-C₁-C₄-alkyl, wherein phenyl and hetaryl in the five last mentioned radicals may be unsubstituted or may carry 1, 2, 3, 4 or 5 radicals R^{c} as defined above.

Amongst compounds Ia those are particularly preferred, wherein R⁵ is selected from
H, C₁-C₆-alkyl, C₂-C₆-alkenyl, C₂-C₆-alkynyl, C₁-C₂-haloalkyl,
C₃-C₆-cycloalkyl, naphthyl, phenyl, pyridyl, thienyl, furyl, pyrazolyl, oxazolyl, isoxazolyl, thiazolyl, 1,2,4-oxdiazolyl, 1,3,4-oxdiazolyl, wherein the last 11 radicals may be unsubstituted or may carry 1, 2 or 3 radicals selected from halogen, CN, NO₂, C₁-C₄-alkyl, C₁-C₄-haloalkyl, NH—SO₂—(C₁-C₄-alkyl), C₁-C₄-alkoxy and C₁-C₄-haloalkoxy,
NH₂, C₁-C₄-alkoxy and C₁-C₂-haloalkoxy
   or wherein R⁵ is a radical A-R^{aa} wherein
A is C₁-C₃-alkylen, and
R^{aa} is selected from C₁-C₄-alkoxy, —S—C₁-C₄-alkyl, —S(O)—C₁-C₄-alkyl, S(O)₂—C₁-C₄-alkyl, NH₂, NH—C₁-C₄-alkyl, N(C₁-C₄-alkyl)₂, C₃-C₆-cycloalkyl, phenyl, pyridyl, thienyl, furyl, pyrazolyl, oxazolyl, isoxazolyl, thiazolyl, 1,2,4-oxdiazolyl and 1,3,4-oxdiazolyl, wherein the last 11 radicals may be unsubstituted or may carry 1, 2 or 3 radicals selected from halogen, CN, NO₂, C₁-C₄-alkyl, C₁-C₄-haloalkyl, NH—SO₂—(C₁-C₄-alkyl), C₁-C₄-alkoxy and C₁-C₄-haloalkoxy.

In a second embodiment of the invention R¹ is hydrogen. Compounds of this embodiment are referred to as compounds Ib (or I-Rb or I-Sb, respectively, depending of the absolute configuration at the indicated carbon atom *).

In a third embodiment of the invention R¹ is selected from the group consisting of C₁-C₆-alkyl, C₂-C₆-alkenyl, C₂-C₆-alkynyl, C₃-C₆-cycloalkyl, C₃-C₆-cycloalkyl-C₁-C₄-alkyl, and phenyl-C₁-C₄-alkyl wherein C₁-C₄-alkyl, C₂-C₄-alkenyl, C₂-C₄-alkynyl, C₃-C₆-cycloalkyl and C₃-C₆-cycloalkyl-C₁-C₄-alkyl may be unsubstituted or may be partially or fully halogenated or carry a radical R^{a} as defined herein and wherein phenyl in phenylC₁-C₄-alkyl may be unsubstituted or may carry 1, 2 or 3 radicals selected from halogen, C₁-C₄-haloalkyl, NO₂ and the radicals R^{a} as defined herein. Compounds of this embodiment are referred to as compounds Ic (or I-Rc or I-Sc, respectively, depending of the absolute configuration at the indicated carbon atom *). Amongst compounds Ic, those are preferred, wherein R¹ is selected from C₁-C₆-alkyl, C₂-C₆-alkenyl, C₂-C₆-alkynyl, C₃-C₆-cycloalkyl, or a radical A-R^{ab} wherein A is C₁-C₃-alkylen, in particular methylen, ethane-1,1-diyl or 1,2-ethanediyl, and R^{ab} is selected from C₁-C₄-alkoxy, in particular methoxy or ethoxy, —S—C₁-C₄-alkyl, in particular methylthio or ethylthio, —S(O)—C₁-C₄-alkyl, in particular methylsulfinyl, S(O)₂—C₁-C₄-alkyl, in particular methylsulfonyl, NH₂, NH—C₁-C₄-alkyl, in particular methylamino, ethylamino, N(C₁-C₄-alkyl)₂, in particular dimethylamino, phenyl, which may be unsubstituted or may carry 1, 2 or 3 radicals selected from halogen, NO₂ and the radicals R^{a} as defined herein, or C₃-C₆-cycloalkyl.

In a fourth embodiment of the invention R¹ is phenyl which may be unsubstituted or may carry 1, 2 or 3 radicals selected from halogen, C₁-C₄-haloalkyl, NO₂ and the radicals R^{a} as defined herein. Compounds of this embodiment are referred to as compounds Id (or I-Rd or I-Sd, respectively, depending of the absolute configuration at the indicated carbon atom *).

In a preferred embodiment of the invention R² in formulae Ia, Ib, Ic and Id, and likewise R² in formula II are hydrogen.

In another preferred embodiment of the invention R² in formulae Ia, Ib and Ic, and likewise R² in formula II and R^{2a} in formula IIa are methyl.

In compounds of the formula I (including compounds of the formulae Ia, Ib, Ic), and likewise in compounds of the formulae II and IIa, at least one radical R³ or R⁴ (or R^{3a}, R^{4a}, respectively) is different from hydrogen. In particular R³ and R⁴ are selected, independently of each other, from hydrogen and halogen, more preferably from hydrogen, chlorine, bromine or fluorine.

A very preferred embodiment of the invention relates to compounds of the formula Ia (including formulae I-Ra and I-Sa) as defined herein, wherein n is 1, and wherein R², R³, R⁴ and R⁵ have the meanings as defined herein, in particular the meanings given as preferred. In this embodiment preference is given to compounds, wherein

R² is H or methyl,

R³ is hydrogen or halogen, in particular fluorine, chlorine or bromine,

R⁴ is hydrogen or halogen, in particular fluorine, chlorine or bromine, and

R⁵ is a radical selected from:
C₁-C₆-alkyl, C₂-C₆-alkenyl, C₂-C₆-alkynyl, C₁-C₄-haloalkyl, C₂-C₄-haloalkenyl, C₂-C₄-haloalkynyl, wherein C₁-C₆-alkyl, C₂-C₆-alkenyl, C₂-C₆-alkynyl may be unsubstituted or may carry a radical R^{b} as defined herein,
C₁-C₆-alkoxy, which may be unsubstituted or may carry a radical R^{b} as defined herein,
NH₂, NH—C₁-C₄-alkyl and N(C₁-C₄-alkyl)₂, C₃-C₆-cycloalkyl and C₃-C₆-cycloalkyl-C₁-C₄-alkyl, wherein cycloalkyl in the two last mentioned radicals may be unsubstituted or may carry 1, 2, 3, 4 or 5 radicals R^{c} as defined herein,
phenyl, naphthyl, phenyl-C₁-C₄-alkyl, hetaryl and hetaryl-C₁-C₄-alkyl, wherein phenyl, naphthyl and hetaryl in the five last mentioned radicals may be unsubstituted or may carry 1, 2, 3, 4 or 5 radicals R^{c} as defined herein.
in particular from
H, C₁-C₆-alkyl, C₂-C₆-alkenyl, C₂-C₆-alkynyl, C₁-C₂-haloalkyl, C₃-C₆-cycloalkyl, naphthyl, phenyl, pyridyl, thienyl, furyl, pyrazolyl, oxazolyl, isoxazolyl, thiazolyl, 1,2,4-oxdiazolyl, 1,3,4-oxdiazolyl, wherein the last 11 radicals may be unsubstituted or may carry 1, 2 or 3 radicals selected from halogen, CN, NO₂, C₁-C₄-alkyl, C₁-C₄-haloalkyl, NH—SO₂—(C₁-C₄-alkyl), C₁-C₄-alkoxy and C₁-C₄-haloalkoxy,
NH₂, C₁-C₄-alkoxy, C₁-C₂-haloalkoxy
or wherein R⁵ is a radical A-R^{aa} wherein
A is C₁-C₃-alkylen, and
R^{aa} is selected from C₁-C₄-alkoxy, —S—C₁-C₄-alkyl, —S(O)—C₁-C₄-alkyl, S(O)₂—C₁-C₄-alkyl, NH₂, NH—C₁-C₄-alkyl, N(C₁-C₄-alkyl)₂, C₃-C₆-cycloalkyl, phenyl, pyridyl, thienyl, furyl, pyrazolyl, oxazolyl, isoxazolyl, thiazolyl, 1,2,4-oxdiazolyl and 1,3,4-oxdiazolyl, wherein the last 11 radicals may be unsubstituted or may carry 1, 2 or 3 radicals selected from halogen, CN, NO₂, C₁-C₄-alkyl, C₁-C₄-haloalkyl, NH—SO₂—(C₁-C₄-alkyl), C₁-C₄-alkoxy and C₁-C₄-haloalkoxy.

Another preferred embodiment of the invention relates to compounds of the formula Ia (including formulae I-Ra and I-Sa) as defined herein, wherein n is 2, and wherein R², R³, R⁴ and R⁵ have the meanings as defined herein, in particular the meanings given as preferred.

In this embodiment preference is given to compounds, wherein

R² is H or methyl,

R³ is hydrogen or halogen, in particular fluorine, chlorine or bromine,

R⁴ is hydrogen or halogen, in particular fluorine, chlorine or bromine, and

R⁵ is a radical selected from:
C₁-C₆-alkyl, C₂-C₆-alkenyl, C₂-C₆-alkynyl, C₁-C₄-haloalkyl, C₂-C₄-haloalkenyl, C₂-C₄-haloalkynyl, wherein C₁-C₆-alkyl, C₂-C₆-alkenyl, C₂-C₆-alkynyl may be unsubstituted or may carry a radical R^{b} as defined herein,
C₁-C₆-alkoxy, which may be unsubstituted or may carry a radical R^{b} as defined herein
NH₂, NH—C₁-C₄-alkyl, N(C₁-C₄-alkyl)₂,
C₃-C₆-cycloalkyl, C₃-C₆-cycloalkyl-C₁-C₄-alkyl, phenyl and phenyl-C₁-C₄-alkyl, wherein cycloalkyl and phenyl in the last four mentioned radicals may be unsubstituted or may carry 1, 2, 3, 4 or 5 radicals R^{c} as defined herein.
in particular from
H, C₁-C₆-alkyl, C₂-C₆-alkenyl, C₂-C₆-alkynyl, C₁-C₂-haloalkyl, C₃-C₆-cycloalkyl and phenyl, wherein phenyl and cycloalkyl may be unsubstituted or may carry 1, 2 or 3 radicals selected from halogen, CN, NO₂, C₁-C₄-alkyl, C₁-C₄-haloalkyl, NH—SO₂—(C₁-C₄-alkyl), C₁-C₄-alkoxy and C₁-C₄-haloalkoxy,
NH₂, C₁-C₄-alkoxy, C₁-C₂-haloalkoxy
or wherein R⁵ is a radical A-R^{aa} wherein
A is C₁-C₃-alkylen, and
R^{aa} is selected from C₁-C₄-alkoxy, —S—C₁-C₄-alkyl, —S(O)—C₁-C₄-alkyl, S(O)₂—C₁-C₄-alkyl, NH₂, NH—C₁-C₄-alkyl, N(C₁-C₄-alkyl)₂, C₃-C₆-cycloalkyl or phenyl, wherein phenyl may be unsubstituted or may carry 1, 2 or 3 radicals selected from halogen, CN, NO₂, C₁-C₄-alkyl, C₁-C₄-haloalkyl, NH—SO₂—(C₁-C₄-alkyl), C₁-C₄-alkoxy and C₁-C₄-haloalkoxy.

Apart from that R^{a} and R^{b} are, independently from one another, preferably selected from C₁-C₄-alkoxy, in particular methoxy or ethoxy, —S—C₁-C₄-alkyl, in particular thiomethyl, —S(O)—C₁-C₄-alkyl, in particular methylsulfinyl, S(O)₂—C₁-C₄-alkyl, in particular methylsulfonyl, NH₂, NH—C₁-C₄-alkyl, in particular methylamino or ethylamino, N(C₁-C₄-alkyl)₂, in particular dimethylamino, diethylamino, methylethylamino or methylisopropylamino, phenyl or C₃-C₆-cycloalkyl.

Preferred radicals R^{c} comprise halogen, e.g. F, Cl, Br, CN, NO₂, C₁-C₄-alkyl, in particular methyl, C₁-C₄-haloalkyl, in particular trifluoromethyl, NH—SO₂—(C₁-C₄-alkyl), in particular NH—SO₂—CH₃, C₁-C₄-alkoxy, in particular methoxy, and C₁-C₄-haloalkoxy, in particular trifluoromethoxy, or two radicals R^{c} together may form a C₃-C₆ alkylen moiety, e.g. (CH₂)₃ or (CH₂)₄ or a O—(C₁-C₄-alkylene)-O moiety, wherein the hydrogen atoms of alkylene may be replaced by halogen atoms, e.g. O—CH₂—O, O—CF₂—O or O—C(CH₃)₂—O.

Preferred radicals R^{d}R^{e}, and R^{f}, independently of each other comprise halogen, e.g. F, Cl, Br, CN, NO₂, C₁-C₄-alkyl, in particular methyl, C₁-C₄-haloalkyl, in particular trifluoromethyl, C₁-C₄-alkoxy, in particular methoxy, and C₁-C₄-haloalkoxy, in particular trifluoromethoxy.

Preferred radicals R⁶, R⁹, R¹⁰, R¹⁴ and R¹⁶, independently of each other, are C₁-C₄-alkyl.

Preferred radicals R¹¹, R¹², R¹³, R¹⁵R¹⁷, R¹⁸, R¹⁹, R²⁰, R²¹ and R²², independently of each other, are hydrogen or C₁-C₄-alkyl, or R⁷ and R⁸ (and likewise R¹⁷ and R¹⁸ or R¹⁹ and R²⁰ or R²¹ and R²²) together with the nitrogen atom, to which they are bound form a pyrrolidin, piperidin, morpholin or azepan (=hexahydroazepine) ring.

Examples of preferred compounds Ia, Ia-R and Ia-S (i.e. compounds I, I-R and I-S, wherein R¹ is a radical R₅) are given in the following tables 1 to 32.

### Table 1

Compounds of the formulae Ia, Ia-R and Ia-S, wherein R³ and R⁴ are hydrogen, n is 1 and the combination of R⁵ and R² for one compound corresponds in each case to one row of table A.

### Table 2

Compounds of the formulae Ia, Ia-R and Ia-S, wherein R³ is hydrogen and R⁴ is chlorine, n is 1 and the combination of R⁵ and R² for one compound corresponds in each case to one row of table A.

### Table 3

Compounds of the formulae Ia, Ia-R and Ia-S, wherein R³ is hydrogen and R⁴ is bromine, n is 1 and the combination of R⁵ and R² for one compound corresponds in each case to one row of table A.

### Table 4

Compounds of the formulae Ia, Ia-R and Ia-S, wherein R³ is hydrogen and R⁴ is fluorine, n is 1 and the combination of R⁵ and R² for one compound corresponds in each case to one row of table A.

### Table 5

Compounds of the formulae Ia, Ia-R and Ia-S, wherein R³ is chlorine and R⁴ is hydrogen, n is 1 and the combination of R⁵ and R² for one compound corresponds in each case to one row of table A.

### Table 6

Compounds of the formulae Ia, Ia-R and Ia-S, wherein R³ is chlorine and R⁴ is chlorine, n is 1 and the combination of R⁵ and R² for one compound corresponds in each case to one row of table A.

### Table 7

Compounds of the formulae Ia, Ia-R and Ia-S, wherein R³ is chlorine and R⁴ is bromine, n is 1 and the combination of R⁵ and R² for one compound corresponds in each case to one row of table A.

### Table 8

Compounds of the formulae Ia, Ia-R and Ia-S, wherein R³ is chlorine and R⁴ is fluorine, n is 1 and the combination of R⁵ and R² for one compound corresponds in each case to one row of table A.

### Table 9

Compounds of the formulae Ia, Ia-R and Ia-S, wherein R³ is fluorine and R⁴ is hydrogen, n is 1 and the combination of R⁵ and R² for one compound corresponds in each case to one row of table A.

### Table 10

Compounds of the formulae Ia, Ia-R and Ia-S, wherein R³ is fluorine and R⁴ is chlorine, n is 1 and the combination of R⁵ and R² for one compound corresponds in each case to one row of table A.

### Table 11

Compounds of the formulae Ia, Ia-R and Ia-S, wherein R³ is fluorine and R⁴ is bromine, n is 1 and the combination of R⁵ and R² for one compound corresponds in each case to one row of table A.

### Table 12

Compounds of the formulae Ia, Ia-R and Ia-S, wherein R³ is fluorine and R⁴ is fluorine, n is 1 and the combination of R⁵ and R² for one compound corresponds in each case to one row of table A.

### Table 13

Compounds of the formulae Ia, Ia-R and Ia-S, wherein R³ is bromine and R⁴ is hydrogen, n is 1 and the combination of R⁵ and R² for one compound corresponds in each case to one row of table A.

### Table 14

Compounds of the formulae Ia, Ia-R and Ia-S, wherein R³ is bromine and R⁴ is chlorine, n is 1 and the combination of R⁵ and R² for one compound corresponds in each case to one row of table A.

### Table 15

Compounds of the formulae Ia, Ia-R and Ia-S, wherein R³ is bromine and R⁴ is bromine, n is 1 and the combination of R⁵ and R² for one compound corresponds in each case to one row of table A.

### Table 16

Compounds of the formulae Ia, Ia-R and Ia-S, wherein R³ is bromine and R⁴ is fluorine, n is 1 and the combination of R⁵ and R² for one compound corresponds in each case to one row of table A.

### Table 17

Compounds of the formulae Ia, Ia-R and Ia-S, wherein R³ and R⁴ are hydrogen, n is 2 and the combination of R⁵ and R² for one compound corresponds in each case to one row of table A.

### Table 18

Compounds of the formulae Ia, Ia-R and Ia-S, wherein R³ is hydrogen and R⁴ is chlorine, n is 2 and the combination of R⁵ and R² for one compound corresponds in each case to one row of table A.

### Table 19

Compounds of the formulae Ia, Ia-R and Ia-S, wherein R³ is hydrogen and R⁴ is bromine, n is 2 and the combination of R⁵ and R² for one compound corresponds in each case to one row of table A.

### Table 20

Compounds of the formulae Ia, Ia-R and Ia-S, wherein R³ is hydrogen and R⁴ is fluorine, n is 2 and the combination of R⁵ and R² for one compound corresponds in each case to one row of table A.

### Table 21

Compounds of the formulae Ia, Ia-R and Ia-S, wherein R³ is chlorine and R⁴ is hydrogen, n is 2 and the combination of R⁵ and R² for one compound corresponds in each case to one row of table A.

### Table 22

Compounds of the formulae Ia, Ia-R and Ia-S, wherein R³ is chlorine and R⁴ is chlorine, n is 2 and the combination of R⁵ and R² for one compound corresponds in each case to one row of table A.

### Table 23

Compounds of the formulae Ia, Ia-R and Ia-S, wherein R³ is chlorine and R⁴ is bromine, n is 2 and the combination of R⁵ and R² for one compound corresponds in each case to one row of table A.

### Table 24

Compounds of the formulae Ia, Ia-R and Ia-S, wherein R³ is chlorine and R⁴ is fluorine, n is 2 and the combination of R⁵ and R² for one compound corresponds in each case to one row of table A.

### Table 25

Compounds of the formulae Ia, Ia-R and Ia-S, wherein R³ is fluorine and R⁴ is hydrogen, n is 2 and the combination of R⁵ and R² for one compound corresponds in each case to one row of table A.

### Table 26

Compounds of the formulae Ia, Ia-R and Ia-S, wherein R³ is fluorine and R⁴ is chlorine, n is 2 and the combination of R⁵ and R² for one compound corresponds in each case to one row of table A.

### Table 27

Compounds of the formulae Ia, Ia-R and Ia-S, wherein R³ is fluorine and R⁴ is bromine, n is 2 and the combination of R⁵ and R² for one compound corresponds in each case to one row of table A.

### Table 28

Compounds of the formulae Ia, Ia-R and Ia-S, wherein R³ is fluorine and R⁴ is fluorine, n is 2 and the combination of R⁵ and R² for one compound corresponds in each case to one row of table A.

### Table 29

Compounds of the formulae Ia, Ia-R and Ia-S, wherein R³ is bromine and R⁴ is hydrogen, n is 2 and the combination of R⁵ and R² for one compound corresponds in each case to one row of table A.

### Table 30

Compounds of the formulae Ia, Ia-R and Ia-S, wherein R³ is bromine and R⁴ is chlorine, n is 2 and the combination of R⁵ and R² for one compound corresponds in each case to one row of table A.

### Table 31

Compounds of the formulae Ia, Ia-R and Ia-S, wherein R³ is bromine and R⁴ is bromine, n is 2 and the combination of R⁵ and R² for one compound corresponds in each case to one row of table A.

### Table 32

Compounds of the formulae Ia, Ia-R and Ia-S, wherein R³ is bromine and R⁴ is fluorine, n is 2 and the combination of R⁵ and R² for one compound corresponds in each case to one row of table A.

TABLE A

| | R⁵ | R² |
|---|---|---|
| 1 | H | H |
| 2 | CH₃ | H |
| 3 | CH₂CH₃ | H |
| 4 | (CH₂)₂CH₃ | H |
| 5 | (CH₂)₃CH₃ | H |
| 6 | (CH₂)₄CH₃ | H |
| 7 | (CH₂)₅CH₃ | H |
| 8 | CH(CH₃)₂ | H |
| 9 | CH(CH₃)CH₂CH₃ | H |
| 10 | CH₂CH(CH₃)₂ | H |
| 11 | C(CH₃)₃ | H |
| 12 | C(CH₃)₂CH₂CH₃ | H |
| 13 | CH₂C(CH₃)₃ | H |
| 14 | CH₂-cyclopropyl | H |
| 15 | CH(CH₃)-cyclopropyl | H |
| 16 | CH₂-cyclobutyl | H |
| 17 | CH₂-cylopentyl | H |
| 18 | CH₂-cyclohexyl | H |
| 19 | CH₂OCH₃ | H |
| 20 | CH₂CH₂OCH₃ | H |
| 21 | CF₃ | H |
| 22 | CH₂NH₂ | H |
| 23 | CH₂SCH₃ | H |
| 24 | CH₂SOCH₃ | H |
| 25 | CH₂SO₂CH₃ | H |
| 26 | cyclopropyl | H |
| 27 | cyclobutyl | H |
| 28 | cyclopentyl | H |
| 29 | cyclohexyl | H |
| 30 | CH₂CH═CH₂ | H |
| 31 | CH(CH₃)CH═CH₂ | H |
| 32 | CH₂CHCH═CH₃ | H |
| 33 | CH₂C≡CH | H |
| 34 | C(CH₃)C≡CH | H |
| 35 | C₆H₅ | H |
| 36 | 2-Cl—C₆H₄ | H |
| 37 | 3-Cl—C₆H₄ | H |
| 38 | 4-Cl—C₆H₄ | H |
| 39 | 3,4-Cl₂—C₆H₄ | H |
| 40 | 2,4-Cl₂—C₆H₄ | H |
| 41 | 2,6-Cl₂—C₆H₄ | H |
| 42 | 2,3-Cl₂—C₆H₄ | H |
| 43 | 3,5-Cl₂—C₆H₄ | H |
| 44 | 2-(CH₃)—C₆H₄ | H |
| 45 | 3-(CH₃)—C₆H₄ | H |
| 46 | 4-(CH₃)—C₆H₄ | H |
| 47 | 2-(OCH₃)—C₆H₄ | H |
| 48 | 3-(OCH₃)—C₆H₄ | H |
| 49 | 4-(OCH₃)—C₆H₄ | H |
| 50 | 2,6-Cl₂—C₆H₃ | H |
| 51 | 2,6-(CH₃)₂—C₆H₃ | H |
| 52 | 2,3-(CH₃O)₂—C₆H₃ | H |
| 53 | 2,4-(CH₃O)₂—C₆H₃ | H |
| 54 | 3,4-(CH₃O)₂—C₆H₃ | H |
| 55 | 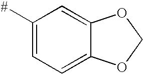 | H |
| 56 | 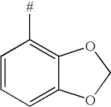 | H |
| 57 | 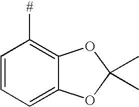 | H |
| 58 | 2-(NO₂)—C₆H₄ | H |
| 59 | 3-(NO₂)—C₆H₄ | H |
| 60 | 4-(NO₂)—C₆H₄ | H |
| 61 | 2-F—C₆H₄ | H |
| 62 | 3-F—C₆H₄ | H |
| 63 | 4-F—C₆H₄ | H |
| 64 | 2-Br—C₆H₄ | H |
| 65 | 3-Br—C₆H₄ | H |
| 66 | 4-Br—C₆H₄ | H |
| 67 | 2-(CF₃)—C₆H₄ | H |
| 68 | 3-(CF₃)—C₆H₄ | H |
| 69 | 4-(CF₃)—C₆H₄ | H |
| 70 | 2-(OCF₃)—C₆H₄ | H |
| 71 | 3-(OCF₃)—C₆H₄ | H |
| 72 | 4-(OCF₃)—C₆H₄ | H |
| 73 | 4-(H₃C—SO₂)—C₆H₄ | H |
| 74 | 2-NC—C₆H₄ | H |
| 75 | 3-NC—C₆H₄ | H |
| 76 | 4-NC—C₆H₄ | H |
| 77 | CH₂—C₆H₅ | H |
| 78 | 1-naphthyl | H |
| 79 | 2-naphthyl | H |
| 80 | 2-pyridyl | H |
| 81 | 3-pyridyl | H |
| 82 | 4-pyridyl | H |
| 83 | 2-thienyl | H |
| 84 | 3-thienyl | H |
| 85 | 2-furyl | H |
| 86 | 3-furyl | H |
| 87 | NH₂ | H |
| 88 | OCH₃ | H |
| 89 | H | CH₃ |
| 90 | CH₃ | CH₃ |
| 91 | CH₂CH₃ | CH₃ |
| 92 | (CH₂)₂CH₃ | CH₃ |
| 93 | (CH₂)₃CH₃ | CH₃ |
| 94 | (CH₂)₄CH₃ | CH₃ |
| 95 | (CH₂)₅CH₃ | CH₃ |
| 96 | CH(CH₃)₂ | CH₃ |
| 97 | CH(CH₃)CH₂CH₃ | CH₃ |
| 98 | CH₂CH(CH₃)₂ | CH₃ |
| 99 | C(CH₃)₃ | CH₃ |
| 100 | C(CH₃)₂CH₂CH₃ | CH₃ |
| 101 | CH₂C(CH₃)₃ | CH₃ |
| 102 | CH₂-cyclopropyl | CH₃ |
| 103 | CH(CH₃)-cyclopropyl | CH₃ |
| 104 | CH₂-cyclobutyl | CH₃ |
| 105 | CH₂-cylopentyl | CH₃ |
| 106 | CH₂-cyclohexyl | CH₃ |
| 107 | CH₂OCH₃ | CH₃ |
| 108 | CH₂CH₂OCH₃ | CH₃ |
| 109 | CF₃ | CH₃ |
| 110 | CH₂NH₂ | CH₃ |
| 111 | CH₂SCH₃ | CH₃ |
| 112 | CH₂SOCH₃ | CH₃ |
| 113 | CH₂SO₂CH₃ | CH₃ |
| 114 | cyclopropyl | CH₃ |
| 115 | cyclobutyl | CH₃ |
| 116 | cyclopentyl | CH₃ |
| 117 | cyclohexyl | CH₃ |
| 118 | CH₂CH═CH₂ | CH₃ |
| 119 | CH(CH₃)CH═CH₂ | CH₃ |
| 120 | CH₂CHCH═CH₃ | CH₃ |
| 121 | CH₂C≡CH | CH₃ |
| 122 | C(CH₃)C≡CH | CH₃ |
| 123 | C₆H₅ | CH₃ |
| 124 | 2-Cl—C₆H₄ | CH₃ |
| 125 | 3-Cl—C₆H₄ | CH₃ |
| 126 | 4-Cl—C₆H₄ | CH₃ |
| 127 | 3,4-Cl₂—C₆H₄ | CH₃ |
| 128 | 2,4-Cl₂—C₆H₄ | CH₃ |
| 129 | 2,6-Cl₂—C₆H₄ | CH₃ |
| 130 | 2,3-Cl₂—C₆H₄ | CH₃ |
| 131 | 3,5-Cl₂—C₆H₄ | CH₃ |
| 132 | 2-(CH₃)—C₆H₄ | CH₃ |
| 133 | 3-(CH₃)—C₆H₄ | CH₃ |
| 134 | 4-(CH₃)—C₆H₄ | CH₃ |
| 135 | 2-(OCH₃)—C₆H₄ | CH₃ |
| 136 | 3-(OCH₃)—C₆H₄ | CH₃ |
| 137 | 4-(OCH₃)—C₆H₄ | CH₃ |
| 138 | 2,6-Cl₂—C₆H₃ | CH₃ |
| 139 | 2,6-(CH₃)₂—C₆H₃ | CH₃ |
| 140 | 2,3-(CH₃O)₂—C₆H₃ | CH₃ |
| 141 | 2,4-(CH₃O)₂—C₆H₃ | CH₃ |
| 142 | 3,4-(CH₃O)₂—C₆H₃ | CH₃ |
| 143 | 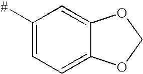 | CH₃ |
| 144 | 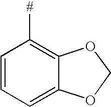 | CH₃ |
| 145 | 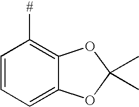 | CH₃ |
| 146 | 2-(NO₂)—C₆H₄ | CH₃ |
| 147 | 3-(NO₂)—C₆H₄ | CH₃ |
| 148 | 4-(NO₂)—C₆H₄ | CH₃ |
| 149 | 2-F—C₆H₄ | CH₃ |
| 150 | 3-F—C₆H₄ | CH₃ |
| 151 | 4-F—C₆H₄ | CH₃ |
| 152 | 2-Br—C₆H₄ | CH₃ |
| 153 | 3-Br—C₆H₄ | CH₃ |
| 154 | 4-Br—C₆H₄ | CH₃ |
| 155 | 2-(CF₃)—C₆H₄ | CH₃ |
| 156 | 3-(CF₃)—C₆H₄ | CH₃ |
| 157 | 4-(CF₃)—C₆H₄ | CH₃ |
| 158 | 2-(OCF₃)—C₆H₄ | CH₃ |
| 159 | 3-(OCF₃)—C₆H₄ | CH₃ |
| 160 | 4-(OCF₃)—C₆H₄ | CH₃ |
| 161 | 4-(H₃C—SO₂)—C₆H₄ | CH₃ |
| 162 | 2-NC—C₆H₄ | CH₃ |
| 163 | 3-NC—C₆H₄ | CH₃ |
| 164 | 4-NC—C₆H₄ | CH₃ |
| 165 | CH₂—C₆H₅ | CH₃ |
| 166 | 1-naphthyl | CH₃ |
| 167 | 2-naphthyl | CH₃ |
| 168 | 2-pyridyl | CH₃ |
| 169 | 3-pyridyl | CH₃ |
| 170 | 4-pyridyl | CH₃ |
| 171 | 2-thienyl | CH₃ |
| 172 | 3-thienyl | CH₃ |
| 173 | 2-furyl | CH₃ |
| 174 | 3-furyl | CH₃ |
| 175 | NH₂ | CH₃ |
| 176 | OCH₃ | CH₃ |

Examples of preferred compounds Ib, Ic, Ib-R, Ic-R, Ib-S and Ic-S (i.e. compounds I, I-R and I-S, wherein R¹ is as defined form Ib or Ic, respectively) are given in the following tables 33 to 64.

### Table 33

Compounds of the formulae Ib, Ic, Ib-R, Ib-S, Ic-R and Ic-S, wherein R³ and R⁴ are hydrogen, n is 1 and the combination of R¹ and R² for one compound corresponds in each case to one row of table B.

### Table 34

Compounds of the formulae Ib, Ic, Ib-R, Ib-S, Ic-R and Ic-S, wherein R³ is hydrogen and R⁴ is chlorine, n is 1 and the combination of R¹ and R² for one compound corresponds in each case to one row of table B.

### Table 35

Compounds of the formulae Ib, Ic, Ib-R, Ib-S, Ic-R and Ic-S, wherein R³ is hydrogen and R⁴ is bromine, n is 1 and the combination of R¹ and R² for one compound corresponds in each case to one row of table B.

### Table 36

Compounds of the formulae Ib, Ic, Ib-R, Ib-S, Ic-R and Ic-S, wherein R³ is hydrogen and R⁴ is fluorine, n is 1 and the combination of R¹ and R² for one compound corresponds in each case to one row of table B.

### Table 37

Compounds of the formulae Ib, Ic, Ib-R, Ib-S, Ic-R and Ic-S, wherein R³ is chlorine and R⁴ is hydrogen, n is 1 and the combination of R¹ and R² for one compound corresponds in each case to one row of table B.

### Table 38

Compounds of the formulae Ib, Ic, Ib-R, Ib-S, Ic-R and Ic-S, wherein R³ is chlorine and R⁴ is chlorine, n is 1 and the combination of R¹ and R² for one compound corresponds in each case to one row of table B.

### Table 39

Compounds of the formulae Ib, Ic, Ib-R, Ib-S, Ic-R and Ic-S, wherein R³ is chlorine and R⁴ is bromine, n is 1 and the combination of R¹ and R² for one compound corresponds in each case to one row of table B.

### Table 40

Compounds of the formulae Ib, Ic, Ib-R, Ib-S, Ic-R and Ic-S, wherein R³ is chlorine and R⁴ is fluorine, n is 1 and the combination of R¹ and R² for one compound corresponds in each case to one row of table B.

### Table 41

Compounds of the formulae Ib, Ic, Ib-R, Ib-S, Ic-R and Ic-S, wherein R³ is fluorine and R⁴ is hydrogen, n is 1 and the combination of R¹ and R² for one compound corresponds in each case to one row of table B.

### Table 42

Compounds of the formulae Ib, Ic, Ib-R, Ib-S, Ic-R and Ic-S, wherein R³ is fluorine and R⁴ is chlorine, n is 1 and the combination of R¹ and R² for one compound corresponds in each case to one row of table B.

### Table 43

Compounds of the formulae Ib, Ic, Ib-R, Ib-S, Ic-R and Ic-S, wherein R³ is fluorine and R⁴ is bromine, n is 1 and the combination of R¹ and R² for one compound corresponds in each case to one row of table B.

### Table 44

Compounds of the formulae Ib, Ic, Ib-R, Ib-S, Ic-R and Ic-S, wherein R³ is fluorine and R⁴ is fluorine, n is 1 and the combination of R¹ and R² for one compound corresponds in each case to one row of table B.

### Table 45

Compounds of the formulae Ib, Ic, Ib-R, Ib-S, Ic-R and Ic-S, wherein R³ is bromine and R⁴ is hydrogen, n is 1 and the combination of R¹ and R² for one compound corresponds in each case to one row of table B.

### Table 46

Compounds of the formulae Ib, Ic, Ib-R, Ib-S, Ic-R and Ic-S, wherein R³ is bromine and R⁴ is chlorine, n is 1 and the combination of R¹ and R² for one compound corresponds in each case to one row of table B.

### Table 47

Compounds of the formulae Ib, Ic, Ib-R, Ib-S, Ic-R and Ic-S, wherein R³ is bromine and R⁴ is bromine, n is 1 and the combination of R¹ and R² for one compound corresponds in each case to one row of table B.

### Table 48

Compounds of the formulae Ib, Ic, Ib-R, Ib-S, Ic-R and Ic-S, wherein R³ is bromine and R⁴ is fluorine, n is 1 and the combination of R¹ and R² for one compound corresponds in each case to one row of table B.

### Table 49

Compounds of the formulae Ib, Ic, Ib-R, Ib-S, Ic-R and Ic-S, wherein R³ and R⁴ are hydrogen, n is 2 and the combination of R¹ and R² for one compound corresponds in each case to one row of table B.

### Table 50

Compounds of the formulae Ib, Ic, Ib-R, Ib-S, Ic-R and Ic-S, wherein R³ is hydrogen and R⁴ is chlorine, n is 2 and the combination of R¹ and R² for one compound corresponds in each case to one row of table B.

### Table 51

Compounds of the formulae Ib, Ic, Ib-R, Ib-S, Ic-R and Ic-S, wherein R³ is hydrogen and R⁴ is bromine, n is 2 and the combination of R¹ and R² for one compound corresponds in each case to one row of table B.

### Table 52

Compounds of the formulae Ib, Ic, Ib-R, Ib-S, Ic-R and Ic-S, wherein R³ is hydrogen and R⁴ is fluorine, n is 2 and the combination of R¹ and R² for one compound corresponds in each case to one row of table B.

### Table 53

Compounds of the formulae Ib, Ic, Ib-R, Ib-S, Ic-R and Ic-S, wherein R³ is chlorine and R⁴ is hydrogen, n is 2 and the combination of R¹ and R² for one compound corresponds in each case to one row of table B.

### Table 54

Compounds of the formulae Ib, Ic, Ib-R, Ib-S, Ic-R and Ic-S, wherein R³ is chlorine and R⁴ is chlorine, n is 2 and the combination of R¹ and R² for one compound corresponds in each case to one row of table B.

### Table 55

Compounds of the formulae Ib, Ic, Ib-R, Ib-S, Ic-R and Ic-S, wherein R³ is chlorine and R⁴ is bromine, n is 2 and the combination of R¹ and R² for one compound corresponds in each case to one row of table B.

### Table 56

Compounds of the formulae Ib, Ic, Ib-R, Ib-S, Ic-R and Ic-S, wherein R³ is chlorine and R⁴ is fluorine, n is 2 and the combination of R¹ and R² for one compound corresponds in each case to one row of table B.

### Table 57

Compounds of the formulae Ib, Ic, Ib-R, Ib-S, Ic-R and Ic-S, wherein R³ is fluorine and R⁴ is hydrogen, n is 2 and the combination of R¹ and R² for one compound corresponds in each case to one row of table B.

### Table 58

Compounds of the formulae Ib, Ic, Ib-R, Ib-S, Ic-R and Ic-S, wherein R³ is fluorine and R⁴ is chlorine, n is 2 and the combination of R¹ and R² for one compound corresponds in each case to one row of table B.

### Table 59

Compounds of the formulae Ib, Ic, Ib-R, Ib-S, Ic-R and Ic-S, wherein R³ is fluorine and R⁴ is bromine, n is 2 and the combination of R¹ and R² for one compound corresponds in each case to one row of table B.

### Table 60

Compounds of the formulae Ib, Ic, Ib-R, Ib-S, Ic-R and Ic-S, wherein R³ is fluorine and R⁴ is fluorine, n is 2 and the combination of R¹ and R² for one compound corresponds in each case to one row of table B.

### Table 61

Compounds of the formulae Ib, Ic, Ib-R, Ib-S, Ic-R and Ic-S, wherein R³ is bromine and R⁴ is hydrogen, n is 2 and the combination of R¹ and R² for one compound corresponds in each case to one row of table B.

### Table 62

Compounds of the formulae Ib, Ic, Ib-R, Ib-S, Ic-R and Ic-S, wherein R³ is bromine and R⁴ is chlorine, n is 2 and the combination of R¹ and R² for one compound corresponds in each case to one row of table B.

### Table 63

Compounds of the formulae Ib, Ic, Ib-R, Ib-S, Ic-R and Ic-S, wherein R³ is bromine and R⁴ is bromine, n is 2 and the combination of R¹ and R² for one compound corresponds in each case to one row of table B.

### Table 64

Compounds of the formulae Ib, Ic, Ib-R, Ib-S, Ic-R and Ic-S, wherein R³ is bromine and R⁴ is fluorine, n is 2 and the combination of R¹ and R² for one compound corresponds in each case to one row of table B.

TABLE B

| | R¹ | R² |
|---|---|---|
| 1 | H | H |
| 2 | CH₃ | H |
| 3 | CH₂CH₃ | H |
| 4 | (CH₂)₂CH₃ | H |
| 5 | (CH₂)₃CH₃ | H |
| 6 | (CH₂)₄CH₃ | H |
| 7 | (CH₂)₅CH₃ | H |
| 8 | CH(CH₃)₂ | H |
| 9 | CH(CH₃)CH₂CH₃ | H |
| 10 | CH₂CH(CH₃)₂ | H |
| 11 | C(CH₃)₃ | H |
| 12 | C(CH₃)₂CH₂CH3 | H |
| 13 | CH₂C(CH₃)₃ | H |
| 14 | CH₂-cyclopropyl | H |
| 15 | CH(CH₃)-cyclopropyl | H |
| 16 | CH₂-cyclobutyl | H |
| 17 | CH₂-cylopentyl | H |
| 18 | CH₂-cyclohexyl | H |
| 19 | CH₂OCH₃ | H |
| 20 | CH₂CH₂OCH₃ | H |
| 21 | CF₃ | H |
| 22 | CH₂NH₂ | H |
| 23 | CH₂SCH₃ | H |
| 24 | CH₂SOCH₃ | H |
| 25 | CH₂SO₂CH₃ | H |
| 26 | cyclopropyl | H |
| 27 | cyclobutyl | H |
| 28 | cyclopentyl | H |
| 29 | cyclohexyl | H |
| 30 | CH₂CH═CH₂ | H |
| 31 | CH(CH₃)CH═CH₂ | H |
| 32 | CH₂CHCH═CH₃ | H |
| 33 | CH₂C≡CH | H |
| 34 | C(CH₃)C≡CH | H |
| 35 | C₆H₅ | H |
| 36 | 2-Cl—C₆H₄ | H |
| 37 | 3-Cl—C₆H₄ | H |
| 38 | 4-Cl—C₆H₄ | H |
| 39 | 2-(CH₃)—C₆H₄ | H |
| 40 | 3-(CH₃)—C₆H₄ | H |
| 41 | 4-(CH₃)—C₆H₄ | H |
| 42 | 2-(OCH₃)—C₆H₄ | H |
| 43 | 3-(OCH₃)—C₆H₄ | H |
| 44 | 4-(OCH₃)—C₆H₄ | H |
| 45 | 2,6-Cl₂—C₆H₃ | H |
| 46 | 2,6-(CH₃)₂—C₆H₃ | H |
| 47 | 2,6-(CH₃O)₂—C₆H₃ | H |
| 48 | CH₂—C₆H₅ | H |
| 49 | NH₂ | H |
| 50 | OCH₃ | H |
| 51 | H | CH₃ |
| 52 | CH₃ | CH₃ |
| 53 | CH₂CH₃ | CH₃ |
| 54 | (CH₂)₂CH₃ | CH₃ |
| 55 | (CH₂)₃CH₃ | CH₃ |
| 56 | (CH₂)₄CH₃ | CH₃ |
| 57 | (CH₂)₅CH₃ | CH₃ |
| 58 | CH(CH₃)₂ | CH₃ |
| 59 | CH(CH₃)CH₂CH₃ | CH₃ |
| 60 | CH₂CH(CH₃)₂ | CH₃ |
| 61 | C(CH₃)₃ | CH₃ |
| 62 | C(CH₃)₂CH₂CH₃ | CH₃ |
| 63 | CH₂C(CH₃)₃ | CH₃ |
| 64 | CH₂-cyclopropyl | CH₃ |
| 65 | CH(CH₃)-cyclopropyl | CH₃ |
| 66 | CH₂-cyclobutyl | CH₃ |
| 67 | CH₂-cylopentyl | CH₃ |
| 68 | CH₂-cyclohexyl | CH₃ |
| 69 | CH₂OCH₃ | CH₃ |
| 70 | CH₂CH₂OCH₃ | CH₃ |
| 71 | CF₃ | CH₃ |
| 72 | CH₂NH₂ | CH₃ |
| 73 | CH₂SCH₃ | CH₃ |
| 74 | CH₂SOCH₃ | CH₃ |
| 75 | CH₂SO₂CH₃ | CH₃ |
| 76 | cyclopropyl | CH₃ |
| 77 | cyclobutyl | CH₃ |
| 78 | cyclopentyl | CH₃ |
| 79 | cyclohexyl | CH₃ |
| 80 | CH₂CH═CH₂ | CH₃ |
| 81 | CH(CH₃)CH═CH₂ | CH₃ |
| 82 | CH₂CHCH═CH₃ | CH₃ |
| 83 | CH₂C≡CH | CH₃ |
| 84 | C(CH₃)C≡CH | CH₃ |
| 85 | CH₂—C₆H₅ | CH₃ |

Likewise, compounds of the formula II are preferred, wherein, independently of each other,

R² is H or methyl,

R³ is hydrogen or halogen, in particular fluorine, chlorine or bromine, and

R⁴ is hydrogen or halogen, in particular fluorine, chlorine or bromine.

Preferably at least one radical R³ or R⁴ is different from hydrogen.

Likewise, compounds of the formula IIa are preferred, wherein, independently of each other,

R^{2a} is methyl,

R^{3a} is hydrogen or halogen, in particular fluorine, chlorine or bromine, and

R^{4a} is hydrogen or halogen, in particular fluorine, chlorine or bromine.

Preferably at least one radical R^{3a} or R^{4a} is different from hydrogen.

Examples of preferred compounds II (i.e. compounds II, II-R and II-S) are given in the following tables 65 to 68.

### Table 65

Compounds of the formulae II, II-R and II-S, wherein R² is hydrogen, n is 1 and the combination of R³ and R⁴ for one compound corresponds in each case to one row of table C.

### Table 66

Compounds of the formulae II, II-R and II-S, wherein R² is methyl, n is 1 and the combination of R³ and R⁴ for one compound corresponds in each case to one row of table C. These compounds correspond to compounds of the formula IIa, wherein R² is methyl and wherein the combination of R³ and R⁴ for one compound corresponds in each case to one row of table C.

### Table 67

Compounds of the formulae II, II-R and II-S, wherein R² is hydrogen, n is 2 and the combination of R³ and R⁴ for one compound corresponds in each case to one row of table C.

### Table 68

Compounds of the formulae II, II-R and II-S, wherein R² is methyl, n is 2 and the combination of R³ and R⁴ for one compound corresponds in each case to one row of table C.

TABLE C

| Compound II | R³ | R⁴ |
|---|---|---|
| 1 | H | H |
| 2 | Cl | H |
| 3 | Br | H |
| 4 | F | H |
| 5 | H | Cl |
| 6 | H | Br |
| 7 | H | F |
| 8 | Cl | Cl |
| 9 | Cl | F |
| 10 | Cl | Br |
| 11 | F | Cl |
| 12 | F | F |
| 13 | F | Br |
| 14 | Br | Cl |
| 15 | Br | F |
| 16 | Br | Br |

The compounds of the general formulae I can be synthesized by techniques generally known in the art. A synthetic route to compounds of the formula I is shown in scheme 1. In scheme 1, n, R, R², R³ and R⁴ are as defined above. X is a leaving group, which can be replaced by amine nucleophiles. Examples of X are halogen, C₁-C₄-alkoxy and the like. R^{x} is e.g. C₁-C₆-alkyl, C₂-C₆-alkenyl, C₂-C₆-alkynyl, C₃-C₆-cycloalkyl, C₃-C₆-cycloalkyl-C₁-C₄-alkyl, or phenyl or substituted phenyl, in particular C₁-C₄-alkyl.

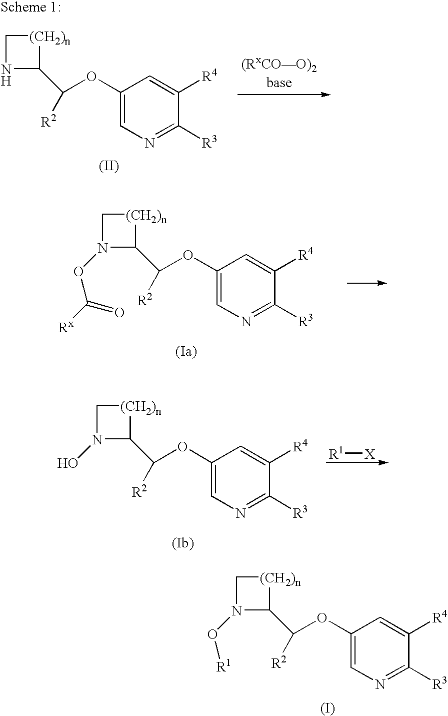

In the first step, a pyridine compound of the formula II is oxidized with the anhydride of a peracid (compound R^{x}CO—O)₂) in the presence of a base to obtain a compound of the formula Ia.

The oxidation in the first step of scheme 1 can be accomplished by the use of a peracid derivatives (e.g. anhydrides of those) in inert organic solvents in the presence of a base. Suitable peracid anhydrides are (C₆H₅COO)₂, (3-C₁-C₆H₄COO)₂ or (CH₃COO)₂. However, other peracid anhydrides (R^{x}COO)₂, can also be used. Suitable bases comprise hydroxides, carbonates, hydrogen carbonates, hydrogen phosphates and dihydrogen phosphates of alkali metals such as NaOH, KOH, NaHCO₃, KHCO₃, Na₂CO₃, K₂CO₃, NaH₂PO₄, Na₂HPO₄, KH₂PO₄, K₂HPO₄, and also organic bases, in particular tertiary amines like pyridine compounds and trialkylamines, e.g. pyridine triethylamine, tributylamine or preferably polymer bound tertiary amine bases like poly-4-vinylpyridine.

Preferably oxidation of II is performed in an inert solvent. Inert solvents comprise e.g. aromatic hydrocarbons such as benzene, toluene or xylene, cyclic ethers such as tetrahydrofurane, chlorinated hydrocarbons such as dichloromethane, trichloromethane, dichloroethane, and the mixtures thereof or the mixtures thereof with aliphatic or cycloaliphatic hydrocarbons such as hexane, cyclohexane and the like. Preferred solvent is dichloromethane. In some cases it might be beneficial to run the oxidation in a 2 phase system comprising water and a halogenated solvent such as dichloromethane or water and aromatic solvent such as toluene or the like.

The oxidation is usually run in a temperature range from 0 to 100° C., preferably from 20 to 90° C.

For details see e.g. Org. Synth. 1984 62, 149, J. Org. Chem. 1981 46 (10) 2148, J. Org. Chem. 1998 63 (5) 1491, J. Org. Chem. 1992 57(26) 7140, J. Org. Chem. 2000 (65 (5) 1442, Synthesis 1983 7 537, J. Am. Chem. Soc. 1992 114 (1) 383, Tetrahedron Lett. 1989 30 (47) 6491, Angew. Che, Int. Ed. 2002 41(24) 4688, Houben Weyl (Thieme 1990) Vol. E16a, p 84 ff and Vol. 10/1 p. 1135.

The thus obtained compound Ia with R⁵=R^{x} is then cleaved under saponification conditions to obtain a compound of the formula I, wherein R¹ is hydrogen. Appropriate conditions for saponification are known to those skilled and can be found in Synthesis 2001, 11, p. 1611, Synthesis 1983, p. 537, J. Org. Chem. 1981, p. 2148 and Chem. Ber. 1919, 52, 1667.

Thus obtained compound Ib can be acylated with a compound R⁵—CO—X, wherein R⁵ and X are as defined above, with preference given to X being halogen, in particular chlorine. The acylation of Ib can be performed by analogy with conventional acylation reactions of hydroxy compounds. For details see e.g. J. Org. Chem. 1987 52 3457, Tetrahedron Lett. 1989, p. 6491, Angew. Chem. Int. Ed. 2002, 41 (24), p. 4698.

Preferably the acylation of compounds Ib is performed in the presence of a base such as tertiary amines and pyridines such as triethylamine, diethylisopropylamin, pyridine, alkalimetal carbonates and hydrogen carbonates such as lithium carbonate, sodium carbonate, sodium hydrogencarbonate, potassium hydrogencarbonate or K₂CO₃. Preferably the acylation is performed in an inert solvent such as aromatic hydrocarbons, e.g. benzene, toluene or xylene, cyclic ethers such as tetrahydrofurane, chlorinated hydrocarbons such as dichloromethane, trichloromethane, dichloroethane, and the mixtures thereof or the mixtures thereof with aliphatic or cycloaliphatic hydrocarbons such as hexane, cyclohexane and the like.

Likewise, compounds of the formula Ib can be alkylated with a suitable alkylation reagent R¹X, wherein R¹ is as defined for compounds Ic or Id and X is a suitable leaving group.

Compounds wherein R¹ is C(O)NH₂ can be obtained by reacting the compound Ib with alkalimetal cyanate such as potassium cyanate (KOCN) in the presence of an acid, such as hydrochloric acid.

Pyridine N-oxides of I can be obtained according to the following reaction scheme 2 by oxidizing compounds of the general formula I with suitable oxidizing agents such as organic peracids (for details see Katritzky et al. “Chemistry of Heterocyclic N-oxides pp. 21-72, 539-542, Academic Press, New York 1971 and the literatur cited therein).

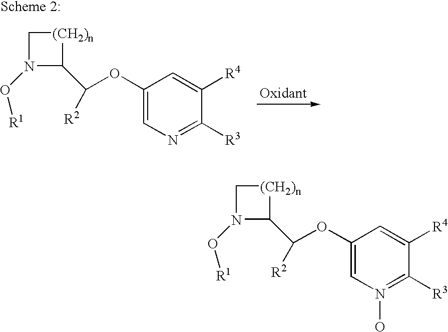

Compounds of the general formulae II and III are known in the art, e.g. from U.S. Pat. No. 5,629,325, U.S. Pat. No. 5,948,793, U.S. Pat. No. 6,127,386, WO 9640682, WO 97/46554, U.S. Pat. No. 6,437,138, WO9932480, WO9825920 or WO 9408992 or can be prepared by analogy to the methods described therein.

Compounds of the formula IIa can be prepared e.g. by the route outlined in scheme 3:

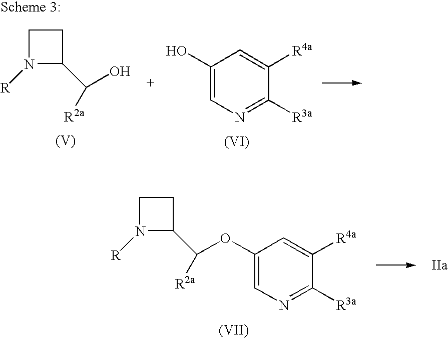

In scheme 3, n, R^{2a}, R^{3a} and R^{4a} are as defined above. R is conventional protecting group, e.g. an urethane protecting group such as BOC or CBZ. According to the method of scheme 3, a hydroxymethyl compound of the general formula V is reacted with 3-hydroxypyridines of the formula VI under Mitsunobu conditions, e.g. in the presence of a triaryl phosphine compound such as triphenyl phosphine in the presence of a azodicarboxylate ester such as diethyl azodicarboxylate (DEAD). Thereby compounds of the formula VII are obtained, which can be deprotected to obtain compounds of the formula IIa. The reaction can be performed by analogy to conventional Mitsunobu reaction as described in Org. React. 1992, 42, pp. 335-656, Synthesis 1981, pp. 1-28, J. Med. Chem. 1996, 39, pp. 817-825. Pyridine N-oxides of IIa can be obtained by oxidizing compounds of the general formula VII by suitable oxidizing agents such as organic peracids (for details see Katritzky et al. “Chemistry of Heterocyclic N-oxides pp. 21-72, 539-542, Academic Press, New York 1971 and the literatur cited therein) and therafter removing the protective group R.

The compounds V can be synthesized according to know procedures, for details see Tetrahedron Lett. 42 2001 8247, Tetrahedron lett 30 11 1331, Tetrahedron Lett. 30 11 1327 1989, Tetrahedron, Asym., 9, 1998, 1935 and Houben Weyl (Thieme 1990) Vol. E16a, p 84 ff and p 304 ff.

Work-up of the reaction mixture to isolate compounds I or IIa, respectively, can be performed by conventional processes, including filtration, extraction and solvent removing. Purification can be achieved e.g. by crystallization and/or chromatography.

Some of the intermediates and end products are obtained in the form of colourless or slightly brownish viscous oils which can be purified or freed from volatile components under reduced pressure and at moderately elevated temperature. If the intermediates and the end products are obtained as solids, purification can also be carried out by recrystallization or digestion.

If individual compounds I or IIa can not be obtained by the routes described above, they can be prepared by derivatization of other compounds I or IIa.

If the synthesis yields a mixtures of isomers, a separation of the isomers is possible. However a separation is generally not necessary. In some cases the individual isomers will be interconverted during work-up for use or during application (for example under the action of light, acids or bases). Such conversions may also take place after use.

The compounds of the formulae I and II, their pyridine N-oxides as well as the salts thereof are in particular suitable for efficiently controlling arthropodal pests such as arachnids and insects as well as nematodes.

In particular, they are suitable for controlling insect pests, such as insects from the order of the

lepidopterans (Lepidoptera), for example Agrotis ypsilon, Agrotis segetum, Alabama argillacea, Anticarsia gemmatalis, Argyresthia conjugella, Autographa gamma, Bupalus piniarius, Cacoecia murinana, Capua reticulana, Chematobia brumata, Choristoneura fumiferana, Choristoneura occidentals, Cirphis unipuncta, Cydia pomonella, Dendrolimus pini Diaphania nitidalis, Diatraea grandiosella, Earias insulana, Elasmopalpus lignosellus, Eupoecilia ambiguella, Evetria bouliana, Feltia subterranea, Galleria mellonella, Grapholltha funebrana, Grapholitha molesta, Hellothis armigera, Hellothis virescens, Hellothis zea, Hellula undalls, Hibernia defoliaria, Hyphantria cunea, Hyponomeuta malinellus, Keiferia lycopersicella, Lambdina fiscellaria, Laphygma exigua, Leucoptera coffeella, Leucoptera scitella, Lithocolletis blancardella, Lobesia botrana, Loxostege sticticalls, Lymantria dispar, Lymantria monacha, Lyonetia clerkella, Malacosoma neustria, Mamestra brassicae, Orgyia pseudotsugata, Ostrinia nubilalls, Panolis flammea, Pectinophora gossypiella, Peridroma saucia, Phalera bucephala, Phthorimaea operculella, Phyllocnistis citrella, Pieris brassicae, Plathypena scabra, Plutella xylostella, Pseudoplusia includens, Rhyacionia frustrana, Scrobipalpula absoluta, Sitotroga cerealella, Sparganothis pilleriana, Spodoptera frugiperda, Spodoptera littoralls, Spodoptera litura, Thaumatopoea pityocampa, Tortrix viridana, Trichoplusia ni and Zeiraphera canadensis,

beetles (Coleoptera), for example Agrilus sinuatus, Agriotes lineatus, Agriotes obscurus, Amphimallus solstltialis, Anisandrus dispar, Anthonomus grandis, Anthonomus pomorum, Aphthona euphoridae, Athous haemorrhoidalls, Atomaria linearis, Blastophagus piniperda, Blitophaga undata, Bruchus rurimanus, Bruchus pisorum, Bruchus lentis, Byctiscus betulae, Cassida nebulosa, Cerotoma trifurcata, Cetonia aurata, Ceuthorrhynchus asimilis, Ceuthorrhynchus napi, Chaetocnema tibialis, Conoderus vespertinus, Crioceris asparagi, Ctenicera ssp., Diabrotica longicornis, Diabrotica semipunctata, Diabrotica 12-punctata Diabrotica speciosa, Diabrotica virgifera, Epilachna varivestis, Epitrix hirtipennis, Eutinobothrus brasillensis, Hylobius abietis, Hypera brunneipennis, Hypera postica, Ips typographus, Lema blilneata, Lema melanopus, Leptinotarsa decemlineata, Limonius californicus, Lissorhoptrus oryzophilus, Melanotus communis, Meligethes aeneus, Melolontha hippocastani, Melolontha melolontha, Oulema oryzae, Ortiorrhynchus sulcatus, Otiorrhynchus ovatus, Phaedon cochleariae, Phyllobius pyri, Phyllotreta chrysocephala, Phyllophaga sp., Phyllopertha horticola, Phyllotreta nemorum, Phyllotreta striolata, Poplilla japonica, Sitona lineatus and Sitophilus granaria,

flies, mosquitoes (Diptera), e.g. Aedes aegypti, Aedes albopictus, Aedes vexans, Anastrepha ludens, Anopheles maculipennis, Anopheles crucians, Anopheles albimanus, Anopheles gambiae, Anopheles freeborni, Anopheles leucosphyrus, Anopheles minimus, Anopheles quadrimaculatus, Calliphora vicina, Ceratitis capitata, Chrysomya bezziana, Chrysomya hominivorax, Chrysomya macellaria, Chrysops discalis, Chrysops silacea, Chrysops atlanticus, Cochliomyia hominivorax, Contarinia sorghicola Cordylobia anthropophaga, Culicoides furens, Culex pipiens, Culex nigripalpus, Culex quinquefasciatus, Culex tarsalls, Culiseta inornata, Culiseta melanura, Dacus cucurbitae, Dacus oleae, Dasineura brassicae, Della antique, Delia coarctata, Della platura, Della radicum, Dermatobia homrnis, Fannia canicularis, Geomyza Tripunctata, Gasterophilus intestinalls, Glossina morsitanis, Glossina palpalls, Glossina fuscipes, Glossina tachinoides, Haematobia irritans, Haplodiplosis equestris, Hippelates spp., Hylemyia platura, Hypoderma lineata, Leptoconops torrens, Liriomyza sativae, Liriomyza trifolii, Lucilia caprina, Lucilla cuprina, Lucilia sericata, Lycoria pectoralls, Mansonia titillanus, Mayetiola destructor, Musca domestica, Muscina stabulans, Oestrus ovis, Opomyza florum, Oscinella frit, Pegomya hysocyami, Phorbia antiqua, Phorbia brassicae, Phorbia coarctata, Phlebotomus argentipes, Psorophora columbiae, Psila rosae, Psorophora discolor, Prosimullum mixtum, Rhagoletis cerasi, Rhagoletis pomonella, Sarcophaga haemorrhoidalis, Sarcophaga sp., Simulium vittatum, Stomoxys caIcitrans, Tabanus bovinus, Tabanus atratus, Tabanus lineola, and Tabanus similis, Tipula oleracea, and Tipula paludosa

thrips (Thysanoptera), e.g. Dichromothrips corbetti, Dichromothrips ssp, Frankliniella fusca, Frankliniella occidentalls, Frankliniella tritici, Scirtothrips citri, Thrips oryzae, Thrips palmiand Thrips tabaci,

termites (Isoptera), e.g. Calotermes flavicollis, Leucotermes flavipes, Heterotermes aureus, Reticulitermes flavlpes, Reticulitermes virginicus, Reticulitermes lucifugus, Termes natalensis, and Coptotermes formosanus,

cockroaches (Blattaria—Blattodea), e.g. Blaftella germanica, Blattella asahinae, Periplaneta americana, Periplaneta japonica, Periplaneta brunnea, Periplaneta fuligginosa, Periplaneta australasiae, and Blafta orientalis,

true bugs (Hemiptera), including Heteroptera, such as Acrosternum hilare, Blissus leucopterus, Cyrtopeltis notatus, Dysdercus cingulatus, Dysdercus intermedius, Eurygaster integriceps, Euschistus impictiventris, Leptoglossus phyllopus, Lygus lineolaris, Lygus pratensis, Nezara viridula, Piesma quadrata, Solubea insularis and Thyanta perditor, including also Homoperta such as Acyrthosiphon onobrychis, Adelges laricis, Aphidula nasturtii, Aphis fabae, Aphis forbesi, Aphis pomi, Aphis gossypii, Aphis grossulariae, Aphis schneideri, Aphis spiraecola, Aphis sambuci, Acyrthosiphon pisum, Aulacorthum solani, Bemisia argentifolii, Brachycaudus cardui, Brachycaudus helichrysi, Brachycaudus persicae, Brachycaudus prunicola, Brevicoryne brassicae, Capitophorus horni, Cerosipha gossypi, Chaetosiphon fragaefolil, Cryptomyzus ribis, Dreyfusia nordmannianae, Dreyfusia piceae, Dysaphis radicola, Dysaulacorthum pseudosolani, Dysaphis plantaginea, Dysaphis pyri, Empoasca fabae, Hyalopterus pruni, Hyperomyzus lactucae, Macrosiphum avenae, Macrosiphum euphorbiae, Macrosiphon rosae, Megoura viciae, Melanaphis pyrarius, Metopolophium dirhodum, Myzus persicae, Myzus ascalonicus, Myzus cerasi, Myzus varians, Nasonovia ribis-nigr, Nilaparvata lugens, Pemphigus bursarius, Perkinsiella saccharicida, Phorodon humuli, Psylla mali, Psylla piri, Rhopalomyzus ascalonicus, Rhopalosiphum maidis, Rhopalosiphum padi, Rhopalosiphum insertum, Sappaphis mala, Sappaphis mali, Schizaphis graminum, Schizoneura lanuginosa, Sitobion avenae, Trialeurodes vaporariorum, Toxoptera aurantlland, Viteus vitifolii, Cimex lectularius, Cimex hemipterus, Reduvius senilis, Triatoma spp., and Arilus critatus.

ants, bees, wasps, sawflies (Hymenoptera), e.g. Athalia rosae, Atta cephalotes, Atta capiguara, Atta cephalotes, Atta laevigata, Atta robusta, Atta sexdens, Atta texana, Crematogaster spp., Hoplocampa minuta, Hoplocampa testudinea, Monomorium pharaonis, Solenopsis geminata, Solenopsis invicta, Solenopsis richteri, Solenopsis xyloni, Pogonomyrmex barbatus, Pogonomyrmex californicus, Pheidole megacephala, Dasymutilla occidentalis, Bombus spp. Vespula squamosa, Paravespula vulgaris, Paravespula pennsylvanica, Paravespula germanica, Dolichovespula maculata, Vespa crabro, Polistes rubiginosa, Camponotus floridanus, and Linepithema humile,

crickets, grasshoppers, locusts (Orthoptera), e.g. Acheta domestica, Gryllotalpa gryllotalpa, Locusta migratoria, Melanoplus bivittatus, Melanoplus femurrubrum, Melanoplus mexicanus, Melanoplus sanguinipes, Melanoplus spretus, Nomadacris septemfasciata, Schistocerca americana, Schistocerca gregaria, Dociostaurus maroccanus, Tachycines asynamorus, Oedaleus senegalensis, Zonozerus variegatus, Hieroglyphus daganensis, Kraussaria angulifera, Calliptamus italicus, Chortoicetes terminifera, and Locustana pardalina,

fleas (Siphonaptera), e.g. Ctenocephalides felis, Ctenocephalides canis, Xenopsylla cheopis, Pulex irritans, Tunga penetrans, and Nosopsyllus fasciatus,

silverfish, firebrat (Thysanura), e.g. Lepisma saccharina and Thermobia domestica,

centipedes (Chilopoda), e.g. Scutigera coleoptrata,

milipedes (Diplopoda), e.g. Narceus spp.,

earwigs (Dermaptera), e.g. forficula auricularia, and

lice (Phthiraptera), e.g. Pediculus humanus capitis, Pediculus humanus corporis, Pthirus pubis, Haematopinus eurysternus, Haematopinus suis, Linognathus vitull, Bovicola bovis, Menopon gallinae, Menacanthus stramineus and Solenopotes capillatus.

The compounds of the formulae I and II, their N-oxides and their salts are also useful for controlling nematodes, especially plant parasitic nematodes such as root knot nematodes, Meloidogyne hapla, Meloidogyne incognita, Meloidogyne javanica, and other Meloidogyne species; cyst-forming nematodes, Globodera rostochiensis and other Globodera species; Heterodera avenae, Heterodera glycines, Heterodera schachtii, Heterodera trifolii, and other Heterodera species; Seed gall nematodes, Anguina species; Stem and foliar nematodes, Aphelenchoides species; Sting nematodes, Belonolaimus longicaudatus and other Belonolaimus species; Pine nematodes, Bursaphelenchus xylophilus and other Bursaphelenchus species; Ring nematodes, Criconema species, Criconemella species, Criconemoides species, Mesocriconema species; Stem and bulb nematodes, Ditylenchus destructor, Ditylenchus dipsaci and other Ditylenchus species; Awl nematodes, Dolichodorus species; Spiral nematodes, Heliocotylenchus multicinctus and other Helicotylenchus species; Sheath and sheathoid nematodes, Hemicycliophora species and Hemicriconemoides species; Hirshmanniella species; Lance nematodes, Hoploaimus species; false rootknot nematodes, Nacobbus species; Needle nematodes, Longidorus elongatus and other Longidorus species; Lesion nematodes, Pratylenchus neglectus, Pratylenchus penetrans, Pratylenchus curvitatus, Pratylenchus goodeyi and other Pratylenchus species; Burrowing nematodes, Radopholus similis and other Radopholus species; Reniform nematodes, Rotylenchus robustus and other Rotylenchus species; Scutellonema species; Stubby root nematodes, Trichodorus primitivus and other Trichodorus species, Paratrichodorus species; Stunt nematodes, Tylenchorhynchus claytoni, Tylenchorhynchus dubius and other Tylenchorhynchus species; Citrus nematodes, Tylenchulus species; Dagger nematodes, Xiphinema species; and other plant parasitic nematode species.

The compounds of the formulae I and II, their N-oxides and their salts are also useful for controlling Arachnoidea, such as arachnids (Acarina), e.g. of the families Argasidae, Ixodidae and Sarcoptidae, such as Amblyomma americanum, Amblyomma variegatum, Ambryomma maculatum, Argas persicus, Boophilus annulatus, Boophilus decoloratus, Boophilus microplus, Dermacentor silvarum, Dermacentor andersoni, Dermacentor variabills, Hyalomma truncatum, Ixodes ricinus, Ixodes rubicundus, Ixodes scapularis, Ixodes holocyclus, Ixodes pacificus, Ornithodorus moubata, Ornithodorus hermsi, Ornithodorus turicata, Ornithonyssus bacoti, Otobius megnini, Dermanyssus gallinae, Psoroptes ovis, Rhipicephalus sanguineus, Rhipicephalus appendiculatus, Rhipicephalus evertsi, Sarcoptes scabiei, and Eriophyidae spp. such as Aculus schlechtendali, Phyllocoptrata oleivora and Eriophyes sheldoni, Tarsonemidae spp. such as Phytonemus pallidus and Polyphagotarsonemus latus; Tenuipalpidae spp. such as Brevipalpus phoenicis; Tetranychidae spp. such as Tetranychus cinnabarinus, Tetranychus kanzawai, Tetranychus pacificus, Tetranychus teiarius and Tetranychus urticae, Panonychus ulmi, Panonychus citri, and Oligonychus pratensis; Araneida, e.g. Latrodectus mactans, and Loxosceles reclusa,

Compounds of the formulae I and II, their pyridine N-oxides and their salts are particularly useful for controlling insects of the order Lepidoptera.

Compounds of the formulae I and II, their pyridine N-oxides and their salts are also particularly useful for controlling insects of the order hemiptera, in particular of the sub-order homoptera.

For use in a method according to the present invention, the compounds I or II, their pyridine N-oxides or their salts can be converted into the customary formulations, e.g. solutions, emulsions, suspensions, dusts, powders, pastes and granules. The use form depends on the particular purpose; it is intended to ensure in each case a fine and uniform distribution of the compound according to the invention.

The formulations are prepared in a known manner, e.g. by extending the active ingredient with solvents and/or carriers, if desired using emulsifiers and dispersants. Solvents/auxiliaries, which are suitable, are essentially:
water, aromatic solvents (for example Solvesso products, xylene), paraffins (for example mineral fractions), alcohols (for example methanol, butanol, pentanol, benzyl alcohol), ketones (for example cyclohexanone, gamma-butyrolactone), pyrrolidones (NMP, NOP), acetates (glycol diacetate), glycols, fatty acid dimethylamides, fatty acids and fatty acid esters. In principle, solvent mixtures may also be used.
carriers such as ground natural minerals (e.g. kaolins, clays, talc, chalk) and ground synthetic minerals (e.g. highly disperse silica, silicates); emulsifiers such as nonionic and anionic emulsifiers (e.g. polyoxyethylene fatty alcohol ethers, alkylsulfonates and arylsulfonates) and dispersants such as lignin-sulfite waste liquors and methylcellulose.

Suitable surfactants are alkali metal, alkaline earth metal and ammonium salts of lignosulfonic acid, naphthalenesulfonic acid, phenolsulfonic acid, dibutylnaphthalenesulfonic acid, alkylarylsulfonates, alkyl sulfates, alkylsulfonates, fatty alcohol sulfates, fatty acids and sulfated fatty alcohol glycol ethers, furthermore condensates of sulfonated naphthalene and naphthalene derivatives with formaldehyde, condensates of naphthalene or of naphthalenesulfonic acid with phenol and formaldehyde, polyoxyethylene octylphenyl ether, ethoxylated isooctylphenol, octylphenol, nonylphenol, alkylphenyl polyglycol ethers, tributylphenyl polyglycol ether, tristearylphenyl polyglycol ether, alkylaryl polyether alcohols, alcohol and fatty alcohol/ethylene oxide condensates, ethoxylated castor oil, polyoxyethylene alkyl ethers, ethoxylated polyoxypropylene, lauryl alcohol polyglycol ether acetal, sorbitol esters, lignin-sulfite waste liquors and methylcellulose.

Substances which are suitable for the preparation of directly sprayable solutions, emulsions, pastes or oil dispersions are mineral oil fractions of medium to high boiling point, such as kerosene or diesel oil, furthermore coal tar oils and oils of vegetable or animal origin, aliphatic, cyclic and aromatic hydrocarbons, for example toluene, xylene, paraffin, tetrahydronaphthalene, alkylated naphthalenes or their derivatives, methanol, ethanol, propanol, butanol, cyclohexanol, cyclohexanone, isophorone, strongly polar solvents, for example dimethyl sulfoxide, N-methylpyrrolidone and water.

Powders, materials for spreading and dusts can be prepared by mixing or concomitantly grinding the active substances with a solid carrier.

Granules, for example coated granules, impregnated granules and homogeneous granules, can be prepared by binding the active ingredients to solid carriers. Examples of solid carriers are mineral earths such as silica gels, silicates, taIc, kaolin, attaclay, limestone, lime, chalk, bole, loess, clay, dolomite, diatomaceous earth, caIcium sulfate, magnesium sulfate, magnesium oxide, ground synthetic materials, fertilizers, such as, for example, ammonium sulfate, ammonium phosphate, ammonium nitrate, ureas, and products of vegetable origin, such as cereal meal, tree bark meal, wood meal and nutshell meal, cellulose powders and other solid carriers.

In general, the formulations comprise from 0.01 to 95% by weight, preferably from 0.1 to 90% by weight, of the active ingredient. The active ingredients are employed in a purity of from 90% to 100%, preferably 95% to 100% (according to NMR spectrum).

The following are examples of formulations: 1. Products for dilution with water

### A Soluble Concentrates (SL)

10 parts by weight of a compound according to the invention are dissolved in water or in a water-soluble solvent. As an alternative, wetters or other auxiliaries are added. The active ingredient dissolves upon dilution with water.

### B Dispersible Concentrates (DC)

20 parts by weight of a compound according to the invention are dissolved in cyclohexanone with addition of a dispersant, for example polyvinylpyrrolidone. Dilution with water gives a dispersion.

### C Emulsifiable Concentrates (EC)

15 parts by weight of a compound according to the invention are dissolved in xylene with addition of calcium dodecylbenzenesulfonate and castor oil ethoxylate (in each case 5% strength). Dilution with water gives an emulsion.

### D Emulsions (EW, EO)

40 parts by weight of a compound according to the invention are dissolved in xylene with addition of calcium dodecylbenzenesulfonate and castor oil ethoxylate (in each case 5% strength). This mixture is introduced into water by means of an emulsifier (Ultraturrax) and made into a homogeneous emulsion. Dilution with water gives an emulsion.

### E Suspensions (SC, OD)

In an agitated ball mill, 20 parts by weight of a compound according to the invention are milled with addition of dispersant, wetters and water or an organic solvent to give a fine active ingredient suspension. Dilution with water gives a stable suspension of the active ingredient.

### F Water-Dispersible Granules and Water-Soluble Granules (WG, SG)

50 parts by weight of a compound according to the invention are ground finely with addition of dispersants and wetters and made into water-dispersible or water-soluble granules by means of technical appliances (for example extrusion, spray tower, fluidized bed). Dilution with water gives a stable dispersion or solution of the active ingredient.

### G Water-Dispersible Powders and Water-Soluble Powders (WP, SP)

75 parts by weight of a compound according to the invention are ground in a rotor-stator mill with addition of dispersant, wetters and silica gel. Dilution with water gives a stable dispersion or solution with the active ingredient.

### 2. Products to be Applied Undiluted

### H Dustable Powders (DP)

5 parts by weight of a compound according to the invention are ground finely and mixed intimately with 95% of finely divided kaolin. This gives a dustable product.

### I Granules (GR, FG, GG, MG)

0.5 parts by weight of a compound according to the invention is ground finely and associated with 95.5% carriers. Current methods are extrusion, spray drying or the fluidized bed. This gives granules to be applied undiluted.

### J ULV Solutions (UL)

10 parts by weight of a compound according to the invention are dissolved in an organic solvent, for example xylene. This gives a product to be applied undiluted.

The active ingredients can be used as such, in the form of their formulations or the use forms prepared therefrom, eg. in the form of directly sprayable solutions, powders, suspensions or dispersions, emulsions, oil dispersions, pastes, dustable products, materials for spreading, or granules, by means of spraying, atomizing, dusting, spreading or pouring. The use forms depend entirely on the intended purposes; it is intended to ensure in each case the finest possible distribution of the active ingredients according to the invention.

Aqueous use forms can be prepared from emulsion concentrates, pastes or wettable powders (sprayable powders, oil dispersions) by adding water. To prepare emulsions, pastes or oil dispersions, the substances, as such or dissolved in an oil or solvent, can be homogenized in water by means of a wetter, tackifier, dispersant or emulsifier. Alternatively, it is possible to prepare concentrates composed of active substance, wetter, tackifier, dispersant or emulsifier and, if appropriate, solvent or oil, and such concentrates are suitable for dilution with water.

The active ingredient concentrations in the ready-to-use products can be varied within relatively wide ranges. In general, they are from 0.0001 to 10%, preferably from 0.01 to 1%.

The active ingredients may also be used successfully in the ultra-low-volume process (ULV), it being possible to apply formulations comprising over 95% by weight of active ingredient, or even to apply the active ingredient without additives.

Compositions which can be used according to the invention may also contain other active ingredients, for example other pesticides such as insecticides and herbicides, fertilizers such as ammonium nitrate, urea, potash, and superphosphate, phytotoxicants and plant growth regulators, safeners and nematicides. These additional ingredients may be used sequentially or in combination with the above-described compositions, if appropriate also added only immediately prior to use (tank mix). For example, the plant(s) may be sprayed with a composition of this invention either before or after being treated with other active ingredients.

These agents usually are admixed with the agents according to the invention in a weight ratio of 1:100 to 100:1.

The following list of pesticides together with which the compounds I and II according to the invention can be used, is intended to illustrate the possible combinations, but not to impose any limitation:

Organo(thio)phosphates: e.g. acephate, azamethiphos, azinphos-methyl, chlorpyrifos, chlorpyrifos-methyl, chlorfenvinphos, diazinon, dichlorvos, dicrotophos, dimethoate, disulfoton, ethion, fenitrothion, fenthion, isoxathion, malathion, methamidophos, methidathion, methyl-parathion, mevinphos, monocrotophos, oxydemeton-methyl, paraoxon, parathion, phenthoate, phosalone, phosmet, phosphamidon, phorate, phoxim, pirimiphos-methyl, profenofos, prothiofos, sulprophos, tetrachlorvinphos, terbufos, triazophos, trichlorfon;

Carbamates: e.g. alanycarb, aldicarb, bendiocarb, benfuracarb, carbaryl, carbofuran, carbosulfan, fenoxycarb, furathiocarb, methiocarb, methomyl, oxamyl, pirimicarb, propoxur, thiodicarb, triazamate;

Pyrethroids: allethrin, bifenthrin, cyfluthrin, cyhalothrin, cyphenothrin, cypermethrin, alpha-cypermethrin, beta-cypermethrin, zeta-cypermethrin, deltamethrin, esfenvalerate, etofenprox, fenpropathrin, fenvalerate, imiprothrin, lambda-cyhalothrin, permethrin, prallethrin, pyrethrin I and II, resmethrin, silafluofen, tau-fluvalinate, tefluthrin, tetramethrin, tralomethrin, transfluthrin, profluthrin, dimefluthrin;

Arthropod growth regulators: a) chitin synthesis inhibitors: e.g. benzoylureas: chlorfluazuron, diflubenzuron, flucycloxuron, flufenoxuron, hexaflumuron, lufenuron, novaluron, teflubenzuron, triflumuron; buprofezin, diofenolan, hexythiazox, etoxazole, clofentazine; b) ecdysone antagonists: e.g. halofenozide, methoxyfenozide, tebufenozide, azadirachtin; c) juvenoids: e.g. pyriproxyfen, methoprene, fenoxycarb; d) lipid biosynthesis inhibitors: e.g. spirodiclofen, spiromesifen or spirotetramat;

Nicotinic receptor agonists/antagonists compounds (nicotinoid insecticides or neonicotinoids): e.g. clothianidin, dinotefuran, imidacloprid, thiamethoxam, nitenpyram, acetamiprid, thiacloprid or the thiazol compound of formula P1

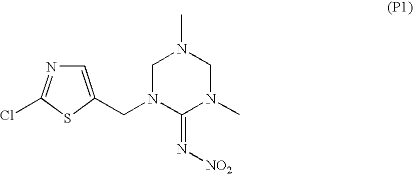

GABA antagonist compounds: e.g. acetoprole, endosulfan, ethiprole, fipronil, vaniliprole, pyrafluprole, pyriprole, or the phenylpyrazole compound of formula P2 (5-amino-3-(aminothiocarbonyl)-1-(2,6-dichloro-4-trifluoromethylphenyl)-4-(trifluoromethylsulfinyl)-pyrazole)

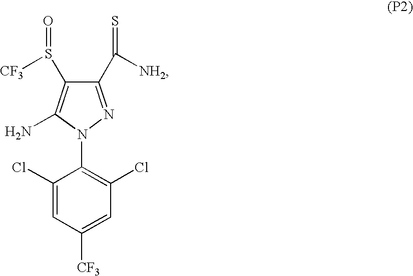

Macrocyclic lactone insecticides: abamectin, emamectin, milbemectin, lepimectin, spinosad,

Mitochondrial complex I electron transport inhibitors (METI I compounds): e.g. fenazaquin, pyridaben, tebufenpyrad, tolfenpyrad, flufenerim;

Mitochondrial complex II and/or complex III electron transport inhibitors (METI II and III compounds): e.g. acequinocyl, fluacyprim, hydramethylnon;

various: amitraz, azadirachtin, bifenazate, cartap, chlorfenapyr, chlordimeform, cyromazine, diafenthiuron, diofenolan, formetanate, formetanate-hydrochlorid, indoxacarb, metaflumizon, piperonylbutoxid, pymetrozine, thiocyclam, pyridalyl, flonicamid, flupyrazofos, NC 512, flubendiamide, bistrifluoron, benclothiaz amidoflumet, cyflumetofen, profluthrin, dimefluthrin, amidrazone, N-Ethyl-2,2-dichlor-1-methyIcyclopropanecarboxamid-2-(2,6-dichlor-α.α.α-tri-fluoro-p-tolyl)hydrazon, N-ethyl-2,2-dimethylpropionamide-2-(2,6-dichloro-α.α.α-trifluoro-p-tolyl)-hydrazon metaflumizone, and the pesticide of the following formula as described in WO 98/05638:

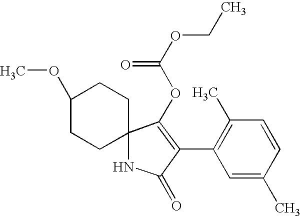

Aminoisothiazoles of the formula

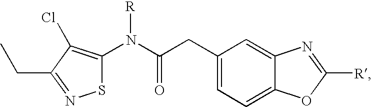

wherein

### R=—CH₂O CH₃ or H and

### R=—CF₂CF₂ CF₃;

Anthranilamides of the formula

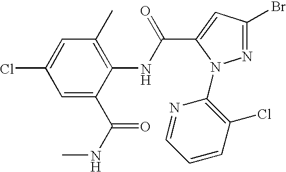

and compounds of the formulae

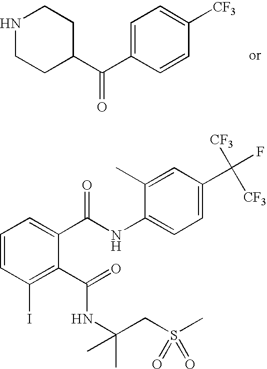

The aforementioned compositions are particularly useful for protecting plants against infestation of said pests or to combat these pests in infested plants.

However, the compounds of formulae I and II, their pyridine N-oxides and their salts are also suitable for the treatment of seeds. Application to the seeds is carried out before sowing, either directly on the seeds or after having pregerminated the latter.

Compositions which are useful for seed treatment are e.g.:

A Soluble concentrates (SL, LS)

### D Emulsions (EW, EO, ES)

### E Suspensions (SC, OD, FS)

F Water-dispersible granules and water-soluble granules (WG, SG)
G Water-dispersible powders and water-soluble powders (WP, SP, WS)
H Dustable powders (DP, DS)

Preferred FS formulations of compounds of formulae I or II for seed treatment usually comprise from 0.5 to 80% of the active ingredient, from 0.05 to 5% of a wetter, from 0.5 to 15% of a dispersing agent, from 0.1 to 5% of a thickener, from 5 to 20% of an anti-freeze agent, from 0.1 to 2% of an anti-foam agent, from 1 to 20% of a pigment and/or a dye, from 0 to 15% of a sticker/adhesion agent, from 0 to 75% of a filler/vehicle, and from 0.01 to 1% of a preservative.

Suitable pigments or dyes for seed treatment formulations are pigment blue 15:4, pigment blue 15:3, pigment blue 15:2, pigment blue 15:1, pigment blue 80, pigment yellow 1, pigment yellow 13, pigment red 112, pigment red 48:2, pigment red 48:1, pigment red 57:1, pigment red 53:1, pigment orange 43, pigment orange 34, pigment orange 5, pigment green 36, pigment green 7, pigment white 6, pigment brown 25, basic violet 10, basic violet 49, acid red 51, acid red 52, acid red 14, acid blue 9, acid yellow 23, basic red 10, basic red 108.

Stickers/adhesion agents are added to improve the adhesion of the active materials on the seeds after treatment. Suitable adhesives are block copolymers EO/PO surfactants but also polyvinylalcohols, polyvinylpyrrolidones, polyacrylates, polymethacrylates, polybutenes, polyisobutylenes, polystyrene, polyethyleneamines, polyethyleneamides, polyethyleneimines (Lupasol®, Polymin®), polyethers and copolymers derived from these polymers.

For use against ants, termites, wasps, flies, mosquitoes, crickets, or cockroaches, compounds of formula I or II, their pyridine N-oxides or their salts are preferably used in a bait composition.

The bait can be a liquid, a solid or a semisolid preparation (e.g. a gel). Solid baits can be formed into various shapes and forms suitable to the respective application e.g. granules, blocks, sticks, disks. Liquid baits can be filled into various devices to ensure proper application, e.g. open containers, spray devices, droplet sources, or evaporation sources. Gels can be based on aqueous or oily matrices and can be formulated to particular necessities in terms of stickyness, moisture retention or aging characteristics.

The bait employed in the composition is a product which is sufficiently attractive to incite insects such as ants, termites, wasps, flies, mosquitoes, crickets etc. or cockroaches to eat it. The attractiveness can be manipulated by using feeding stimulants or sex pheromones. Food stimulants are chosen, for example, but not exclusively, from animal and/or plant proteins (meat-, fish- or blood meal, insect parts, egg yolk), from fats and oils of animal and/or plant origin, or mono-, oligo- or polyorganosaccharides, especially from sucrose, lactose, fructose, dextrose, glucose, starch, pectin or even molasses or honey. Fresh or decaying parts of fruits, crops, plants, animals, insects or specific parts thereof can also serve as a feeding stimulant. Sex pheromones are known to be more insect specific. Specific pheromones are described in the literature and are known to those skilled in the art.

Formulations of compounds of formulae I or II as aerosols (e.g in spray cans), oil sprays or pump sprays are highly suitable for the non-professional user for controlling pests such as flies, fleas, ticks, mosquitoes or cockroaches. Aerosol recipes are preferably composed of the active compound, solvents such as lower alcohols (e.g. methanol, ethanol, propanol, butanol), ketones (e.g. acetone, methyl ethyl ketone), paraffin hydrocarbons (e.g. kerosenes) having boiling ranges of approximately 50 to 250° C., dimethylformamide, N-methylpyrrolidone, dimethyl sulfoxide, aromatic hydrocarbons such as toluene, xylene, water, furthermore auxiliaries such as emulsifiers such as sorbitol monooleate, oleyl ethoxylate having 3-7 mol of ethylene oxide, fatty alcohol ethoxylate, perfume oils such as ethereal oils, esters of medium fatty acids with lower alcohols, aromatic carbonyl compounds, if appropriate stabilizers such as sodium benzoate, amphoteric surfactants, lower epoxides, triethyl orthoformate and, if required, propellants such as propane, butane, nitrogen, compressed air, dimethyl ether, carbon dioxide, nitrous oxide, or mixtures of these gases.

The oil spray formulations differ from the aerosol recipes in that no propellants are used.

The compounds of formulae I or II, their pyridine N-oxides, their salts and their respective compositions can also be used in mosquito and fumigating coils, smoke cartridges, vaporizer plates or long-term vaporizers and also in moth papers, moth pads or other heat-independent vaporizer systems.

The compounds of formulae I or II, their pyridine N-oxides, their salts, and their compositions can be used for protecting non-living material, in particular cellulose-based materials such as wooden materials e.g. trees, board fences, sleepers, etc. and buildings such as houses, outhouses, factories, but also construction materials, furniture, leathers, fibers, vinyl articles, electric wires and cables etc. from ants and/or termites, and for controlling ants and termites from doing harm to crops or human being (e.g. when the pests invade into houses and public facilities). The compounds of formula I are applied not only to the surrounding soil surface or into the under-floor soil in order to protect wooden materials but it can also be applied to lumbered articles such as surfaces of the under-floor concrete, alcove posts, beams, plywoods, furniture, etc., wooden articles such as particle boards, half boards, etc. and vinyl articles such as coated electric wires, vinyl sheets, heat insulating material such as styrene foams, etc. In case of application against ants doing harm to crops or human beings, the ant controller of the present invention is applied to the crops or the surrounding soil, or is directly applied to the nest of ants or the like.

In the methods according to the invention the pests are controlled by contacting the target parasite/pest, its food supply, habitat, breeding ground or its locus with a pesticidally effective amount of compounds of formulae I or II or with an pyridine N-oxide thereof or with a salt thereof or with a composition, containing a pesticidally effective amount of a compound of formula I or II, or a pyridine N-oxide or a salt thereof.

“Locus” means a habitat, breeding ground, plant, seed, soil, area, material or environment in which a pest or parasite is growing or may grow.

In general, “pesticidally effective amount” means the amount of active ingredient needed to achieve an observable effect on growth, including the effects of necrosis, death, retardation, prevention, and removal, destruction, or otherwise diminishing the occurrence and activity of the target organism. The pesticidally effective amount can vary for the various compounds/compositions used in the invention. A pesticidally effective amount of the compositions will also vary according to the prevailing conditions such as desired pesticidal effect and duration, weather, target species, locus, mode of application, and the like.

The compounds of the invention can also be applied preventively to places at which occurrence of the pests is expected.

The compounds of formulae I or II, their pyridine N-oxides and their salts may be also used to protect growing plants from attack or infestation by pests by contacting the plant with a pesticidally effective amount of compounds of formula I or II, their pyridine N-oxides and their salts. As such, “contacting” includes both direct contact (applying the compounds/compositions directly on the pest and/or plant—typically to the foliage, stem or roots of the plant) and indirect contact (applying the compounds/compositions to the locus of the pest and/or plant).

For use in treating crop plants, the rate of application of the active ingredients of this invention may be in the range of 0.1 g to 4000 g per hectare, desirably from 25 g to 600 g per hectare, more desirably from 50 g to 500 g per hectare.

In the treatment of seed, the application rates of the mixture are generally from 0.1 g to 10 kg per 100 kg of seed, preferably from 1 g to 5 kg per 100 kg of seed, in particular from 1 g to 200 g per 100 kg of seed.

In the case of soil treatment or of application to the pests dwelling place or nest, the quantity of active ingredient ranges from 0.0001 to 500 g per 100 m², preferably from 0.001 to 20 g per 100 m².

Customary application rates in the protection of materials are, for example, from 0.01 g to 1000 g of active compound per m² treated material, desirably from 0.1 g to 50 g per m².

Insecticidal compositions for use in the impregnation of materials typically contain from 0.001 to 95 weight %, preferably from 0.1 to 45 weight %, and more preferably from 1 to 25 weight % of at least one repellent and/or insecticide.

For use in bait compositions, the typical content of active ingredient is from 0.001 weight % to 15 weight %, desirably from 0.001 weight % to 5% weight % of active compound.

For use in spray compositions, the content of active ingredient is from 0.001 to 80 weights %, preferably from 0.01 to 50 weight % and most preferably from 0.01 to 15 weight %.

The present invention is now illustrated in further detail by the following examples.

The products were characterized by coupled High Performance Liquid Chromatography/mass spectrometry (HPLC/MS), by ¹H-NMR or by their melting point.

For HPLC An analytical RP-18 column (Chromolith Speed ROD, dimensions 50×4.6 mm, from Merck KGaA, Germany), which was operated at 30 and 40° C., at a flow-rate of 1.8 ml/min and an injection volume of 2 μl was used. The mobile phase used was acetonitrile/water with 0.1% trifluoroacetic acid in a ratio of 5:95 to 95:5 in 5 minutes). HPLC was carried out using either method A (high speed overview method; recorded on Agilent 1100 LC/MSD) or method B (slow method; recorded on Agilent 1100 LC/MSD). Mass spectrometry was carried out using a quadrupole mass spectrometer with electrospray ionization at 80V in the positive mode.

In the examples the following abbreviations are used:

m.p. melting point
eq. equivalent(s)

### THF Tetrahydrofurane

### CH Cyclohexane

EE Ethyl acetate
MS mass spectrum
m multiplett
s singulett
t triplett
q quartett

### I. PREPARATION EXAMPLES

The reactions described below can be run either with enantiomerical pure R or S compounds or racemates yielding either the enantiomeric pure (enriched) or racemic products.

### Example 1

### 2-(5-chloropyridin-3-yloxymethyl)-pyrrolidin-1-yl benzoic acid ester

A mixture of 10.23 g (48.1 mmol) of R-3-Chloro-5-(pyrrolidin-2-ylmethyloxy)-pyridine, 19.1 g of benzoyl peroxide (59 mmol) and 55 g poly-(4-ethenylpyridin (480 mmol) were agitated in 300 ml THF at ambient temperature for 24 h. The polymer was filtered off, rinsed with 100 ml THF and the solvent of the combined organic solutions was removed in vacuo. The residue was subjected to column chromatographie (CH/EE 9/1 to 7/1) to afford 11.1 g of the title compound.

### Example 2

### 2-(5-chloropyridin-3-yloxymethyl)-pyrrolidin-1-ol

11.0 g (0.0334 mol) of the compound of example 1 were dissolved in 200 ml of methanol and 2.53 g (0.047 mol) NaOCH₃ were added and the mixture was stirred for 1 h. After evaporation of the solvent and column chromatographie of the residue (CH/EE 1/1->EE) 7.0 g of the title compound were obtained.

### Example 3

### 2,2-Dimethyl-propionic acid 2-(5-chloropyridin-3-yloxymethyl)pyrrolidin-1-yl ester

A mixture of 0.300 g (1.3 mmol) of the compound of examples 2, 0.19 g (1.6 mmol) of 2,2-dimethylpropionicacid cloride and 0.216 g (1.5 mmol) of K₂CO₃ was stirred in 50 ml dichloromethane for 8 h. Filtration of the reaction mixture and chromatography of the residue (CH/EE 3/1) yields 258 mg of the title compound.

### Example 4

### 2-(5-chloropyridin-3-yloxymethyl)-azetidin-1-yl benzoic acid ester

A mixture of 0.36 g (1.8 mmol) 2-(5-chloropyridin-3-yloxymethyl)-azetidin-1-yl, 2.5 g of polyethenylpyridin and 0.54 g (2.2 mmol) of dibenzoylperoxide in 15 ml THF was reacted as described for example 1 to yield 0.065 g of the title compound.

### Example 5

### 2-(5-chloropyridin-3-yloxymethyl)-pyrrolidin-1-carboxamide

A solution of 0.3 g (0.9 mmol) of 2-(5-chloropyridin-3-yloxymethyl)-pyrrolidin-1-ol in 15 ml of THF/water (1:1 v/v) were subsequently treated with 0.1 g concentrated aqueous hydrochloric acid and 0.77 g (9 mmol) of Kaliumcyanat at 0° C. for 1 h. The reaction mixture was evaporated to dryness, the residue was dissolved in ethyl acetate and the organic layer was washed with 5 ml of water. After evaporation of the solvent 170 mg of the title compound were obtained (m.p. 95° C.).

Using essentially the same procedure as described in examples 1 to 5, the compounds shown in table I were prepared and identified by ¹H-NMR or MS spectral analysis or melting point.

In table 1, the letter given in column C* gives the configuration at the indicated carbon atom.

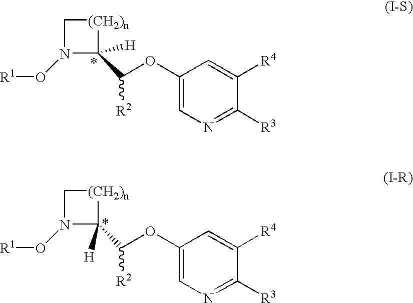

TABLE I
Compounds I

| Exam- | | | | | | | | |
|---|---|---|---|---|---|---|---|---|
| ple | R¹ | R² | R³ | R⁴ | n | C* | mp [° C.] | ¹H-NMR (ppm)-MS (m/z) |
| 1 | —C(O)—C₆H₅ | H | H | Cl | 2 | S | | (DMSO d₆): 8.2 (m, 1 H), 8.1 (m, 1 H), 7.8 (m, 2 H), 7.5 (m, 4 H), |
| | | | | | | | | 4.3 (m, 2 H), 3.7 (m, 1 H), 3.5 (m, 1 H), 3.1 (m, 1 H), |
| | | | | | | | | 2.1 m, 1 H), 2.0-1.6 (m, 3 H). |
| 2 | H | H | H | Cl | 2 | S | | (DMSO d₆): 8.3 (m, 1 H), 8.2 (m, 1 H), 8.0 (m, 1 H), 7.6 (s, 1 H), |
| | | | | | | | | 4.2-4.0 (m, 2 H), 3.1-3.0 (m, 2 H), 2.7 (m, 1 H), |
| | | | | | | | | 2.0-1.5 (m, 4 H). |
| 3 | —C(O)—C(CH₃)₃ | H | H | Cl | 2 | S | | (CDCl₃): 8.2 (m, 2 H), 7.2 (m, 1 H), 4.2 (m, 1 H), 4.0 (m 1 H), |
| | | | | | | | | 3.5 (m, 2 H), 3.0 (m, 1 H), 2.2 (m 1 H), 2.0 (m, 2 H), 1.8 (m, 1 H), |
| | | | | | | | | 1.2 (s, 9 H), |
| 4 | C(O)—C₆H₅ | H | H | Cl | 1 | S | | 8.2 (m, 2 H), 8.0 (m, 1 H), 7.6 (m, 1 H), 7.5 (m, 2 H), 7.2 (m, 1 H), |
| | | | | | | | | 4.3 (m 3 H), 4.1 (m, 1 H), 3.7 (m, 1 H), 2.4 (m, 1 H), 2.2 (m, 1 H). |
| 5 | —C(O)—NH₂ | H | H | Cl | 2 | S | 95 | |
| 6 | —C(O)—CH(CH₃)₂ | H | Cl | H | 2 | S | | (CDCl₃): 8.0 (m, 1 H), 7.2 (m, 2 H), 4.2 (m, 1 H), |
| | | | | | | | | 4.0 (m, 1 H), 3.5 (m, 2 H), 3.0 (m, 1 H), 2.6 (sept. 1 H), 2.2-2.0 (m, |
| | | | | | | | | 3 H), 1.8 (m, 1 H), 1.2 (d, 6 H). |
| 7 | —C(O)—C₆H₅ | H | Cl | H | 2 | S | | (CDCl₃): 8.0 (m, 3 H), 7.6 m, 1 H), 7.5 (m, 2 H), 7.1 (m, 2 H) 4.3 (m, |
| | | | | | | | | 1 H), 4.1 (m, 1 H), 3.7 (m, 2 H), 3.2 (m, 1 H), 2.2 (m. 1 H), |
| | | | | | | | | 2.1-2.0 (m, 2 H), 1.8 (m, 1 H), |
| 8 | H | H | Cl | H | 2 | S | | (CDCl₃): 8.1 (m, 1 H), 7.3-7.1 (m, 2 H), 4.3 (s, 1 H), 4.1 (m, 1 H) |
| | | | | | | | | 3.3 (m, 1 H), 3.2 (m, 1 H), 3.0 (m, 1 H), 2.1-1.2 (m, 4 H) |
| 9 | —C(O)—CH₂CH₃ | H | Cl | H | 2 | S | | (CDCl₃): 8.1 (m, 1 H), 7.3 (m, 2 H), 4.2-4.0 (m, 2 H), 3.5 (m, 2 H), |
| | | | | | | | | 3.0 (m, 1 H), 2.3 (q 2 H), 2.2-1.9 (m, 3 H), 1.7 (m, 1 H), 1.2 (t, 3 H), |
| 10 | —C(O)—CH₃ | H | Cl | H | 2 | S | | (CDCl₃): 8.0 (m, 1 H), 7.3 (m, 2 H), 4.2-4.0 (m, 2 H), 3.5 (m, 2 H), |
| | | | | | | | | 3.0 (m 1 H), 2.2 (m, 1 H), 2.0 (s, 3 H), 1.9 (m, 2 H), 1.7 (m, 1 H), |
| 11 | —C(O)—CH(CH₃)₂ | H | H | Cl | 2 | S | | (CDCl₃): 8.1 (m, 2 H), 7.2 (m, 1 H), 4.2-4.0 (m, 2 H), 3.5 (m, 2 H), |
| | | | | | | | | 3.0 (m 1 H), 2.5 (sept 1 H), 2.2 m, 1 H); 2.0 m 2 H), |
| | | | | | | | | 1.7 (m1 H), 1.2 (d, 6H). |
| 12 | —C(O)—CH₂CH(CH₃)₂ | H | H | Cl | 2 | S | | (CDCl₃): 8.1 (m, 2 H), 7.3 (m 1 H), 4.2-4.0 (m, 2 H), |
| | | | | | | | | 3.5 (m, 2 H), 3.0 (m 1 H), 2.2-1.7 (6 H), 1.0 (dt 6 H) |
| 13 | —C(O)—CH₃ | H | H | Cl | 2 | S | | (CDCl₃): 8.2 (m, 2 H), 7.3 (m, 1 H), 4.2-4.0 (m, 2 H), 3.5 (m 2 H), |
| | | | | | | | | 3.0 (m, 1 H), 2.2 (m, 1 H), 2.1 (s, 3 H), 2.0 (m, 2 H), 1.8 (M, 1 H) |
| 14 | —C(O)—C(CH₃)₃ | H | Cl | H | 2 | S | | (CDCl₃): 8.0 (m, 1 H), 7.2 (m, 2 H), 4.2-4.0 (m, 2 H), |
| | | | | | | | | 3.5 (m, 2 H), 3.0 (m, 1 H), 2.2-1.8 (m, 4 H), 1.2 (s, 9 H), |
| 15 | —C(O)—CH₂CH(CH₃)₂ | H | Cl | H | 2 | R | | (CDCl₃): 8.0 (m, 1 H), 7.2 (m, 2 H), 4.2-4.0 (m, 2 H), |
| | | | | | | | | 3.5 (m, 2 H), 3.0 (m, 1 H), 2.2-1.6 (m, 6 H), 1.0 (d, 6 H) |
| 16 | —C(O)—C₆H₅ | H | Cl | Cl | 2 | S | | (CDCl₃): 8.0 (m, 2 H), 7.8 (m, 1 H), 7.6 (m, 1 H), 7.5 (m, 2 H), 7.3 |
| | | | | | | | | (m, 1 H), 4.3-4.0 (m, 2 H), 3.7 (m, 2 H), 3.2 (m, 1 H), 2.3-1.8 (m 4H) |
| 17 | —C(O)—C₆H₅ | H | Cl | F | 2 | S | | (CDCl₃): 8.0 (m, 2 H), 7.7 (m, 1 H), 7.6 (m, 1 H), 7.5 (m, 2 H), 7.0 |
| | | | | | | | | (m, 1 H), 4.3-4.1 (m 2 H), 3.7 (m, 2 H), 3.3 (m, 1 H), |
| | | | | | | | | 2.3-1.8 (m, 4 H) |
| 18 | —C(O)—CH₃ | H | Cl | F | 2 | S | 91-93 | (CDCl₃): 8.0 (m, 1 H), 7.0 (m, 1 H), 4.2-4.0 (m, 2 H), 3.5 (m, 2 H), |
| | | | | | | | | 3.0 (m, 1 H); 2.2 (m, 1 H), 2.1 (s, 3 H), 2.0-1.7 (m, 3 H) |
| 19 | H | H | Cl | Cl | 2 | R | | (CDCl₃): 8.0 (m, 1 H), 7.3 (m, 1 H), 5.2 (br 1 H), 4.2 (m, 2 H), |
| | | | | | | | | 3.3-3.2 (m, 2 H), 2.9 (m, 1 H), 2.2-1.6 (m, 4 H) |
| 20 | —C(O)—CH₃ | H | Cl | Cl | 2 | S | | (CDCl₃): 8.0 (m, 1 H), 7.4 (m 1 H), 4.2-4.0 (m, 2 H), 3.5 (m, 2 H), |
| | | | | | | | | 3.0 (m, 1 H), 2.2 (m, 1 H), 2.0 (s, 3 H), 1.8 (m, 2 H), 1.7 (m, 1 H) |
| 21 | —C(O)—C(CH₃)₃ | H | Cl | Cl | 2 | R | 79 | (CDCl₃): 8.0 (m, 1 H), 7.3 (m, 1 H), 4.2-4.0 (m, 2 H), |
| | | | | | | | | 3.5 (m, 2 H), 3.0 (m, 1 H), 2.2 (m, 1 H), 2.0 (m, 2 H), 1.7 (m, 1 H), |
| | | | | | | | | 1.2 (s, 9 H) |
| 22 | —C(O)—CH₂CH₃ | H | Cl | Cl | 2 | S | 73-74 | MS (ESI): [M + H]⁺ 318 |
| 23 | —C(O)—-CH(CH₃)₂ | H | Cl | Cl | 2 | S | 92-93 | (CDCl₃): 8.0 (m 1 H), 7.3 (m, 1 H), 4.2-4.0 (m, 2 H), 3.5 (m, 2 H), |
| | | | | | | | | 3.0 (m, 1 H), 2.5 (sept., 1 H), 2.2 (m, 1 H), 1.9 (m, 2 H), 1.7 (m 1 |
| | | | | | | | | H), 1.3 (d, 6 H) |
| 24 | —C(O)—CH₂CH(CH₃)₂ | H | Cl | Cl | 2 | S | | (CDCl₃): 8.0 (m, 1 H), 7.3 (m, 21 H), 4.2-4.0 (m, 2 H), 3.5 (m, 2 H), |
| | | | | | | | | 3.0 (m, 1 H), 2.2-1.9 (m, 6 H), 1.7 (m, 1 H), 0.9 (d, 6 H) |
| 25 | —C(O)—C(CH₃)₃ | H | Cl | Cl | 2 | S | 84-85 | (CDCl₃): 8.0 (m, 1 H), 7.3 (m, 1 H), 4.2-4.0 (m 2 H), 3.5 (m, 2 H), |
| | | | | | | | | 3.0 (m, 1 H), 2.2-1.9 (m, 3 H), 1.7 (m, 1 H), 1.2 (s, 9 H) |
| 26 | —C(O)-(4-Cl—C₆H₄) | H | Cl | Cl | 2 | S | | (CDCl₃): 8.2-8.1 (m, 2 H), 7.9 (m, 2 H), 7.4 (m, 2 H), 7.1 (m, 1 H), |
| | | | | | | | | 4.2-4.1 (m, 2 H), 3.7 (m, 2 H), 3.2 (m, 1 H), 2.2 (m, 1 H), 2.0 (m, 2 |
| | | | | | | | | H), 1.8 (m, 1 H) |
| 27 | —C(O)-[4-(OCH₃)—C₆H₄] | H | Cl | Cl | 2 | S | | (CDCl₃): 8.1 (m, 1 H), 7.9 (m, 2 H), 7.3 (m, 1 H), |
| | | | | | | | | 7.1 (m, 1 H), 6.9 (m, 2 H), 4.3-4.1 (m, 2 H), 3.8 (s, 3 H), |
| | | | | | | | | 3.7 (m, 2 H), 3.2 (m, 1 H) |
| 28 | H | H | Cl | Cl | 2 | S | 111-112 | |
| 29 | —C(O)—CH₂—OCH₃ | H | Cl | Cl | 2 | S | | (CDCl₃): 8.0 (m, 1 H), 7.3 (m, 1 H), 4.2-4.0 (m, 2 H), 4.1 s, 2 H), |
| | | | | | | | | 3.6 (m, 2 H), 3.4 (s, 3 H), 3.1 (m, 1 H), 2.2-1.8 (m, 4 H). |
| 30 | —C(O)—OCH₃ | H | Cl | Cl | 2 | S | | (CDCl₃): 8.0 (m, 1 H), 7.3 (m, 1 H), 4.2-4.1 (m, 2 H), |
| | | | | | | | | 3.8 (m, 1 H), 3.6 (m, 2 H), 3.1 (m, 1 H), 2.2-1.8 (m, 4 H) |
| 31 | —C(O)—OCH₃ | H | H | Cl | 2 | S | | (CDCl₃): 8.2 (m, 2 H), 7.2 (m, 1 H), 4.2 (m, 1 H), 4.0 (m 1 H), |
| | | | | | | | | 3.6 (m, 2 H), 3.4 (s, 3 H). 3.0 (m, 1 H), 2.2 (m 1 H), 2.0 (m, 2 H). |
| 32 | —C(O)-(2-thienyl) | H | H | Cl | 2 | S | | (CDCl₃): 8.2 (m, 1 H), 8.1 (m, 1 H), 7.8 (m, 1 H), 7.6 (m, 1 H), |
| | | | | | | | | 7.1 (m, 2 H), 4.3-4.1 (m, 2 H), 3.7 (m, 2 H), 3.2 (m, 1 H), |
| | | | | | | | | 2.2-1.8 (m, 4 H) |
| 33 | —C(O)—C(CH3)₂CH₂CH₃ | H | H | Cl | 2 | S | | (CDCl₃): 8.0 (m 2 H), 7.2 (m, 2 H), 4.2-4.0 (m, 2 H), 3.5 (m, 2 H), |
| | | | | | | | | 3.0 (m, 1 H), 2.2 (m, 1 H), 2.0 (m, 2 H), 1.8 (m, 1 H), 1.6 (q 2 h), 1.2 |
| | | | | | | | | (s, 6 H), 0.8 (t 3 H) |
| 34 | —C(O)—C(CH₃)₂CH₂CH₃ | H | Cl | H | 2 | S | | (CDCl₃): 8.0 (m 1 H), 7.2 (m, 2 H), 4.2-4.0 (m, 2 H), 3.5 (m, 2 H), |
| | | | | | | | | 3.0 (m, 1 H), 2.2 (m, 1 H), 2.0 (m, 2 H), 1.8 (m, 1 H), 1.6 (q 2 h), 1.2 |
| | | | | | | | | (s, 6 H), 0.8 (t 3 H) |
| 35 | —C(O)—C(CH₃)₂CH₂CH₃ | H | Cl | Cl | 2 | S | | (CDCl₃): 8.0 (m 1 H), 7.2 (m, 1 H), 4.2-4.0 (m, 2 H), 3.5 (m, 2 H), |
| | | | | | | | | 3.0 (m, 1 H), 2.2 (m, 1 H), 2.0 (m, 2 H), 1.8 (m, 1 H), 1.6 (q 2 h), 1.2 |
| | | | | | | | | (s, 6 H), 0.8 (t 3 H) |
| 36 | —C(O)—NH₂ | H | Cl | Cl | 2 | S | 114 | |
| 37 | —C(O)—CH(CH₃)₂ | H | Cl | F | 2 | S | | MS-ESI: [M + H]⁺ 317 |
| 38 | C(O)—C₆H₅ | H | H | F | 2 | S | | (CDCl₃): 8.1 (m, 2 H), 8.0 (m, 2 H), 7.6 (m 1 H), 7.4 (m, 2 H), |
| | | | | | | | | 6.7 (m, 1 H), 4.3 (m, 1 H), 4.1 (m, 1 H), 3.7 (m, 2 H), 3.2 (m, 1 H), |
| | | | | | | | | 2.3 (m, 1 H), 2.0 (m, 2 H), 1.6 (m, 1 H) |
| 39 | —C(O)—C(CH₃)₂CH₂CH₃ | H | H | F | 2 | S | | (CDCl₃): 8.1 (m, 2 H), 7.0 (m, 1 H), 4.2 (m, 1 H), 4.0 (m, 1 H), |
| | | | | | | | | 3.5 (m, 2 H), 3.0 (m, 1 H), 2.2 (m, 1 H), 2.0 (m, 2 H), |
| | | | | | | | | 1.8 (m, 1 H), 1.6 (q 2 H), 1.1 (s, 6 H), 0.8 (t, 3 H). |
| 40 | C(O)—C₆H₅ | (R)CH₃ | H | Cl | 2 | S | | (CDCl₃): 8.2 (m, 2 H), 8.0 (m, 2 H), 7.6 (m, 1 H), 7.4 (m, 2 H), |
| | | | | | | | | 7.1 (m, 1 H), 4.6 (m, 1 H), 3.6 (m, 2 H), 3.2 (m, 1 H), 2.2-1.8 (m, 3 |
| | | | | | | | | H), 1.4 (d, 3 H) |
| 41 | —C(O)—C₆H₅ | H | Cl | H | 1 | S | | (CDCl₃): 8.2 (m, 1 H), 8.0 (m, 2 H), 7.6 (m, 1 H), 7.5 (m, 2 H), 7.2 |
| | | | | | | | | (m, 2 H), 4.3 (m 3 H), 4.1 (m, 1 H), 3.7 (m, 1 H), 2.4 (m, 1 H), |
| | | | | | | | | 2.2 (m, 1 H). |
| 42 | C(O)—C₆H₅ | H | Cl | Cl | 1 | S | 87-89 | |
| 43 | C(O)-(4-Cl—C₆H₄) | H | H | Cl | 2 | S | | 8.2 (s, 1 H), 8.1 (s, 1 H), 7.9 (m, 2 H), 7.4 (m, 2 H), 7.1 (m, 1 H), |
| | | | | | | | | 4.3 (m, 2 H), 4.1-4.0 (m, 2 H); 3.7 (m, 1 H), 2.4 (m, 1 H), |
| | | | | | | | | 2.0 (m, 2 H), 1.8 (m, 1 H). |

### Example 44

### Benzoic acid 2-[1-(6-chloropyridin-3-yloxy)-ethyl]-pyrrolidin-1-yl ester

### Step 1: 2-(1-Hydroxyethyl)-pyrrolidine-1-carboxylic acid tert-butyl ester

A solution of 13.97 g (0.07 mol) of 2-formyl-pyrrolidine-1-carboxylic acid tert-butyl ester (racemate: CAS-Nr 117625-90-8, R-enantiomer Cas-Nr 73365-02-3, S-enantiomer CAS-Nr 69610-41-9) in 100 ml tetrahydrofuran (THF) was cooled to 0° C. and the solution was treated with 55 ml of a 1.4 M solution of methylmagensiumbromide in THF and kept for 3 h at 3° C. Then, 200 ml of a saturated aqueous solution of NH₄Cl was added. The organic phase was separated and the aqueous phase was extracted twice with 200 ml of methyl-tertbutylether. The combined organic extracts were washed with brine, dried over Na₂SO₄, filtered and the solvent was evaporated. The remaining crude product (16.7 g) was used in the next step without purification.

### Step 2: N-Boc-2-Chloro-5-(1-pyrrolidin-2-yl-ethoxy)-pyridine

A solution of 0.1 g (0.0385 mol) triphenylphosphine in 100 ml tetrahydrofuran was cooled to 0-5° C. and 6.702 g (0.0385 mol) azodiethylcarboxylate in 20 ml tetrahydrofuran were added and the mixture was stirred for 30 min. Then a solution of 6.37 g (0.0296 mol) 2-(1-Hydroxyethyl)-pyrrolidine-1-carboxylic acid tert-butyl ester from the step 1 in 20 ml of tetrahydrofuran and 4.6 g (0.0355 mol) of 2-chloro-5-hydroxypyridin were added. The mixture was stirred for 12 h, evaporated to dryness and the residue was subjected to column chromatography (cyclohexane/ethylacetate 9/1) to yield 2 g of a colorless oil. HPLM-MS: 3.86 min, M+H+=327

### Step 3 2-Chloro-5-(1-pyrrolidin-2-yl-ethoxy)-pyridine (Compound II: n=2, R²=CH₃, R³=Cl, R⁴=H)

2.00 g (0.006 mol) N-Boc-2-Chloro-5-(1-pyrrolidin-2-yl-ethoxy)-pyridine were dissolved in 170 ml of 4N HCl in 1.4 dioxane and stirred for 4 h. The volatiles were removed in vacuo and the remaining crude product was dissolved in 140 ml methanol. The obtained soluten was treated with 10.00 g (0.042 mol) of ion exchange resin (Amberlyst A-26, OH-form, loading 4.2 meq/g) for 3 h. After filtration of the resin methanol was removed to yield 1.2 g of the title compound.

¹H-NMR (400 MHz, CDCl₃), δ 8.1 (m, 1H), 7.3 (m, 2H), 4.3 (m, 1H), 3.3 (m, 1H), 3.1-2.9 (m, 2H), 2.0-1.6 (m, 4H), 1.3 (d, 3H).

### Step 4: Benzoic acid 2-[1-(6-chloropyridin-3-yloxy)-ethyl]-pyrrolidin-1-yl ester racemate (Compound I: R¹=C(O)C₆H₅, n=2, R²=CH₃, R³=Cl, R⁴=H)

1.05 g (0.0046 mol) of 2-chloro-5-(1-pyrrolidin-2-yl-ethoxy)-pyridine was treated with 1.78 g (0.0055 mol) of dibenzoylperoxide and 6.3 g (0.0552 eq.) of polyvinylpyridine as described in example 1. After workup 0.78 g of the title compound were obtained.

¹H-NMr (CDCl₃): δ 8.1 (m, 1H), 8.0 (m, 2H), 7.6 (m, 1H), 7.4 (m, 2H), 7.3 (m 1H), 7.2 (m, 1H), 4.7 (m, 1H), 3.6 (m, 2H), 3.2 (m, 1H), 2.2 (m, 1H), 2.1-1.8 (m, 3H), 1.4 (d, 3H).

By analogy to the method described in steps 1 to 3 of example 44 the following compounds of the formula II were prepared:
3-Chloro-5-(1-pyrrolidin-2-ylethoxy)-pyridine (Compound II: n=2, R²=CH₃, R³=H, R⁴=Cl)

¹H-NMR (400 MHz, CDCl₃): δ: 8.2 (m, 2H), 7.2 (s, 1H), 4.3 (m, 1H), 3.2 (m, 1H), 3.0-2.8 (m, 2H), 2.1-1.6 (m, 5H), 1.3 (d, 3).
2,3-Dichloro-5-(1-pyrrolidin-2-ylethoxy)-pyridine (Compound II: n=2, R²=CH₃, R³=Cl, R⁴=Cl)

¹H-NMR (400 MHz, CDCl₃): δ 8.0 (m, 1H), 7.3 (s, 1H), 4.3 (m, 1H), 3.2 (m, 1H), 3.0-2.8 (m, 2H), 2.1-1.6 (m, 5H), 1.3 (d, 3).

### Example 45

### 3-(2S-1-Azetidin-2-yl-ethoxy)-5-chloro-pyridine (compound IIa-S with R^{2a}=CH₃, R^{3a}=H and R^{4a}=Cl)

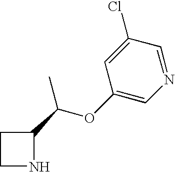

### Step 1: 2S-(1-Hydroxy-ethyl)-azetidine-1-carboxylic acid tert-butyl ester

6.900 g (0.037 mmol) of 2-formyl-azetidine-1-carboxylic acid tert-butyl ester (S-enantiomer, known from European Journal of Medicinal Chemistry (2000), 35(11), 979-988) were dissolved in 70 ml tetrahydrofuran. To the solution 30.6 ml of 1.4 M methylmagnesiumbromid in tetrahydrofuran were added at 0° C. After stirring the reaction mixture for 6 h at 0° C. the reaction was quenched with 60 ml of saturated aqueous NH₄Cl-solution. The volatiles were removed in vacuo and the residue extracted three times with 200 ml of ethyl acetate. The combined organic phases were dried over Na₂SO₄, the solution was filtered and the solvent removed in vacuo to yield 6.6 g crude product as a mixture of SR and SS diastereomers.

¹H-NMr (CDCl₃): 4.4 (br,), 4.1-3.7 (m,), 2.4-2.2 (m), 1.4 (2 s), 1.1, 1.0 (2 d).

### Step 2: 2-[1-(5-Chloro-pyridin-3-yloxy)-ethyl]-azetidine-1-carboxylic acid tert-butyl ester

According to step 2 of example 446.60 g (0.033 mol) of 2S-(1-Hydroxy-ethyl)azetidine-1-carboxylic acid tert-butyl ester and 5.099 g (0.0394 mol) 3-hydroxy-5-chloropyridine were reacted in the presence of 11.18 g (0.043 mol) Triphenylphosphine and 7.43 g (0.043 mol) azodiethylcarboxylate to yield after column chromatographie (cyclohexane/ethyl acetate 100/2.5 (v/v) 2.69 g of the title compound.

¹H NMR (CDCl₃) 8.3 (s, 1H), 8.2 (s, 1H), 7.3 (br, 1H), 5.0-4.7 (br, 1H), 4.3 (m, 1H), 3.9-3.7 (m, 2H), 2.5-2.2 (m. 2H), 1.4 (s, 9H), 1.2 (d, 3H).

### Step 3 2S-3-(1-Azetidin-2-yl-ethoxy)-5-chloro-pyridine

A solution of 2.6 g (0.0083 mol) of 2-[1-(5-Chloro-pyridin-3-yloxy)-ethyl]-azetidine-1-carboxylic acid tert-butyl ester in 10 ml of Dichloromethane was treated for 2 h with 50 ml of 4 M HCl in 1.4 Dioxane. The reaction mixture was evaporated to dryness and the residue redissolved in 50 ml Methanol. After addition of 25 g of Amberlyst A-26 (OH-form, 4.2 meq/g) the mixture was agitated for 2 h, filtered, the filter was washed with 200 ml of methanol and the combined methanol solutions were evaporated to dryness to yield 1.6 g of the title compound as a mixture of SR and SS-diasteromeres.

¹H NMR (CDCl₃) 8.2 (s, 1H), 8.1 (s, 1H), 7.6 (s, 1H), 4.5 (m, 1H), 3.8 (m, 1H), 3.5 (m, 1H), 3.2-3.0 (m, 1H), 2.2-2.0 (m, 2H), 1.3 (d, 3H),

The action of the compounds of the formula I against pests was demonstrated by the following experiments:

### II. ASSESSMENT OF THE ACTIVITY AGAINST ANIMAL PEST

The active compounds were formulated in a mixture of 50 vol.-% acetone: 50 vol.-% water. A nonionic surfactant (Kinetic®) was included in the solution at a volume of 0.01% v/v.

In the following tests, the formulated solutions of the active compounds were diluted to an active ingredient concentration of 300 ppm and the diluted solutions were applied in the below mentioned tests.

The following compounds were tested:
Compound A: R-3-Chloro-5-(pyrrolidin-2-ylmethyloxy)-pyridine (Compound II: n=2, R²=CH₃, R³=Cl, R⁴=H)
Compound B: Example 3
Compound C: Example 40
Compound D: Example 43 Compound E: Example 44
Compound F: 2-Chloro-5-(1-pyrrolidin-2-ylethoxy)-pyridine (Compound II: n=2, R²=CH₃, R³=Cl, R⁴=H)
Compound G: 3-Chloro-5-(1-pyrrolidin-2-ylethoxy)-pyridine (Compound II: n=2, R²=CH₃, R³=H, R⁴=Cl)
Compound H: 2S-3-(1-Azetidin-2-yl-ethoxy)-5-chloro-pyridine

### II.1 Cotton Aphid (Aphis Gossypil), Mixed Life Stages

Cotton plants at the cotyledon stage were infested prior to treatment by placing a heavily infested leaf from the main aphid colony on top of each cotyledon. The aphids were allowed to transfer overnight and the host leaf was removed. The infested cotyledons were then dipped and agitated in the test solution for 3 seconds and allowed to dry in a fume hood. Test plants were maintained under fluorescent lighting in a 24-hr photoperiod at 25° C. and 20-40% relative humidity. Aphid mortality on the treated plants, relative to mortality on untreated check plants, was determined after 5 days. In this test, test compounds B, C and F at 300 ppm provided 100%, and 86-99% mortality, respectively, of cotton aphid (Aphis gossypii, mixed life stages) in comparison with untreated controls. Test compound A at 300 ppm provided 75-85% mortality, respectively, of cotton aphid (Aphis gossypii, mixed life stages) in comparison with untreated controls.

II.2 Green Peach Aphid (Myzus persicae), Mixed Life Stages

Bell pepper plants at the first true-leaf stage were infested prior to treatment by placing heavily infested leaves from the main aphid colony on top of the treatment plants. The aphids were allowed to transfer overnight to accomplish an infestation of 30-40 aphids per plant and the host leaves were removed. The infested leaves of the test plants were then dipped and agitated in the test solution for 3 seconds and allowed to dry in a fume hood. Test plants were maintained under fluorescent lighting in a 24-hr photoperiod at 25° C. and 20-40% relative humidity. Aphid mortality on the treated plants, relative to mortality on untreated check plants, was determined after 5 days. Test compound A provided 85% mortality while test compounds B and C each provided 100% mortality of green peach aphid (Myzus persicae, mixed life stages) in comparison with untreated controls, when applied at 300 ppm. Test Compounds D, E, F, G and H each provided >85% mortality of green peach aphid (Myzus persicae, mixed life stages) in comparison with untreated controls, when applied at 300 ppm.

1. Pyridine compounds of the general formula I 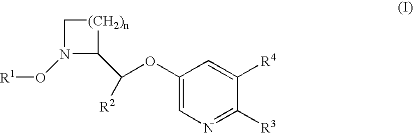
wherein n is 1 or 2,
R¹ is selected from the group consisting of hydrogen, C₁-C₆-alkyl, C₂-C₆-alkenyl, C₂-C₆-alkynyl, C₃-C₆-cycloalkyl, C₃-C₆-cycloalkyl-C₁-C₄-alkyl, phenyl and phenyl-C₁-C₄-alkyl, wherein C₁-C₆-alkyl, C₂-C₆-alkenyl, C₂-C₆-alkynyl, C₃-C₆-cycloalkyl, C₃-C₆-cycloalkyl-C₁-C₄-alkyl may be unsubstituted or may be partially or fully halogenated or carry a radical R^{a} selected from OH, CN, C₁-C₄-alkoxy, C₁-C₄-haloalkoxy, —S—C₁-C₄-alkyl, —S(O)—C₁-C₄-alkyl, —S(O)₂—C₁-C₄-alkyl, —S—C₁-C₄-haloalkyl, —S(O)—C₁-C₄-haloalkyl, —S(O)₂—C₁-C₄-haloalkyl, NH₂, NH—C₁-C₄-alkyl, N(C₁-C₄-alkyl)₂, —C(O)—C₁-C₄-alkyl, —C(O)—C₁-C₄-haloalkyl, —C(O)—O—C₁-C₄-alkyl, C(O)NH₂, —C(O)—NH—C₁-C₄-alkyl
and —C(O)—N—(C₁-C₄-alkyl)₂ and wherein phenyl and the phenyl ring in phenyl-C₁-C₄-alkyl may carry 1, 2, 3, 4 or 5 radicals selected from halogen, C₁-C₄-haloalkyl, NO₂ and radicals R^{a},
or R¹ is a radical of the formula C(O)—R⁵;
R² is hydrogen or C₁-C₄-alkyl;
R³, R⁴ are selected independently of one another from H, halogen, CN, C₁-C₄-alkyl, C₁-C₄-haloalkyl, C₁-C₄-alkoxy, —S—C₁-C₄-alkyl, —S(O)—C₁-C₄-alkyl, S(O)₂—C₁-C₄-alkyl, —S—C₁-C₄-haloalkyl, —S(O)—C₁-C₄-haloalkyl,
S(O)₂—C₁-C₄-haloalkyl; and
R⁵ is selected from hydrogen, C₁-C₆-alkyl, C₂-C₆-alkenyl, C₂-C₆-alkynyl, C₁-C₄-haloalkyl, C₂-C₄-haloalkenyl, C₂-C₄-haloalkynyl, wherein C₁-C₆-alkyl, C₂-C₆-alkenyl, C₂-C₆-alkynyl and C₁-C₆-alkoxy, may be unsubstituted or may carry a radical R^{b} which is selected from selected from OH, CN, C₁-C₄-alkoxy, C₁-C₄-haloalkoxy, —S—C₁-C₄-alkyl, —S(O)—C₁-C₄-alkyl, —S(O)₂—C₁-C₄-alkyl, —S—C₁-C₄-haloalkyl, —S(O)—C₁-C₄-haloalkyl, —S(O)₂—C₁-C₄-haloalkyl, NH₂, NH—C₁-C₄-alkyl, N(C₁-C₄-alkyl)₂, —C(O)—C₁-C₄-alkyl, —C(O)—C₁-C₄-haloalkyl, —C(O)—O—C₁-C₄-alkyl, C(O)NH₂, —C(O)—NH—C₁-C₄-alkyl and —C(O)—N—(C₁-C₄-alkyl)₂,
R⁵ may also be O—R⁶, NR⁷R⁸, C₃-C₆-cycloalkyl, aryl, hetaryl, C₃-C₆-cycloalkyl-C₁-C₄-alkyl, aryl-C₁-C₄-alkyl and hetaryl-C₁-C₄-alkyl, wherein cycloalkyl, aryl and hetaryl in the six last mentioned radicals may be unsubstituted or may carry 1, 2, 3, 4 or 5 radicals R^{c}, selected from halogen, OH, CN, NO₂, C₁-C₄-alkyl, C₁-C₄-haloalkyl, O—R⁹, S(O)ₖR¹⁰, NR¹″-C(O)—R¹², NR—SO₂—R¹⁴, —C(O)—R¹⁵, —C(O)—O—R, —NR¹⁷R¹⁸, —C(O)—NR¹⁹R²⁰ and —S(O)₂—NR²¹R²², it being possible for two radicals R^{c} which are bound to adjacent carbon atoms to form a C₃-C₆ alkylen moiety or a
O—(C₁-C₄-alkylene)-O moiety, wherein the hydrogen atoms of alkylene may be replaced by halogen atoms;
k is 0, 1 or 2;
R⁶, R⁹R¹⁰, R¹⁴ independently of each other are C₁-C₄-alkyl, C₁-C₄-haloalkyl, C₃-C₄-alkenyl, C₃-C₄-alkynyl, C₃-C₆-cycloalkyl, C₃-C₆-cycloalkyl-C₁-C₄-alkyl, phenyl and phenyl-C₁-C₄-alkyl, wherein phenyl in the last two mentioned radicals may be unsubstituted or may carry 1, 2, 3, 4 or 5 radicals R^{d}, where R^{d} is selected from halogen, OH, CN, NO₂, C₁-C₄-alkyl, C₁-C₄-haloalkyl, C₁-C₄-alkoxy and C₁-C₄-haloalkoxy;
R⁷, R⁸, R¹¹, R¹³, R¹⁷, R¹⁸, R¹⁹, R²⁰, R²¹, R²² independently of each other are selected from hydrogen and C₁-C₄-alkyl, C₃-C₄-alkenyl and C₃-C₄-alkynyl;
R¹², R¹⁵, R¹⁶ independently of each other are selected from hydrogen, C₁-C₄-alkyl, C₁-C₄-haloalkyl, C₃-C₄-alkenyl, C₃-C₄-alkynyl, C₃-C₆-cycloalkyl, C₃-C₆-cycloalkyl-C₁-C₄-alkyl, phenyl and phenyl-C₁-C₄-alkyl, wherein phenyl in the last two mentioned radicals may be unsubstituted or may carry 1, 2, 3, 4 or 5 radicals R^{e}, where R^{e} is selected from halogen, OH, CN, NO₂, C₁-C₄-alkyl, C₁-C₄-haloalkyl, C₁-C₄-alkoxy and C₁-C₄-haloalkoxy;
the pyridine-N-oxides of I and the salts thereof.

2. The compounds as claimed in claim 1, wherein R¹ in formula I is a radical of the formula C(O)—R⁵ wherein R⁵ is as defined above.

3. The compounds as claimed in claim 2, wherein R⁵ in formula I is selected from
—C₁-C₆-alkyl, C₂-C₆-alkenyl, C₂-C₆-alkynyl, C₁-C₄-haloalkyl, C₂-C₄-haloalkenyl, C₂-C₄-haloalkynyl, wherein C₁-C₆-alkyl, C₂-C₆-alkenyl, C₂-C₆-alkynyl may be unsubstituted or may carry a radical R^{b} as defined above,
C₁-C₆-alkoxy, which may be unsubstituted or may carry a radical R^{b} as defined above
—NH₂, NH—C₁-C₄-alkyl, N(C₁-C₄-alkyl)₂,
C₃-C₆-cycloalkyl, C₃-C₆-cycloalkyl-C₁-C₄-alkyl,
phenyl, naphthyl, phenyl-C₁-C₄-alkyl,
hetaryl, hetaryl-C₁-C₄-alkyl
wherein cycloalkyl, phenyl, naphthyl and hetaryl in the seven last mentioned radicals may be unsubstituted or may carry 1, 2, 3, 4 or 5 radicals R^{c} as defined above.

4. The compounds as claimed in claim 3, wherein R⁵ in formula I is selected from H, NH₂, C₁-C₄-alkoxy, C₁-C₆-alkyl, C₂-C₆-alkenyl, C₂-C₆-alkynyl, C₁-C₂-haloalkyl, C₃-C₆-cycloalkyl, naphthyl, phenyl, pyridyl, thienyl, furyl, pyrazolyl, oxazolyl, isoxazolyl, thiazolyl, 1,2,4-oxdiazolyl, 1,3,4-oxdiazolyl, wherein the last 11 radicals may be unsubstituted or may carry 1, 2 or 3 radicals selected from halogen, CN, NO₂, C₁-C₄-alkyl, C₁-C₄-haloalkyl, NH—SO₂—(C₁-C₄-alkyl), C₁-C₄-alkoxy and C₁-C₄-haloalkoxy, or R⁵ is a radical A-R^{aa} wherein
A is C₁-C₃-alkylen, and
R^{aa} is selected from C₁-C₄-alkoxy, —S—C₁-C₄-alkyl, —S(O)—C₁-C₄-alkyl, S(O)₂—C₁-C₄-alkyl, NH₂, NH—C₁-C₄-alkyl, N(C₁-C₄-alkyl)₂, C₃-C₆-cycloalkyl, phenyl, pyridyl, thienyl, furyl, pyrazolyl, oxazolyl, isoxazolyl, thiazolyl, 1,2,4-oxdiazolyl or 1,3,4-oxdiazolyl, wherein the last 11 radicals may be unsubstituted or may carry 1, 2 or 3 radicals selected from halogen, CN, NO₂, C₁-C₄-alkyl, C₁-C₄-haloalkyl, NH—SO₂—(C₁-C₄-alkyl), C₁-C₄-alkoxy and C₁-C₄-haloalkoxy.

5. The compounds as claimed in claim 1, wherein at least one radical R³ or R⁴ in formula I is different from hydrogen.

6. The compounds as claimed in claim 1, wherein the radical R³ and R⁴ in formula I are selected, independently of each other, from hydrogen and halogen.

7. The compounds as claimed in claim 1, wherein n in formula I is 1.

8. The compounds as claimed in claim 7, wherein R¹ in formula I is R⁵—C(O)— and wherein R⁵ is selected from
—C₁-C₆-alkyl, C₂-C₆-alkenyl, C₂-C₆-alkynyl, C₁-C₄-haloalkyl, C₂-C₄-haloalkenyl, C₂-C₄-haloalkynyl, wherein C₁-C₆-alkyl, C₂-C₆-alkenyl, C₂-C₆-alkynyl may be unsubstituted or may carry a radical R^{b} as defined above,
C₁-C₆-alkoxy, which may be unsubstituted or may carry a radical R^{b} as defined above
NH₂, NH—C₁-C₄-alkyl, N(C₁-C₄-alkyl)₂,
C₃-C₆-cycloalkyl, C₃-C₆-cycloalkyl-C₁-C₄-alkyl,
phenyl, naphthyl, phenyl-C₁-C₄-alkyl,
hetaryl, and hetaryl-C₁-C₄-alkyl,
wherein cycloalkyl, phenyl, naphthyl and hetaryl in the seven last mentioned radicals may be unsubstituted or may carry 1, 2, 3, 4 or 5 radicals R^{c} as defined in claim 1.

9. The compounds as claimed in claim 8, wherein R⁵ in formula I is selected from H, NH₂, C₁-C₄-alkoxy, C₁-C₆-alkyl, C₂-C₆-alkenyl, C₂-C₆-alkynyl, C₁-C₂-haloalkyl, C₃-C₆-cycloalkyl, naphthyl, phenyl, pyridyl, thienyl, furyl, pyrazolyl, oxazolyl, isoxazolyl, thiazolyl, 1,2,4-oxdiazolyl, 1,3,4-oxdiazolyl, wherein the last 11 radicals may be unsubstituted or may carry 1, 2 or 3 radicals selected from halogen, CN, NO₂, C₁-C₄-alkyl, C₁-C₄-haloalkyl, NH—SO₂—(C₁-C₄-alkyl), C₁-C₄-alkoxy and C₁-C₄-haloalkoxy, or a radical A-R^{aa} wherein
A is C₁-C₃-alkylen, and
R^{aa} is selected from C₁-C₄-alkoxy, —S—C₁-C₄-alkyl, —S(O)—C₁-C₄-alkyl, S(O)₂—C₁-C₄-alkyl, NH₂, NH—C₁-C₄-alkyl, N(C₁-C₄-alkyl)₂, C₃-C₆-cycloalkyl, phenyl, pyridyl, thienyl, furyl, pyrazolyl, oxazolyl, isoxazolyl, thiazolyl, 1,2,4-oxdiazolyl or 1,3,4-oxdiazolyl, wherein the last 11 radicals may be unsubstituted or may carry 1, 2 or 3 radicals selected from halogen, CN, NO₂, C₁-C₄-alkyl, C₁-C₄-haloalkyl, NH—SO₂—(C₁-C₄-alkyl), C₁-C₄-alkoxy and C₁-C₄-haloalkoxy.

10. The compounds as claimed in claim 7, wherein the radical R³ and R⁴ in formula I are selected, independently of each other, from hydrogen and halogen.

11. The compounds as claimed in claim 1, wherein n in formula I is 2.

12. The compounds as claimed in claim 11, wherein R¹ in formula I is R⁵—C(O)— and wherein R⁵ is selected from
C₁-C₆-alkyl, C₂-C₆-alkenyl, C₂-C₆-alkynyl, C₁-C₄-haloalkyl, C₂-C₄-haloalkenyl, C₂-C₄-haloalkynyl, wherein C₁-C₆-alkyl, C₂-C₆-alkenyl, C₂-C₆-alkynyl may be unsubstituted or may carry a radical R^{b} as defined above,
C₁-C₆-alkoxy, which may be unsubstituted or may carry a radical R^{b} as defined above
NH₂, NH—C₁-C₄-alkyl, N(C₁-C₄-alkyl)₂,
C₃-C₆-cycloalkyl, C₃-C₆-cycloalkyl-C₁-C₄-alkyl, phenyl and phenyl-C₁-C₄-alkyl, wherein cycloalkyl and phenyl in the last four mentioned radicals may be unsubstituted or may carry 1, 2, 3, 4 or 5 radicals R^{c} as defined in claim 1.

13. The compounds as claimed in claim 12, wherein R⁵ in formula I is selected from H, NH₂, C₁-C₄-alkoxy, C₁-C₆-alkyl, C₂-C₆-alkenyl, C₂-C₆-alkynyl, C₁-C₂-haloalkyl, C₃-C₆-cycloalkyl, phenyl, wherein the last 2 radicals may be unsubstituted or may carry 1, 2 or 3 radicals selected from halogen, CN, NO₂, C₁-C₄-alkyl, C₁-C₄-haloalkyl, NH—SO₂—(C₁-C₄-alkyl), C₁-C₄-alkoxy and C₁-C₄-haloalkoxy, or a radical A-R^{aa} wherein
A is C₁-C₃-alkylen, and
R^{aa} is selected from C₁-C₄-alkoxy, —S—C₁-C₄-alkyl, —S(O)—C₁-C₄-alkyl, S(O)₂—C₁-C₄-alkyl, NH₂, NH—C₁-C₄-alkyl, N(C₁-C₄-alkyl)₂, C₃-C₆-cycloalkyl and phenyl, wherein phenyl may be unsubstituted or may carry 1, 2 or 3 radicals selected from halogen, CN, NO₂, C₁-C₄-alkyl, C₁-C₄-haloalkyl, NH—SO₂—(C₁-C₄-alkyl), C₁-C₄-alkoxy and C₁-C₄-haloalkoxy.

14. The compounds as claimed in claim 11, wherein the radical R³ and R⁴ in formula I are selected, independently of each other, from hydrogen and halogen.

15. A composition for combating pests, selected from arthropods and nematodes, which comprises a pesticidally effective amount of at least one pyridine compound of the general formula I 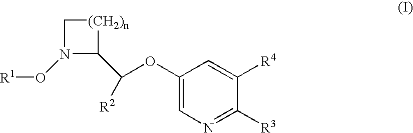
wherein n is 1 or 2,
R¹ is selected from the group consisting of hydrogen, C₁-C₆-alkyl, C₂-C₆-alkenyl, C₂-C₆-alkynyl, C₃-C₆-cycloalkyl, C₃-C₆-cycloalkyl-C₁-C₄-alkyl, phenyl and phenyl-C₁-C₄-alkyl, wherein C₁-C₆-alkyl, C₂-C₆-alkenyl, C₂-C₆-alkynyl, C₃-C₆-cycloalkyl, C₃-C₆-cycloalkyl-C₁-C₄-alkyl may be unsubstituted or may be partially or fully halogenated or carry a radical R^{a} selected from OH, CN, C₁-C₄-alkoxy, C₁-C₄-haloalkoxy, —S—C₁-C₄-alkyl, —S(O)—C₁-C₄-alkyl, —S(O)₂—C₁-C₄-alkyl, —S—C₁-C₄-haloalkyl, —S(O)—C₁-C₄-haloalkyl, —S(O)₂—C₁-C₄-haloalkyl, NH₂, NH—C₁-C₄-alkyl, N(C₁-C₄-alkyl)₂, —C(O)—C₁-C₄-alkyl, —C(O)—C₁-C₄-haloalkyl, —C(O)—O—C₁-C₄-alkyl, C(O)NH₂, —C(O)—NH—C₁-C₄-alkyl
and —C(O)—N—(C₁-C₄-alkyl)₂ and wherein phenyl and the phenyl ring in phenyl-C₁-C₄-alkyl may carry 1, 2, 3, 4 or 5 radicals selected from halogen, C₁-C₄-haloalkyl, NO₂ and radicals R^{a},
or R¹ is a radical of the formula C(O)—R⁵;
R² is hydrogen or C₁-C₄-alkyl,
R³, R⁴ are selected independently of one another from H, halogen, CN, C₁-C₄-alkyl, C₁-C₄-haloalkyl, C₁-C₄-alkoxy, —S—C₁-C₄-alkyl, —S(O)—C₁-C₄-alkyl, S(O)₂—C₁-C₄-alkyl, —S—C₁-C₄-haloalkyl, —S(O)—C₁-C₄-haloalkyl,
S(O)₂—C₁-C₄-haloalkyl; and
R⁵ is selected from hydrogen, C₁-C₆-alkyl, C₂-C₆-alkenyl, C₂-C₆-alkynyl, C₁-C₄-haloalkyl, C₂-C₄-haloalkenyl, C₂-C₄-haloalkynyl, wherein C₁-C₆-alkyl, C₂-C₆-alkenyl, C₂-C₆-alkynyl and C₁-C₆-alkoxy, may be unsubstituted or may carry a radical R^{b} which is selected from selected from OH, CN, C₁-C₄-alkoxy, C₁-C₄-haloalkoxy, —S—C₁-C₄-alkyl, —S(O)—C₁-C₄-alkyl, —S(O)₂—C₁-C₄-alkyl, —S—C₁-C₄-haloalkyl, —S(O)—C₁-C₄-haloalkyl, —S(O)—C₁-C₄-haloalkyl, NH₂, NH—C₁-C₄-alkyl, N(C₁-C₄-alkyl)₂, —C(O)—C₁-C₄-alkyl, —C(O)—C₁-C₄-haloalkyl, —C(O)—O—C₁-C₄-alkyl, C(O)NH₂, —C(O)—NH—C₁-C₄-alkyl and —C(O)—N—(C₁-C₄-alkyl)₂,
R⁵ may also be O—R⁶, NR⁷R⁸, C₃-C₆-cycloalkyl, aryl, hetaryl, C₃-C₆-cycloalkyl-C₁-C₄-alkyl, aryl-C₁-C₄-alkyl and hetaryl-C₁-C₄-alkyl, wherein cycloalkyl, aryl and hetaryl in the six last mentioned radicals may be unsubstituted or may carry 1, 2, 3, 4 or 5 radicals R^{c}, selected from halogen, OH, CN, NO₂, C₁-C₄-alkyl, C₁-C₄-haloalkyl, O—R⁹, S(O)ₖR¹⁰, NR¹¹—C(O)—R¹², NR¹³—SO₂—R¹⁴, —C(O)—R¹⁵, —C(O)—O—R¹⁶, —NR¹⁷R¹⁸, —C(O)—NR¹⁹R²⁰ and —S(O)₂—NR²¹R²², it being possible for two radicals R^{c} which are bound to adjacent carbon atoms to form a C₃-C₆ alkylen moiety or a
O—(C₁-C₄-alkylene)-O moiety, wherein the hydrogen atoms of alkylene may be replaced by halogen atoms;
k is 0, 1 or 2,
R⁶, R⁹R¹⁰, R¹⁴ independently of each other are C₁-C₄-alkyl, C₁-C₄-haloalkyl, C₃-C₄-alkenyl, C₃-C₄-alkynyl, C₃-C₆-cycloalkyl, C₃-C₆-cycloalkyl-C₁-C₄-alkyl, phenyl and phenyl-C₁-C₄-alkyl, wherein phenyl in the last two mentioned radicals may be unsubstituted or may carry 1, 2, 3, 4 or 5 radicals R^{d}, where R^{d} is selected from halogen, OH, CN, NO₂, C₁-C₄-alkyl, C₁-C₄-haloalkyl, C₁-C₄-alkoxy and C₁-C₄-haloalkoxy,
R⁷, R⁸, R¹¹, R¹³, R¹⁷, R¹⁸, R¹⁹, R²⁰, R²¹, R²² independently of each other are selected from hydrogen and C₁-C₄-alkyl, C₃-C₄-alkenyl and C₃-C₄-alkynyl,
R¹², R¹⁵, R¹⁶ independently of each other are selected from hydrogen, C₁-C₄-alkyl, C₁-C₄-haloalkyl, C₁-C₄-alkenyl, C₃-C₄-alkynyl, C₃-C₆-cycloalkyl, C₃-C₆-cycloalkyl-C₁-C₄-alkyl, phenyl and phenyl-C₁-C₄-alkyl, wherein phenyl in the last two mentioned radicals may be unsubstituted or may carry 1, 2, 3, 4 or 5 radicals R^{e}, where R^{e} is selected from halogen, OH, CN, NO₂, C₁-C₄-alkyl, C₁-C₄-haloalkyl, C₁-C₄-alkoxy and C₁-C₄-haloalkoxy;
the pyridine-N-oxides of I and the salts thereof,
or a salt thereof and at least one inert carrier and/or at least one surfactant.

16. A method for combating pests, selected from arthropods and nematodes, which comprises contacting said pests, their habitat, breeding ground, food supply, plant, seed, soil, area, material or environment in which the animal pests are growing or may grow, or the materials, plants, seeds, soils, surfaces or spaces to be protected from an attack of or infestation by said pest, with a pesticidally effective amount of a pyridine compound of the general formulae I or II or a pyridine-N-oxide thereof 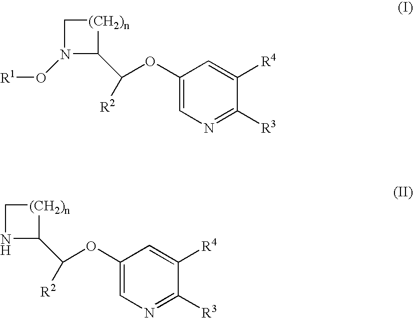
wherein n is 1 or 2,
R¹ is selected from the group consisting of hydrogen, C₁-C₆-alkyl C₂-C₆-alkenyl, C₂-C₆-alkynyl, C₃-C₆-cycloalkyl C₃-C₆-cycloalkyl-C₁-C₄-alkyl, phenyl and phenyl-C₁-C₄-alkyl, wherein C₁-C₆-alkyl, C₂-C₆-alkenyl, C₂-C₆-alkynyl, C₃-C₆-cycloalkyl, C₃-C₆-cycloalkyl-C₁-C₄-alkyl may be unsubstituted or may be partially or fully halogenated or carry a radical R^{a} selected from OH, CN, C₁-C₄-alkoxy, C₁-C₄-haloalkoxy, —S—C₁-C₄-alkyl, —S(O)—C₁-C₄-alkyl, —S(O)₂—C₁-C₄-alkyl, —S—C₁-C₄-haloalkyl, —S(O)—C₁-C₄-haloalkyl, —S(O)₂—C₁-C₄-haloalkyl, NH₂, NH—C₁-C₄-alkyl, N(C₁-C₄-alkyl)₂, —C(O)—C₁-C₄-alkyl, —C(O)—C₁-C₄-haloalkyl, —C(O)—O—C₁-C₄-alkyl, C(O)NH₂, —C(O)—NH—C₁-C₄-alkyl
and —C(O)—N—(C₁-C₄-alkyl)₂ and wherein phenyl and the phenyl ring in phenyl-C₁-C₄-alkyl may carry 1, 2, 3, 4 or 5 radicals selected from halogen, C₁-C₄-haloalkyl, NO₂ and radicals R^{a},
or R¹ is a radical of the formula C(O)—R⁵;
R² is hydrogen or C₁-C₄-alkyl,
R³, R⁴ are selected independently of one another from H, halogen, CN, C₁-C₄-alkyl, C₁-C₄-haloalkyl, C₁-C₄-alkoxy, —S—C₁-C₄-alkyl, —S(O)—C₁-C₄-alkyl, S(O)₂—C₁-C₄-alkyl, —S—C₁-C₄-haloalkyl, —S(O)—C₁-C₄-haloalkyl,
S(O)₂—C₁-C₄-haloalkyl; and
R⁵ is selected from hydrogen, C₁-C₆-alkyl, C₂-C₆-alkenyl, C₂-C₆-alkynyl, C₁-C₄-haloalkyl, C₂-C₄-haloalkenyl, C₂-C₄-haloalkynyl, wherein C₁-C₆-alkyl, C₂-C₆-alkenyl, C₂-C₆-alkynyl and C₁-C₆-alkoxy, may be unsubstituted or may carry a radical R^{b} which is selected from selected from OH, CN, C₁-C₄-alkoxy, C₁-C₄-haloalkoxy, —S—C₁-C₄-alkyl, —S(O)—C₁-C₄-alkyl, —S(O)₂—C₁-C₄-alkyl, —S—C₁-C₄-haloalkyl, —S(O)—C₁-C₄-haloalkyl, —S(O)₂—C₁-C₄-haloalkyl, NH₂, NH—C₁-C₄-alkyl, N(C₁-C₄-alkyl)₂, —C(O)—C₁-C₄-alkyl, —C(O)—C₁-C₄-haloalkyl, —C(O)—O—C₁-C₄-alkyl, C(O)NH₂, —C(O)—NH—C₁-C₄-alkyl and —C(O)—N—(C₁-C₄-alkyl)₂,
R⁵ may also be O—R⁶, NR⁷R⁸, C₃-C₆-cycloalkyl, aryl, hetaryl, C₃-C₆-cycloalkyl-C₁-C₄-alkyl, aryl-C₁-C₄-alkyl and hetaryl-C₁-C₄-alkyl, wherein cycloalkyl, aryl and hetaryl in the six last mentioned radicals may be unsubstituted or may carry 1, 2, 3, 4 or 5 radicals R^{c}, selected from halogen, OH, CN, NO₂, C₁-C₄-alkyl, C₁-C₄-haloalkyl, O—R⁹, S(O)ₖR¹⁰, NR¹¹—C(O)—R¹², NR¹³—SO₂—R¹⁴, —C(O)—R¹⁵, —C(O)—O—R¹⁶, —NR¹⁷R¹⁸, —C(O)—NR¹⁹R²⁰ and —S(O)₂—NR²¹R²², it being possible for two radicals R^{c} which are bound to adjacent carbon atoms to form a C₃-C₆ alkylen moiety or a
O—(C₁-C₄-alkylene)-O moiety, wherein the hydrogen atoms of alkylene may be replaced by halogen atoms;
k is 0, 1 or 2,
R⁶, R⁹R¹⁰, R¹⁴ independently of each other are C₁-C₄-alkyl, C₁-C₄-haloalkyl, C₃-C₄-alkenyl, C₃-C₄-alkynyl, C₃-C₆-cycloalkyl, C₃-C₆-cycloalkyl-C₁-C₄-alkyl, phenyl and phenyl-C₁-C₄-alkyl, wherein phenyl in the last two mentioned radicals may be unsubstituted or may carry 1, 2, 3, 4 or 5 radicals R^{d}, where R^{d} is selected from halogen, OH, CN, NO₂, C₁-C₄-alkyl, C₁-C₄-haloalkyl, C₁-C₄-alkoxy and C₁-C₄-haloalkoxy;
R⁷, R⁸, R¹¹, R¹³, R¹⁷, R¹⁸, R¹⁹, R²⁰, R²¹, R²² independently of each other are selected from hydrogen and C₁-C₄-alkyl, C₃-C₄-alkenyl and C₃-C₄-alkynyl;
R¹², R¹⁵, R¹⁶ independently of each other are selected from hydrogen, C₁-C₄-alkyl C₁-C₄-haloalkyl, C₃-C₄-alkynyl, C₃-C₄-cycloalkyl, C₃-C₆-cycloalkyl-C₁-C₄-alkyl, phenyl and phenyl-C₁-C₄-alkyl, wherein phenyl in the last two mentioned radicals may be unsubstituted or may carry 1, 2, 3, 4 or 5 radicals R^{e}, where R^{e} is selected from halogen, OH, CN, NO₂, C₁-C₄-alkyl, C₁-C₄-haloalkyl, C₁-C₄-alkoxy and C₁-C₄-haloalkoxy;
the pyridine-N-oxides and the salts thereof.

17. The method as claimed in claim 16, wherein the pests are insects.

18. A method for protecting crops from attack or infestation by pests, selected from arthropods and nematodes, the method comprising contacting a crop with a pesticidally effective amount of at least one pyridine compound of the general formulae I. 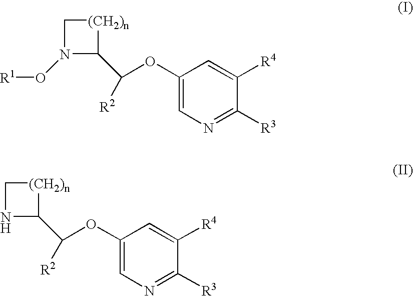
wherein n is 1 or 2,
R¹ is selected from the group consisting of hydrogen, C₁-C₆-alkyl, C₂-C₆-alkenyl, C₂-C₆-alkynyl, C₃-C₆-cycloalkyl, C₃-C₆-cycloalkyl-C₁-C₄-alkyl, phenyl and phenyl-C₁-C₄-alkyl, wherein C₁-C₆-alkyl, C₂-C₆-alkenyl, C₂-C₆-alkynyl, C₃-C₆-cycloalkyl, C₃-C₆-cycloalkyl-C₁-C₄-alkyl may be unsubstituted or may be partially or fully halogenated or carry a radical R^{a} selected from OH, CN, C₁-C₄-alkoxy, C₁-C₄-haloalkoxy, —S—C₁-C₄-alkyl, —S(O)—C₁-C₄-alkyl, —S(O)₂—C₁-C₄-alkyl, —S—C₁-C₄-haloalkyl, —S(O)—C₁-C₄-haloalkyl, —S(O)₂—C₁-C₄-haloalkyl, NH₂, NH—C₁-C₄-alkyl, N(C₁-C₄-alkyl)₂, —C(O)—C₁-C₄-alkyl, —C(O)—C₁-C₄-haloalkyl, —C(O)—O—C₁-C₄-alkyl, C(O)NH₂, —C(O)—NH—C₁-C₄-alkyl
and —C(O)—N—(C₁-C₄-alkyl)₂ and wherein phenyl and the phenyl ring in phenyl-C₁-C₄-alkyl may carry 1, 2, 3, 4 or 5 radicals selected from halogen, C₁-C₄-haloalkyl, NO₂ and radicals R^{a},
or R¹ is a radical of the formula C(O)—R⁵;
R² is hydrogen or C₁₋₄-alkyl
R³, R⁴ are selected independently of one another from H, halogen, CN, C₁-C₄-alkyl, C₁-C₄-haloalkyl, C₁-C₄-alkoxy, —S—C₁-C₄-alkyl, —S(O)—C₁-C₄-alkyl, S(O)₂—C₁-C₄-alkyl, —S—C₁-C₄-haloalkyl, —S(O)—C₁-C₄-haloalkyl, S(O)₂—C₁-C₄-haloalkyl; and
R⁵ is selected from hydrogen, C₁-C₆-alkyl, C₂-C₆-alkenyl, C₂-C₆-alkynyl, C₁-C₄-haloalkyl, C₂-C₄-haloalkenyl, C₂-C₄-haloalkynyl, wherein C₁-C₆-alkyl, C₂-C₆-alkenyl, C₂-C₆-alkynyl and C₁-C₆-alkoxy, may be unsubstituted or may carry a radical R^{b} which is selected from selected from OH, CN, C₁-C₄-alkoxy, C₁-C₄-haloalkoxy, —S—C₁-C₄-alkyl, —S(O)—C₁-C₄-alkyl, —S(O)₂—C₁-C₄-alkyl, —S—C₁-C₄-haloalkyl, —S(O)—C₁-C₄-haloalkyl, —S(O)₂—C₁-C₄-haloalkyl, NH₂, NH—C₁-C₄-alkyl, N(C₁-C₄-alkyl)₂, —C(O)—C₁-C₄-alkyl, —C(O)—C₁-C₄-haloalkyl, —C(O)—O—C₁-C₄-alkyl, C(O)NH₂—C(O)—NH—C₁-C₄-alkyl and —C(O)—N—(C₁-C₄-alkyl)₂,
R⁵ may also be O—R⁶, NR⁷R⁸, C₃-C₆-cycloalkyl, aryl, hetaryl, C₃-C₆-cycloalkyl-C₁-C₄-alkyl, aryl-C₁-C₄-alkyl and hetaryl-C₁-C₄-alkyl, wherein cycloalkyl, aryl and hetaryl in the six last mentioned radicals may be unsubstituted or may carry 1, 2, 3, 4 or 5 radicals R^{c}, selected from halogen, OH, CN, NO₂, C₁-C₄-alkyl, C₁-C₄-haloalkyl, O—R⁹, S(O)ₖR¹⁰, NR¹¹—C(O—R¹², NR¹³—SO₂—R¹⁴, —C(O)—R¹⁵, —C(O)—O—R¹⁶, —NR¹⁷R¹⁸, —C(O)—NR¹⁹R²⁰ and —S(O)—NR²¹R²², it being possible for two radicals R^{c} which are bound to adjacent carbon atoms to form a C₃-C₆ alkylen moiety or a
O—(C₁-C₄-alkylene)-O moiety, wherein the hydrogen atoms of alkylene may be replaced by halogen atoms;
k is 0, 1 or 2;
R⁶, R⁹R¹⁰, R¹⁴ independently of each other are C₁-C₄-alkyl, C₁-C₄-haloalkyl, C₃-C₄-alkenyl, C₃-C₄-alkynyl, C₃-C₆-cycloalkyl, C₃-C₆-cycloalkyl-C₁-C₄-alkyl, phenyl and phenyl-C₁-C₄-alkyl, wherein phenyl in the last two mentioned radicals may be unsubstituted or may carry 1, 2, 3, 4 or 5 radicals R^{d}, where R^{d} is selected from halogen, OH, CN, NO₂, C₁-C₄-alkyl, C₁-C₄-haloalkyl, C₁-C₄-alkoxy and C₁-C₄ haloalkoxy;
R⁷, R⁸, R¹¹, R¹³, R¹⁷, R¹⁸, R¹⁹, R²⁰, R²¹, R²² independently of each other are selected from hydrogen and C₁-C₄-alkyl, C₃-C₄-alkenyl and C₃-C₄-alkynyl;
R¹², R¹⁵, R¹⁶ independently of each other are selected from hydrogen, C₁-C₄-alkyl, C₁-C₄-haloalkyl, C₃-C₄-alkenyl, C₃-C₄-alkynyl, C₃-C₆-cycloalkyl, C₃-C₆-cycloalkyl-C₁-C₄-alkyl, phenyl and phenyl-C₁-C₄-alkyl, wherein phenyl in the last two mentioned radicals may be unsubstituted or may carry 1, 2, 3, 4 or 5 radicals R^{e}, where R^{e} is selected from halogen, OH, CN, NO₂, C₁-C₄-alkyl, C₁-C₄-haloalkyl, C₁-C₄-alkoxy and C₁-C₄-haloalkoxy,
the pyridine-N-oxides and the salts thereof.

19. A method for protecting seeds from infestation by pests, selected from arthropods and nematodes, the method comprising contacting a crop with a pesticidally effective amount of at least one pyridine compound of the general formulae I or II 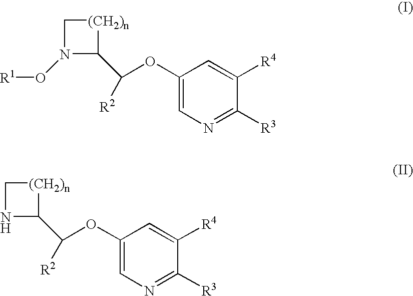
wherein n is 1 or 2,
R¹ is selected from the group consisting of hydrogen, C₁-C₆-alkyl, C₂-C₆-alkenyl, C₂-C₆-alkynyl, C₃-C₆-cycloalkyl, C₃-C₆-cycloalkyl-C₁-C₄-alkyl, phenyl and phenyl-C₁-C₄-alkyl, wherein C₁-C₆-alkyl, C₂-C₆-alkenyl, C₃-C₆-alkynyl, C₃-C₆-cycloalkyl, C₃-C₆-cycloalkyl-C₁-C₄-alkyl may be unsubstituted or may be partially or fully halogenated or carry a radical R^{a} selected from OH, CN, C₁-C₄-alkoxy, C₁-C₄-haloalkoxy, —S—C₁-C₄-alkyl, —S(O)—C₁-C₄-alkyl, —S(O)₂—C₁-C₄-alkyl, —S—C₁-C₄-haloalkyl, —S(O)—C₁-C₄-haloalkyl, —S(O)₂—C₁-C₄-haloalkyl, NH₂, NH—C₁-C₄-alkyl, N(C₁-C₄-alkyl)₂, —C(C₁-C₄-alkyl, —C(O)—C₁-C₄-haloalkyl, —C(O)—O—C₁-C₄-alkyl, C(O)NH₂, —C(O)—NH—C₁-C₄-alkyl
and —C(O)—N—(C₁-C₄-alkyl) and wherein phenyl and the phenyl ring in phenyl-C₁-C₄-alkyl may carry 1, 2, 3, 4 or 5 radicals selected from halogen, C₁-C₄-haloalkyl, NO₂ and radicals R^{a},
or R¹ is a radical of the formula C(O)—R⁵;
R² is hydrogen or C₁-C₄-alkyl,
R³, R⁴ are selected independently of one another from H, halogen, CN, C₁-C₄-alkyl, C₁-C₄-haloalkyl, C₁-C₄-alkoxy, —S—C₁-C₄-alkyl, —S(O)—C₁-C₄-alkyl, S(O)₂—C₁-C₄-alkyl, —S—C₁-C₄-haloalkyl, —S(O)—C₁-C₄-haloalkyl,
S(O)₂—C₁-C₄-haloalkyl; and
R⁵ is selected from hydrogen, C₁-C₆-alkyl, C₂-C₆-alkenyl, C₂-C₆-alkynyl, C₁-C₄-haloalkyl, C₂-C₄-haloalkenyl, C₂-C₄-haloalkynyl, wherein C₁-C₆-alkyl, C₂-C₆-alkenyl, C₂-C₆-alkynyl and C₁-C₆-alkoxy, may be unsubstituted or may carry a radical R^{b} which is selected from selected from OH, CN, C₁-C₄-alkoxy, C₁-C₄-haloalkoxy, —S—C₁-C₄-alkyl, —S(O)—C₁-C₄-alkyl, —S(O)₂—C₁-C₄-alkyl, —S—C₁-C₄-haloalkyl, —S(O)—C₁-C₄-haloalkyl, —S(O)₂—C₁-C₄-haloalkyl, NH₂, NH—C₁-C₄-alkyl, N(C₁-C₄-alkyl)₂, —C(C₁-C₄-alkyl, —C(O)—C₁-C₄-haloalkyl, —C(O)—O—C₁-C₄-alkyl, C(O)NH₂, —C(O)—NH—C₁-C₄-alkyl and —C(O)—N—(C₁-C₄-alkyl)₂,
R⁵ may also be O—R, NR⁷R⁸, C₃-C₆-cycloalkyl, aryl, hetaryl, C₃-C₆-cycloalkyl-C₁-C₄-alkyl, aryl-C₁-C₄-alkyl and hetaryl-C₁-C₄-alkyl, wherein cycloalkyl, aryl and hetaryl in the six last mentioned radicals may be unsubstituted or may carry 1, 2, 3, 4 or 5 radicals R^{c}, selected from halogen, OH, CN, NO₂, C₁-C₄-alkyl, C₁-C₄-haloalkyl, O—R⁹, S(O)ₖR¹⁰, NR¹¹—C(O)—R¹², NR¹³—SO₂—R¹⁴, —C(O)—R¹⁵, —C(O)—O—R¹⁶, —NR¹⁷R¹⁸, —C(O)—NR¹⁹R²⁰ and —S(O)—NR²¹R²², it being possible for two radicals R^{c} which are bound to adjacent carbon atoms to form a C₃-C₆alkylen moiety or a
O—(C₁-C₄-alkylene)-O moiety, wherein the hydrogen atoms of alkylene may be replaced by halogen atoms;
k is 0, 1 or 2;
R⁶, R⁹R¹⁰, R¹⁴ independently of each other are C₁-C₄-alkyl, C₁-C₄-haloalkyl, C₃-C₄-alkenyl, C₃-C₄-alkynyl, C₃-C₆-cycloalkyl, C₃-C₆-cycloalkyl-C₁-C₄-alkyl, phenyl and phenyl-C₁-C₄-alkyl, wherein phenyl in the last two mentioned radicals may be unsubstituted or may carry 1, 2, 3, 4 or 5 radicals R^{d}, where R^{d} is selected from halogen, OH, CN, NO₂, C₁-C₄-alkyl, C₁-C₄-haloalkyl, C₁-C₄-alkoxy and C₁-C₄-haloalkoxy;
R⁷, R⁸, R¹¹, R¹³, R¹⁷, R¹⁸, R¹⁹, R²⁰, R²¹, R₂₂ independently of each other are selected from hydrogen and C₁-C₄-alkyl, C₃-C₄-alkenyl and C₃-C₄-alkynyl,
R¹², R¹⁵, R¹⁶ independently of each other are selected from hydrogen, C₁-C₄-alkyl, C₁-C₄-haloalkyl, C₃-C₄-alkenyl, C₃-C₄-alkynyl, C₃-C₆-cycloalkyl, C₃-C₆-cycloalkyl-C₁-C₄-alkyl, phenyl and phenyl-C₁-C₄-alkyl, wherein phenyl in the last two mentioned radicals may be unsubstituted or may carry 1, 2, 3, 4 or 5 radicals R^{e}, where R^{e} is selected from halogen, OH, CN, NO₂, C₁-C₄-alkyl, C₁-C₄-haloalkyl, C₁-C₄-alkoxy and C₁-C₄-haloalkoxy,
the pyridine-N-oxides and the salts thereof.

20. A method for protecting non-living materials from attack or infestation by pests, selected from arthropods and nematodes, the method comprising contacting the non-living material with a pesticidally effective amount of at least one pyridine compound of formulae I or II 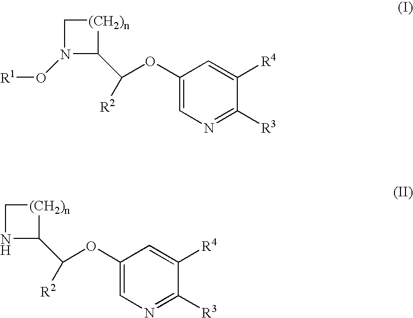
wherein n is 1 or 2,
R¹ is selected from the group consisting of hydrogen, C₁-C₆-alkyl, C₂-C₆-alkenyl, C₂-C₆-alkynyl, C₁-C₆-cycloalkyl, C₃-C₆-cycloalkyl-C₁-C₄-alkyl, phenyl and phenyl-C₁-C₄-alkyl, wherein C₁-C₆-alkyl, C₂-C₆-alkenyl, C₂-C₆-alkynyl, C₃-C₆-cycloalkyl, C₃-C₆-cycloalkyl-C₁-C₄-alkyl may be unsubstituted or may be partially or fully halogenated or carry a radical R^{a} selected from OH, CN, C₁-C₄-alkoxy, C₁-C₄-haloalkoxy, —S—C₁-C₄-alkyl, —S(O)—C₁-C₄-alkyl, —S(O)₂—C₁-C₄-alkyl, —S—C₁-C₄-haloalkyl, —S(O)—C₁-C₄-haloalkyl, —S(O)₂—C₁-C₄-haloalkyl, NH₂, NH—C₁-C₄-alkyl, N(C₁-C₄-alkyl)₂, —C(O)—C₁-C₄-alkyl, —C(O)—C₁-C₄-haloalkyl, —C(O)—O—C₁-C₄-alkyl, C(O)NH₂, —C(O)—NH—C₁-C₄-alkyl
and —C(O)—N—(C₁-C₄-alkyl)₂ and wherein phenyl and the phenyl ring in phenyl-C₁-C₄-alkyl may carry 1, 2, 3, 4 or 5 radicals selected from halogen, C₁-C₄-haloalkyl, NO₂ and radicals R^{a},
or R¹ is a radical of the formula C(O)—R⁵;
R² is hydrogen or C₁₋₄-alkyl;
R³, R⁴ are selected independently of one another from H, halogen, CN, C₁-C₄-alkyl, C₁-C₄-haloalkyl, C₁-C₄-alkoxy, —S—C₁-C₄-alkyl, —S(O)—C₁-C₄-alkyl, S(O)₂—C₁-C₄-alkyl, —S—C₁-C₄-haloalkyl, —S(O)—C₁-C₄-haloalkyl, S(O)₂—C₁-C₄-haloalkyl; and
R⁵ is selected from hydrogen, C₁-C₆-alkyl, C₂-C₆-alkenyl, C₂-C₆-alkynyl, C₁-C₄-haloalkyl, C₂-C₄-haloalkenyl, C₂-C₄-haloalkynyl, wherein C₁-C₆-alkyl, C₂-C₆-alkenyl, C₂-C₆-alkynyl and C₁-C₆-alkoxy, may be unsubstituted or may carry a radical R^{b} which is selected from selected from OH, CN, C₁-C₄-alkoxy, C₁-C₄-haloalkoxy, —S—C₁-C₄-alkyl, —S(O)—C₁-C₄-alkyl, —S(O)₂—C₁-C₄-alkyl, —S—C₁-C₄-haloalkyl, —S(O)—C₁-C₄-haloalkyl, —S(O)₂—C₁-C₄-haloalkyl, NH₂, NH—C₁-C₄-alkyl, N(C₁-C₄-alkyl)₂, —C(O)—C₁-C₄-alkyl, —C(O)—C₁-C₄-haloalkyl, —C(O)—O—C₁-C₄-alkyl, C(O)NH₂, —C(O)—NH—C₁-C₄-alkyl and —C(O)—N—(C₁-C₄-alkyl)₂,
R⁵ may also be O—R⁶, NR⁷R⁸, C₃-C₆-cycloalkyl, aryl, hetaryl, C₃-C₆-cycloalkyl-C₁-C₄-alkyl, aryl-C₁-C₄-alkyl and hetaryl-C₁-C₄-alkyl, wherein cycloalkyl, aryl and hetaryl in the six last mentioned radicals may be unsubstituted or may carry 1, 2, 3, 4 or 5 radicals R^{c}, selected from halogen, OH, CN, NO₂, C₁-C₄-alkyl, C₁-C₄-haloalkyl, O—R⁹, S(O)ₖR¹⁰, NR¹¹—C(O)—R¹², NR¹³—SO₂—R¹⁴, —C(O)—R¹⁵, —C(O)—O—R¹⁶, —NR¹⁷R¹⁸, —C(O)—NR¹⁹R²⁰ and —S(O)₂—NR²¹R²², it being possible for two radicals R^{c} which are bound to adjacent carbon atoms to form a C₃-C₆ alkylen moiety or a
O—(C₁-C₄-alkylene)-O moiety, wherein the hydrogen atoms of alkylene may be replaced by halogen atoms;
k is 0, 1 or 2;
R⁶, R⁹R¹⁰, R¹⁴ independently of each other are C₁-C₄-alkyl, C₁-C₄-haloalkyl, C₃-C₄-alkenyl, C₃-C₄-alkynyl, C₃-C₆-cycloalkyl, C₃-C₆-cycloalkyl-C₁-C₄-alkyl, phenyl and phenyl-C₁-C₄-alkyl, wherein phenyl in the last two mentioned radicals may be unsubstituted or may carry 1, 2, 3, 4 or 5 radicals R^{d}, where R^{d} is selected from halogen, OH, CN, NO₂, C₁-C₄-alkyl C₁-C₄-haloalkyl, C₁-C₄-alkoxy and C₁-C₄-haloalkoxy;
R⁷, R⁸, R¹¹R¹³, R¹⁷, R¹⁸, R¹⁹, R²⁰, R²¹, R²² independently of each other are selected from hydrogen and C₁-C₄-alkyl, C₁-C₄-alkenyl and C₁-C₄-alkynyl;
R¹², R¹⁵, R¹⁶ independently of each other are selected from hydrogen, C₁-C₄-alkyl, C₁-C₄-haloalkyl, C₃-C₄-alkenyl, C₃-C₄-alkynyl, C₃-C₆-cycloalkyl, C₃-C₆-cycloalkyl-C₁-C₄-alkyl, phenyl and phenyl-C₁-C₄-alkyl, wherein phenyl in the last two mentioned radicals may be unsubstituted or may carry 1, 2, 3, 4 or 5 radicals R^{e}, where R^{e} is selected from halogen, OH, CN, NO₂, C₁-C₄-alkyl, C₁-C₄-haloalkyl, C₁-C₄-alkoxy and C₁-C₄-haloalkoxy;
the pyridine-N-oxides and the salts thereof.

21. A compound of the general formula Ia 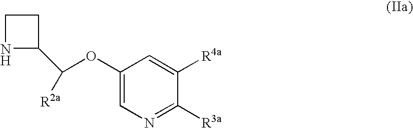
wherein
R^{2a} is C₁-C₄-alkyl;
R^{3a}, R^{4a} are selected independently of one another from H, halogen, CN, C₁-C₄-alkyl, C₁-C₄-alkoxy, —S—C₁-C₄-alkyl, —S(O)—C₁-C₄-alkyl, S(O)₂—C₁-C₄-alkyl, —S—C₁-C₄-haloalkyl, —S(O)—C₁-C₄-haloalkyl, S(O)₂—C₁-C₄-haloalkyl; and
the pyridine N-oxide and the salts thereof.

22. The compounds as claimed in claim 21, wherein the radical R^{3a} and R^{4a} in formula IIa are selected, independently of each other, from hydrogen and halogen.
